SEQUENCE LISTING (52 sequences)

SEQ ID NO: 1 - DNA, 3000 bp, Zea mays
  source 1..3000: mol_type = genomic DNA
cttccagcatagtatgaaactagttttctctccaattcaatactagtacatgcgagctggtacgcgctactcgtacgccgctactacgatacactagctaggcagctagctagttgcagctagccttgcacgggccgggaggtacctgtatatgtcactaactcctccatgccagtatataataacgatgtgtgcatgtgcatgtgacacaggcaccaccacacacacgtccacaccatgagctctgattgctccgtttcaccggccggccggccgggtgcgccgctcatgcgcgcgcggcaccgtcaccctgtcgtccctgcagctgcaggccagccagcccggaacggcgcgcgtcccggcctcgatcgcaagcaaaccgcccaccctgacctcgctcgacagctaggtgtagcccccgattcgccgatcgtccatcgagaccgctgtgcgagtacatgtgcttgccagacgtacgtacgtgtacgtacgcacgcagcaaagccgccgtacgcgtgccgggcggtacggtcgatcgagctagcgcagcagcccccggccggccggccccacggaggagggtgaagggcatgcaatgcatgcacggagacggcgagacccaacgggcccagcgacggcgccagacgcaaaggcgaagagatggggtaaagactgatggtacgcgtgctgttgacatctgcgcgcgtgtgtgcccagccagagccaggacacgacacagggctcacatgggatgggaagggcccattcatccggccgtccgtgaatgtgatgtgccttgctttgttctcgtttcatcaccagcagtcaccactcaaacaataatacacgtttgtcagtcatgcatcaatactactcgctccaagcaactaattaacattcatatgacaataatacatagtttaaaaattacaaatattccaaaacggaggtagtatgaaaaaaatagccctcctctgatatgcatgcctagttactagttgggctaaagtaacccaatttggttatggttacctagctatatagttagttagttagctatcaagtagttccaacacttgcatacaaaaaatagccctcctctagtttaaatgccccaaagctaatttaggttagttttgagctacctagcaattagtacgatccaaacaggctctaagcctattattgatatatatacacataattaagatgggctattggcagtgcaggggatctaggcttgattggcaggtggtgaatgaggtctaggagagtattaagcaatgcaagtgtctctatatatgcatgggggcagctcgcgtacttggtggtagagagggcaccgaccgtcagaagaagaacctaagggtcaaccacagacgacgagagtttatatcgtcattaggttttacaatgtcgtttgcagcttcagcgcgccttggaaaactcggcaagcgataatttcgttgcaacttgataaaaaaatgctatggaagttcatcggatgccttattagcttgagaaaactatttgctatactatactatttctgtttaaattcttagcttacaaatttaattaatgcactaccggaaacgagtgctttgccgagtgtccgaagtactcggcaaagccttaaaaacactcgacaaaggttttaccgagtgtcacactcggcaaagaaggctcggcaaacagtgcatcgtcaaagccttttttgccgagtactttttctcgggcactcggcaaagctttctcgggcactcggcaaacaatacatgtttgccgagtgtttgacactcggcaaacatgtctttgccgattgtggtcatgtgctgagtgtcctgcgctcggtaaatcagctcgttatcgagagccgtactttgccgagtgcggctatcggcaaagccttctttgccgagtgcccgacaaaaggcactcgacaaagagtccgacactcggcaaagcctcggatttcgatagtaatggtgaaattataaatagaatgttataggattcacttaaagaacaaaaaaagtatcttcattatttctttatgtagtaatccgtatacttgctttgcctataaattagagtgtattctcgcttcctgataagttcaaatatttttttgacattgaccaaacatatacaaaacattattaatatttctgataaataataagtatcgttgcattaatagttacttatttgaatatgtaaacattaccaatatttttttgaacttcaccaaacaagttacagccaccaaataagttgctttgtaccaaaaaagttgcagccgccagacttaacactgcacaccatcaaaatcaacgtttcagaccgttagataaccaatatcctttactcattcaaattcaatagacaatactatttggattatctctctccctcacgtcctcggcgcccctctccctcactcgcaccaccgttgtcgccgtcggcaccaccgtcaaccccttccctcgcaccgccaccggtattgtcgacctcttccctcatctctacgggatcatagcgttagcacaaactatatgctagtagatttaatgatctagtgtagtcaaattaaaaaaaattagtgagttactttggtaaccaatagcctccttttctagtatctgtacttccctagagcctagacaaggtgcaagaggaaaccaataaacgactcccaaatttttccaaaaggtttgcacagctcccaagtaaaagctcagttttagtacccgtgcaagttccacgtaggaatttaacagcaaatttttttttgattctttaacaacaaataaataataaaattagtccatctagaccctgtacagttcccacacgtttcaaacggacgaaattaataattacctaaaaatgtgcaggtcgtagcaaagtataattaaattaatacgaaagaataggcacggaaatgccggtaccaaagatataaaaaagggtagaggggaatggaagagtccagga

SEQ ID NO: 2 - DNA, 3000 bp, Zea mays
  source 1..3000: mol_type = genomic DNA
ggacaaaactctacttacatgtttacttagtttaaatctgatgtggcagcattgaaactcatcaccggtaaatatttgtcacattttcttttggtggtgcttcacgcatacgccctacccttgaccattttttgtacaatataatgatatgtattcttatagttctcagagtttatgagatatttgaacacaaattaaaattaccttacatgtcgtttatagatttttagaagtttacaagacaactgcatgttttgcaatttaagggtctgaccaaattttggtctaaacaacatgttcttttacaagagaaagcagaagaaccgcatgtttttcttctcatagagtaataaatgaatagaaaagagttgtgtgcgtctacacaaacggagctagatatcacataaatattccaagtggatagaaaatacaaagaactagttatcacaagaggccataatttatatttatatttactattaataaagtgctctccttaaattgagttagtatacttgtataatttatttcacaaataaatccttttctaattaagccatgtgcaaaactaagacaaactatgttgaaaaggcaaaacaattggtcatcgtattatcacgtagccgtttttagttctagcgcctctcgatttctttttactcgtgtatgtggtcgatgctctacgctgcatagttttcttttctcattctaccctctccacaaagctcattctcattctcacttcgaacacgcgacccctcaagtaaatatgctcgtagctatcactacacctcatgtgtgtttatgtctacatctattatagtaaaaacacatactacatctctctcaaagcccacctgctacccttgcacaatgtgtagtagtagataaatatcaccgagattattaaattatatttttggattgagggagtagtatttaaagaagagccttgcatgcaatttcttttgtttgaataatgttttcaacttaaattccttttttgcatgtgcgacatttattctccgtaatatttttccatggatttaacttataagtcattttcataaaccaatgccaaagatgaatccatgtttcttacttggaaaccccaaacaaacaccactagcacgaggcgctcgcgttggcgtcgctcatcgacgacgagaagaaacttggcgactgccagttcgacctatagaagcagtcctacgtgccccatgcgccgctgtgtacggtaagctccggcgcagccgccgcttggacgcggtccagagcggttgcaccgcgctgtccattgtcaaacatggggacctcatggtcgtcgccaatgccgacgactctcgggttgttctgggcaccgcaaccgacaaagacgccatcacgccgtccagctcatcatccacctgaagcccaacctgccacgtaagttgctactgatcggccatgaattcttgtgcactgggatgacggccattgttgctattgtgtgagcgttctcgaacaagatgtatgtagaggagtagcatatccggtggtgcaacgaccaggtgtactacctcgctgatgagcccggagtgcacttcatttggcagcccagccaacagtcatcggtactcaccatgtcgcgtgcgttcgacgactactatatcaaggattgtgacgtcatctcggcgccggaggtgagcagaggaggaccgacaacaatgaccactcaccatcgtcggggtagcttttgtgttggcctgtctttaattctcttcgcaaacacacacttgtattttttttgtttaagcaagcgtgtatggcggtgcgtgcctgatgactaacgatgtacgacggaacttctatcggcaggtgtggtacgtgctctctaatgatgagaccatgcaaattatgacatatatcgaagtctgtatgatactgatggttgcaatgtagtagtgaaacaactaaattaaaataacaaaatttatgtatggctaggatcacaaatggattatgaaatattttcttataataatataagacacattttgtatataagttatcatagtattatatgttcccgttgcaacgcacgggtactatacatgaggagtgggttgattccactaaaattgatggaatgaacttattatgcatcaactcataaagcatagagtgattccacaaaccaaacacaccattagtttattgaccaacctcagttctttgatatataaaagttgtttgatccgtgtgcaacaaatagtttttcaggaatttgggacgtaccccatctcttaaatgtctagactgattggacatattaatctaattttagtagctaagtattagctctcaaacacctcctgggaatgctgccctaaaaatggaatatcggtacgtcggatcggagaaaggttggggtatcgtggtccaaagcatgcgaaagcaacggcgcagggctggaaacctgagaccaggtgaggtgcacgacgacatgcatatggtttgggttaggcctaggagctggttctctctccatgcatggtcagctcgccgcttttgccgccttcgtgtatggcttgccccatgccatgcatcgcgccgtgtacacttgtggcgtaggggcggggccgccgcatcggagcgcccccgtttcggcacggtcctcccagttttagggtaaacccagctagggtagtgggggtaactggccagcgccactccaaatctaccctccttcaatttaaagctgagaaatactgtagtatatactagagtagagcgagagaagggagatgtggatagatgggtggatggggacgcgtgaaaaagatgcgagagagaagagacgaccggacaggcagccacacacagtaacagtagtgaaccctgccccttttcccggtctctccactgatattccgctcctgtccctgtcctccccggacggagctaatagagccggctctgctcatcattatatcgtcgcgcacaaatgcgaagcctagc

SEQ ID NO: 3 - DNA, 3000 bp, Zea mays
  source 1..3000: mol_type = genomic DNA
ttcttcccgtttccacctaacaggcaaaacacttaggcccctcctcatctaaggatttagggcgggtgcattctgccacccggccggagatttcctccccgacactgtcaatctttattctagtaaggttctaccctcatatcactacatcacaagtctagatgacaagagaggttctcacctgtgtagggaacgaagcgcggattaggagagagcttggttccggtgctggctaaatggcggcgcggggcgtggagtcacatggctcgagtccgacgacgatgatggtggcgcggggcgacgatggaggtggcgtggcacggcgaccgaggcgacggggcgctgacagaggcagcgaccgaggcggcagcgctctgtggcgtgcggtgagtgagagagtgaaaggagaaggagagggcccgcgctggtctattaatgtaacgttctttgccaagtgttcgcgatctcgcactcaacaaagttgtttttattttaatttttttgcggagtgtcctggatctgacactcggcaatgaattattagccgagcgtccactgacaagcactcagtaaaaaatgttttatttttttaaaaaaaaaactttgccgatcgtcccccgaccgacactcgacaaagaatactttgacgtgtgtcttcctttgacactcggcaaagtatgtttttattcttttctttttgcaccaaactttttgtggtgtgttcctacactatgtagatctacatgttcaaatttggcacagttataaacttttttgctatacctattagattttgttcgtttaattgaattccttcgaataatttagatttgaactgcaagtcactcgaaaatagaaaactatgaatacaaacactagtacagataagctctatagtggcggttgtaaacctatttacagtgacgtttttcgtaaccgctagtgctaggggccagtagaaattatcatttttataggcgggtaactgagaaccgaaaatcgattttcactgacggtcggtgtaataaaaccgccaatgaaaatcgtttccaggaaacataaaacggattttaaaaatagcaagaaaaatatttttattagggaggcctcgcggcccacccacccccacctgtctgtcttcgtcgaagtcgaaaatcgattttcaccgacggtcggtgtaataaaaccgccaatgaaaatcgtttccaagaaacataaaacggattttaaaaatagcaagaaaaatatttttattaggcaggcctcgcggcccacccacccccacctgtctatcttcgtcgaagtcgcaagtcgcggaattttttgtgcactacgcggttgttaggaatcgaaccactgacctcaccctcatgcttaccctcctctatcactccggctatgacatgttttgtgtctagtttttagttttgttgcccacatattacaaccaactgagtgtaaattgcttgtttgagaccgtaaacgaattcaaataaagcagttgtcaactacaaagttgaataatttttgaagttctaaaactttcattttgacattttttcatccgaggtcgtttgcaaaatttgaattttaaatttaacaaatttagatacaatttttagagccgaaatgatttcaaataaaaaagttgtcaactacaaaattttataacttttagaaatctacaacttttattttgatggttttgtcatacgatgtcgtttgaaaaactcgaaaaattaaggataaaaatgatttctagtggcggttccttaagaaaaccgccactagaaatcgatttctacaggcgggttcttaaaaaaatcgccactagaaatcatggatttctacagacggttttcataaggaaccgcctctagaaatacgatttctagtggcggttttcttaagaaaccgccactaaagatagcataggcggttgataaccgaatccgcctataaaaatataccgcaccgcaggttttgagcctttttctactagtgaaaaatgctattcatgttatttagcacaagttacggcctattttaggaacatatcggaattgtcgagcaccatgctcacgaaacatgaccgtgaacttgtcatccaactgttttaaaattgtataaaatataagcaaagtcagaaaatcatgaaatttatccacatgccatggtatcatatgtagagcctatggtaaaaaatttgaggctgtttcgagaaagttgtcacgcactatgtgcagaaacctaagtgccctacacatgaaatcttagaacttcaatatagaaacttaagtgtcctacacatgaaaccattgaagatctgatgagatttcctttcctgttgacagtttccatctcccggacgacggtgtttgaaaaaatagcgccaactggcacgcagagtggctatctagacatacaattgctacattagtaactacatttagtaagtttatttagagaactattggagaacaatttttttgtttacttcttaaattcaaaatttagtgagttgtttagagaactattgaagttgatcttagagatgataaggagctctaaactttatactatataatttaaagatcagattagaatccaactctatttatatttagttttaaactaaattaatttaaagctcaaacaaattataaataaatatttggatcatgattcattgccatccttagttagagttaatgcctaacaaaaaccataattcagagttcaatcacgtgggtaatcgtttttcatacgctcgtgttcaagtttaaatctttaaatatatgataaatttatcatattcttttattgtcttgtcaatctcgtcattctcaaagtcgtcgacacgacacgacacgaggccatggaagtacacaacgccattataccgcaccaagtacagtacctcttcccctgctttcggctttgccatcaaccccaaccggacagcgaggtgaagccaagttgccccgcggacgcaaagca

SEQ ID NO: 4 - DNA, 3000 bp, Zea mays
  source 1..3000: mol_type = genomic DNA
tgcccaccactggttttccagttctgggatctggtgtgggggggacgactggcagtgggatctggttttccagttctgggttccccggcgacgagtacgtaccgagattatataaatactgctttaatttggcgggcgcgcgcgcctgggagggagggacgatagggcttacgttccggcaggcactgcctgtggttcttgttcgtgttcgtgtggcgtgccccggccggatttgcatggggttggtagcgctgggctagctactatagaccgggccgccatgcatgggcgcacacggcacacagacgggaagtacttgacgccttgcctcgggcgcctcaccaccagttcagatcgatatgacagtccacacatgtcgtccaaagatcgatattcatcagtatcactactactgtatctgtatcctggcgacgcggcatatattactacataccccggccgtatatacattaggccaggcgccacactaccactacggtagagaaaaaccattgccgcccggccgggcatgggcatgcatcttctggctacgcgccgtggatgagggccctccatgctcctacctagcctagcctcctcctctcctcctggccggctacaacgggcgtcagtgcgggggagtttatggccggaccgccgggagcccggccggatccccggcggccggcgcggcctctccagctctcctcgctcgctcgcgcgtctgcaggcctgcagtttgcagcagctagctcgtcgcgaggaaaatccgcgtacggccgccgcgtcgcccgatgcgcgctcgcccagctagctagctgcccctgacttcaggcataacccacgtgcatgccatgatgccgtcgtctagctcgatcgcgctggccatatagacctacgtcgtacgtactcgcatgcgacatgtcaaaggatgcgtgacttttgctggtccagtccgcggtagaggatgggagatcaccgaaggcgacgacgagaacagctagacagagaaggaattgaaaatgtgaagcgaaattcatgaaaaaaatgaattttggttccatatgaccccagtaacgggaaagcttgcgcgccagtccacagccgacacgtatggtagcctatggtggtgtcttcgcctatctaggaggtacggaatgctggccaggtcctcttgtcctctctctctcactcatgctgtgctgtttgacatgtctcgatctctcctctcctctcctctcctcccgttggtgtgactgtagtagatcctttgcccgtgtcagaacaagctgctcctcggaccgggtaatgttaaacatcggaggagcctttgcctaggatccgtaacggggaggaaagagaaaaaaaactaaggatgattatggataccgtgtaataactgctaactacagttagcccatctcagcggactctctgccctatattgtatgtcactttctattataaactacactatacaacctatgatgtaaaataatgttttgcacgttcatatataaatcagtcgaagaaagggtgcctcactacagggaatggtttctattggacaccttagcattcaatcagtcatgtccccccccccccaaaaaaaaaatgcacccatccagtcgatttttgtcatatttgaattcggtggtgctccatgcacgcgtacctgctttgaccaatttatacgatcaatatataacttacgttcttacggttcttagactttatgagactttgcaagtatgtttggatacaaatcacactaatgtgcatctttgtaaactaaattcttttgattaaatttgtaattttaaggtttaacctgtttttgttgtgtagacgacgttaggcaccgatcgtcgcttcgctatatatctttgttgtagacgacgttagactcctagattaaataagcgaaaaccgatcgtcgcttcgctatctttgtttatttgtttgtggctgctctacgctgaagagcccacaggccacagccccacacgacacgttaggcacccccacccaccatccgcgcataatataagctactgcaaaatatatgccggcggagcccgagcgagctttgtacttgctccgccgtggcctggctccaggatgctttggatttcgtgcggcgccgtacgtccaggcaaacagacaagtggagctgcatgtcctaaaagcccggcaatcaaacacgctctagcagcagcatggatcacagatatcagtcatggggtggcgctggcgcgggtgggtggccaggtggaggtgggtgcatgtcgtcgtcgtcgtcccatacagaaattggctcacgtatgtatacgctgcgtacaggcagtagtacacaattactagcaccaatgcaatccaacggatggatcttcgcacacccgccacccggttaaattaagctactcctacctctcccagtctcccttggcctgcctctatatttttgggcagcctccaccagccgggcggatggggttggatcgtcgtatctgaggcggcgtggtcgtccaaggcgaaagcaacggcgcagggctgggaccctagtaggtgcatgaggtcgtgcatggcgcgcgagatgcatggtttgggttaggcctaggaggttctctctccatggcatgggtagctcgcgccgcttggctgccgttctcgtgtatgcgcatgcaccaggcatttgcaccgcgccgtgtatatttctggcgtgggggccggcgccgcattggagctgcagccccgtttcggcacggacacgggacacctcccgttagggtaagcccggggcagtgggtaactgcccagcgccactactccgaatttaccctccttttatttttaaagcttgggagaggggagaatggatggatggatggatgtagacgcgtgaaaaagatgcgcgagaccggcagcgtgtgctacaggggcagccaggcacacacacacacagtgaccctgccccttttcccggccgtctcgctctcc

SEQ ID NO: 5 - DNA, 3000 bp, Zea mays
  source 1..3000: mol_type = genomic DNA
agatcgatcctcttcacattcttgtaatgaaggggtagcccaaggtacttcccgggaagactagagtatttgcccggaaacacctccttaagtgccggccatgatgattccaggtgccgaattgggaaaatttctgttttgtcatagttgatctttagtcctgaacatccctcgaacgcattgagaatctctttcagcacctccaagtctgatttgtcagctttaatgaatactcccgcatcatccgcgtagagggagcagcgaagcttagcccctctaggtagtacctgtcccagcagtcccacgtcagcagctttctcaatcatccgatgtagcgggtccattgctaaaatgaaaagaaagggggatagcgggtttccttgtcgcaccccacgcctgtgtttgatcgcgtttgaccgctgtccattgataattattcttgaggatgaagatctcagaatatccgctacccaattgcgccatttcagggagaacccacaggctcttaacacatctaggagatatgcccaatttagggtgtcgaaggctttagagatgtctagcttgacgaagagagctggattacctttcttatgcaaattcataatcaccctttgcacgtatagaaaattgtcgtgaattgaacggttctggatgaaagcgttctgagcactggagataatttcgttcattcttggagctagcctatttgccatgatttttgttataattttcatgaagctattgatcaggctgatcggcctaaatctatcaattatgtccgtgttttccccctttggtatgaggatgatgtttgcttcattgacccgaagcaggcttttggatctgtgatggaaaaaatcatttattgccatggagaggtcatttttgatcaactcgaagcaacttttgtagaacagcccaatgaacccgtccggcccgggggccttttcagattgaagccccatcacaacattcttgatttcactgtcatcaatcaagttgtccaactcagataactcgaaaggggtgtaccccagatggtcccaacggacagccctgctcctcctacctgtcgtgcctagcacctggctaaagtgatgttggacctcattcattttgtcttcctgactcgttagcagtttgccatcactcattaatgatctgattaaatttttccgtcttctattattggctgtcatgtgaaagaacttagtgttagcatcgcccaaccgcatccaagtgagcctggagtgttgtcttgctcgtgccgtctgcgcgaaggccatatcaaaaatcttggttttaagagaccacctgaaatccatttcttcaaaggatagttgtctaatctcttgcgctttttccaggagagtgaggatgatctgaatgatggccatctgcattttgaagttgccaacagtgttgcgcctccaggccaaaagagccttggctgtgcgagacatccttacatgaagtcgaaggatggcatcatcagagttcacagtagagttccatgcttgattgactacctcattgaacccctcaatctgaggccaaaaagattcgaatctgaacccatggtatggggaacccaaagagtggctttgcattataagaggccagtggtccgacgtcatggtgcacatggagcgaaggtcagccgaggggaataggtcgtgccactctgttgtcgctaagaatctgtcaatcttagtcatggttgggttaatttgttcatttgaccaggtgaatgttctatcggtgagctctagctcaaggaggtgcaaataatcaatcgtgttattaaattgtctcatgactcttctgttgattgatcctttgtttttctcccttgctctcctgatcatgttgaagtcacctagtaatacccattctggtttgaccacggcttggatgttacggagttcagaaaggaaaatcattttttcattgtccagctggggcccgtaaacccccgtgaggtcccaaacctcctcatgtgatctagagctaatcctgactgatagagaatatgctgctgaacatggcaagagagccatgtcaaagaaatccgaatcagcagcaataaggatgccgcccgaggcgcccagagaaggcaagtgaaaagcttgattagcaagcttggcgcctacagtttcctttaataaaacattggaccaattcgagatctttgtctcttgtaggcaaacaatggaagcaccggtggacagaattgtatgtctaacacttgctctcttcgcgtggtcatttagcccccgaacattccaagaaaaaatagcacatttttgattcataaggtaaagactgccactccacttaacacagcacgctgccaccacacatggattagcaggagagcctgctgtaaaatcctaacaggagggagaacctccaaacaagggttcgccgagcaaaaacacagcccgaccacaaccgacaacctgaaagaacaacagagatacacaggcatgctgggggacctagaccagcgcccagaagtaataacgccagcggagatacaaccgctccgagagagcctgaccatctgagaacacattggtcaccaaaagcaccaccaaccggcctagacaaagcagctcagttgacccccgcctcgacatcttcgatggccggcatcacctttctccccttctttttattcttcgctgtcttcaccttgtcttgatttaacagctccatgattgcatccatttgcttcttggagagaggctttgtgagaaggcttgtcatctgctcaaatgactcatcaaagttagtacattttgaagaactaattattattatatagaatgcactgcacatatattactattaccagttttcttgggcacagcagaaaacatgcacacgcagatagaaaaaggagaggccataaaccaaaaggctttaagaatatatgtaaagatatgtctaaatatatggctatatctggttaagcaagataacagggctctg

SEQ ID NO: 6 - DNA, 3000 bp, Zea mays
  source 1..3000: mol_type = genomic DNA
atatgtcttgttcatatttttagtttcgtaacgtatattcttcggattattgacgtagagtatggttgattcatgaattaccccattttctattttgagttaggcgcattacatacagttgaaatgattatgcttttgttcgtactactagaaccagggaattcaacatgaaaacttgatgtctgtttgcttagctttaatccgtaccaatttttactatttctatagtgctttcagttgcagcagtccaaaattcttactttatttatctttgaaaaatctgtgcttctttttaataacggctggacatgtaggtagtctaccggttgttattttatagacactgaatattgaaatagtgtcaatcttaagaattcatcgacctgaacatttaataaggtcatattcacaagaacaatataaataaatggttatatatgtaataataataataataataataattattattattattattagtggtagaggatttaaaagtttaatgctttgtacactacctaaaccaatatgttttcccctttgagggcttgttcggttatttccaatccatatggattggaggggattgatacggattggagggtattttgacttactagggattgaaaccccttcagggcttgttcggttattttcaatccatatggattggagtggattgggagggattttgacttacaagggattaaaaccccctcaatccccctcaatccatatggattggggtagaaccgaacaagccctcgatccccctcaatccatatggattggggtaaaaccgaacaagccctgaagccgcgacagacccacagttaccatgtgtgcaaagtgtatagacactgcgctgatgtagctagcgtgtgtgacactgcgtgagcccataatcaataatcatacaggcagagaacatctgtagggctaaccctaattaagaatacaatctacttaatccccctcgtagctagggtatatatacaaataattaacgggtatggcagtgcatgcatgcatgccgatgacttgcactggtaccagttcgtcactaatgattgccaattatactgcggcccttttaattatgagatggaagcagcaggaggcaagctagttcaattgtccggtttgttaagccgcattgctcaagagcacccctgcctgcgccatcgatcgccacacggtgtgtgccagtctacatcgccggcctctttatcattgtgctagtagctgcttattaggcaaagctcaattacgcagctaagccagttgatatgcataaaagggaatgattactcttaaaccggacgcactggcgctttctgcttttttttaaaggaatgctatgaaaattggtacgtacgtagtactactactatgcatatcagtttttttcttcttcttttttttcttatacatatatcagtataccagtctgtatatatgtacacacacttcgttcttcagctacgacgtacaatactgctacaccccatgggctgcgggaactgttcgtgaaaagtgacgagatgaggcagcactgtggtagatattcttgcgagtagtggcaatgtcaccctcacacttttccttgtactgccattaatcccaatttgtccttatttatctgtttaccctctctttctctctcctcggccgggcccttttttttctttgcaacatacaccaccacaatattcttcaacactctcacctcctctcattcaatactgtctctcagcctttttttttcatagtgtgtgcggccgatctatgcacctcacctttcccttgctgcagttggaggaagtgtgctgcaggtggaagaagagaggggagaagctggttcccctctccatatctctcctccccagctccctccccctctcatagatcgatcacacagcctgcaggcacagacaagagttaggctgttctacctgatcatatgaccctccagcaattctctcatccacacacaaactgaagcaagaattattgcttatctatatcctgtctgtcctttcctctatttcccccctcccctttctatttccattgtgcagcttgttattagtagatagcagtggatcgatgtgtgatggaggaaacaagctagctagctagctaccagctgaagcagcagggagaagaaggatagagagagagagagagagagaaaacatcttcttcatccatagtctcggcctttcttcaccaggtcgtctgcagatctgtctcctgtgaccttccagaggtgaggatgagcaggtctctctagctcactgcgctacatgcccatgtatgagaagttctattttagaagacaaaaaggctttgatctgcagtcttcgttctcgtcttctgggttctggccttctcttgcttggcagtggcactattattatattatctatggttcttgagatttgagcaaataaagatcgcctttttttcctttaaaaaatctgtgcttcttgtgccgtatcatcatacatgcacacccacgcatttgtacagtggatcatgcatcctccatttgtgttcatgatcatgctgttgatttgcactgctccctttatcaataattttccgcagatgagaaatctacagtctcattattgtcccctctttctttgtcccaaggtaaaaagggaagaacagaacttccccatttttcccaaagattgcacatgcatagctggctggatcagagcacatcacactccgaaaccctaatcttctccctctcctcgtgcctttcccagaacaatatataatcctccgtccaagaaaaccctagccctaagattagtatcgaaagatcgcgatgcatattattcacagctcagccaccacaccaccatatgtagagatagatatacgtatatttctttgggaaaaaacggataataatccaactaattaatcaacgtttttctactgatttattcgcaggccaagctaggccaggaaggaaaa

SEQ ID NO: 7 - DNA, 3000 bp, Zea mays
  source 1..3000: mol_type = genomic DNA
tgctgcattggattgaagaagccgggtggaaatatcttctcaagcttacacagtaacacgggtgccaatctttccaagtctgcaaccacggtccgagataactcttttgcacaaagctgatggaagaaatagctcaactctgaaagcgctagccagacatgctcagggacatagcctcgaaccatcgcaggaagaatccgttcaatccatatgtggaagtcatgactcttcatccctaagactcgcatagtagataagttcacccccctactcaggttagttgcatacccatcaggcaacatcaacatcttgatccactgaagtacttccttcctctgggccctgcttaggacgaaatcggccttaggccttctccacgtctttccgccacttggcggcttcatctctagttttggtctatcacataacgctgccagatccactcttgacttcacattatcctttgacttatcaggaatgtccataattattgcccatagtgcctcggcaacattcttttcagtgtgcatcacgtcaatgttgtgtggaaggagcaggtcgtcataataggggagccgagtcaagccagacttatgtgtccacatatgctgctcaccatatcccacaaaaccaccttctgtattggccacgagcccatctatctgttgtcgaatttcggcaccagtcatcgttgcaggtgggcggtctgtcactacgacacctttcgtaaagttcttgatgtctaggcggaatggatggtcagcaggaagaaattgtcgatgtttatcgaaggacgaatatttgccaccctttttcaaccaaatgaacctgacagcttccttgcaaactgggcatgggaacttaccgtgaacacaccaggcacagaatagcccatgcgccgataagtcatgcatggagtactggtaccaaacatgcattctgaagtttgccttcgtagctcggtcatacttccatcccccttcctcccaggcacgtaccaattcatcgatcaaaggctccatatacacgcctttgccgagtgtaggaaacatgtaggtctacatagtgtaggaacataccacaaaaagtttgggagacaaaatcaaaaaaataaaaatatactttgccgagtgtctagagaagactctagtcctttgccgagtgcccactacttggcactcggcaaagaagactctttgccgagtgccaaccctcggctctcgacaaagactgacggccgtcagctttgggacggccgctgacgaccctttgccgagcgccccctttgccgagtgtttgacactcggcaaacatgtctttgccgagtggggtcctgtgccgagtgtccagcactcggtaaagaggctcgttgccgagagcctaactttaccgagtgcggctctcggcaaagccttctttgccgagtgccggggcactcggcaaagaggccgacactcggcaaagcctcggattctggtagtgagagcatgatttaagggaaaaattaaaaccagtttcatatgtccgagataaaacattttttatcccttgcatctgatcggccacatgtagactaaaattgccatcatcgaaatcgctgatgttgaaaaacaaatcctaattcctataaaacatcagtttctctagtttccacaatcctccttttctaaaaaaattcattatatttcttctaatctatcccatctttatctttatagtttcattgtcgggcgtcaaatccgaaacaggtagatctattttttcatcacatttggcctaacaaccagatacgcttatctcttgtatgtgtaaccaagcaacaaccacgagcacttcatctccaactttccatctattttcttgtctacgccctctcttacccagattcttctacatcgccatagaggatgtagaggacctcgagcaggccatctagctggcctccgactcgccagtcgtcgcggcggcggcgtagatatatttttctcccccacgtctcctctaacaaccagataagtcttgtatattgtatgtcgtgactctccaccgccatacaatacgtggctgaacagccaagggagagaaagaggaggcacctatgacgttctcctcctttattttgcggtcctttattaatcccaacttttctatttcttttcttgttttcccctttcccacctagattcatccttgcagtgtagatctatttttcttccacacgtctagcctaacaactagataagataaaacttatatcttatactctctgttccaaattaaaatttgttttagtgaattattggattcaaacaattcttgatattttgtatatgtgtctagatttatcatcatttatttgaatatatagataaaaaacaatagttaaaacgaatattattttaagacggagcgagtatatcatcatacgatacgtggctgatctcacaatctcaacgtggtcaaagttgtgtgtgccgggccatctgcgcgtcgtgtgacaccggtgcatgcgcagccttttgttttgccgccccgcccgctccatgcatggcatgggtgcaggttctgtagctatgcccggaagcacctagctagctcgcagcctacatctgcaaactcacaaagtttgggtatcggaggcatcagcaggtcgggttcaatggaacgacggatcacgtctgtgtgtcgctttcgcagcagcggggagagcgcggggcccggcccaggacgcatggaccgatggacgcatgcagaccatttttgtttttgtttttgtttttgtttttttcctgtctaaaatgtaggtgtgctctatcttgcctcttcatgcgataatgtgtgtgtatatatatacatgcccttcactcttcttatagctcgctagcccagctttagtttatagcactctctcactcagtagtcagctccctccatttgtccattctccaaaggtagttagctaggttaggcacacgcgcgccactcgactagctagcagct

SEQ ID NO: 8 - DNA, 3000 bp, Zea mays
  source 1..3000: mol_type = genomic DNA
catggatttatatatatacattccgatagtcggctctattccagtagtgaatgcgatatatatacatacaggaatacgttggtttcttgtgatctgcagtttaaacgtaaacctcaagctgcatatataggcacattaccggaatccgaggctttgccgagtgtcgatctctttgccgaatgccttttgtcgggcactcggcaaagaaggctttgccgagagccgcactcggtaaagttaggctctcggcaacgagcctctttaccgagtgctggacactcggcaccggaccccactcggcaaaggcacgtttgccgaatgtaaaacactcggcaaaggtggcgctcggcaaagggccgtcagcggccgtcccaaagctgacggacgtcagtctttgccgagagccgagggttggcactcggcaaagagtcttctttgccgagtgccaaatagtgggcactcggcaaaggactctttgccgagtgtcttcgctagacactcggcaaagtatatttttatttttttgattttgtctcccaaactttttatggtatgttcctacactatatagacctacatgtatcatttgtggacaattataacagagttttcaatcgttagtagatttagttcgtttatttgaatttcttcggaaaattcagatttgaactgcaggtcactcgaaacttggaaaaccgtgcatgcaaaaatgatattcatgttacttagcataagttatgaccgattccagaagcggaccggaaacttcgagcaacatgctcactaaacatggtcgtgatcttgccatccacatgtttaaaaattgtataaaacacaaacaaagtcagaaaatcatgaaacttgtccacgtgtcatgatatcatatgtacaggttgtgataaaaaattgaaaaagtttcgagaaaactatgacgcgctatgtgtacaaacctaagagatccacattgaaactctatgatttcatgtatagttctcttaggtttctacacatagtgtctcacaactttatccaaacattctaaaatttttatcacagcctgtacatatgttatcatgacacgtggacaagtttcatgattttctgactttgtttgtgttttatacaatttttaaacatgtggatgacaagttcacgaccatgtttagtgagcatgttgctcgaagtttccggtacgctcatggaatcggttgtaacttatgctaagtaacatgaatatcatttgttgcatgcacggtttttcaagcttcgagtgacctacagttcaaatctgaattttccgaagaaattcaaataaacgaattaaatctactaacgattgaaaactctgttataattgtccacaaatgatacatgtaggtctacatagtgtaggaacataccacaaaaagtttgcaggccaaaacaaaaaatacaaatgtactttgccgagtgtcagctggttgacactcggcaaagtaggctttgccgagtgacccctgggtgacactcggcaaataagtgtaagttagtctttgccgagtgtcacctcgtcgacactcggcaaaggagcctttgccgagtgtccccgatctggcactcggcaaagcttattttaaaaataaaaaaaatctttgccgagtgccagatcacgggcactcggcaaagcgcgtttacatagcgctagtaattccttctttctctctcactctctcacagccgtgccgccgccgcgaccccgtcgccgccgtagacacgctgccgcgaccccgccgcgcccacgccgcgcgcgctcacgctcgcccgcacccaggccctcgccgcgcggccgcagccgcgaccgcgccgtacgccgtacgtcgtgcccccgccgtgtccccgccgtgcttgccctcgccgtgcccccgtcgcgtccccaccgtgtgtgccctcgtcgtgccctcgccgtgcccccgccgtctcgtccgcgacccgtcggcaaagaacctgacatggggaccctccctggcgggctatttgtcgagtgtcccaggggcactcggcaaagattgaatctttgccgagtgctactgggcagacactcggcaaagttaactcctttgccgagtgtcatccttgacactcggcaaagaagccatctccgtcaccccgttcccgtaacggtcacttttctttgccgagtgctcactggcacttggcaaacctctttgccgagtgcccgagaaaaagtactcggcaaagaaagatttaccgatgcactgtttgccgagccttctttgtcgagtgcgacactcggcaaaacctttgccgaatgtttttatggctttgccgagtgcttcggacactcggcaaagcgctcgtttccagcagtggcaacatgcatgacataactactgcatacatacatgcatgcaaaaccggccagcagacatgcacgatatgcataccattgcgatcgagtcggagcctccacgcgtcggctaaccgtccggcaggcggcgggccggggacaccacgaggagcacctctctcccatctctaggccacacagcaacacaatattatcttcgccgtcgcccgcaattgtttagctgtactttcaatagactttgctaagcacgctagaccaaactaatcgtttgagatcttctgtgcgcgcagcactacagcatgcatgtggccaccgtttgtctctcttgtgtgttcttattcttatatagtgaaaacaatactactatacaagacataaaattaggtgtagtttatttatttactggtactacttctttagtcataattaggtgtactagcagcagctacgccctgacggagacaagcacacacaatagaaaagagcgccctcgtgctcagagtgctcgcctttagtttcgtggctactatatactagctgcgacgatttttattttctttctcttctgggctatcattagcacggccatttaggggtaatgagcagcggccaataaacaaagccctgagatg

SEQ ID NO: 9 - DNA, 3000 bp, Zea mays
  source 1..3000: mol_type = genomic DNA
cctggcggccagccggccaccgcggcaccgcatggcaccgcggcaccgcctaccagtgacccgccacagctcacagcggcgcagccggtcagccgtccagccgacgtcctcttccaccaaggctgccagcgtcctcttccaccagggcagcccgcagcggcaccaaccagtagcctgcactctgcactctgcagccggctcgccactcgccagttcagccagtgcaatcggccactcgccagttcgggttttttcggttaaaccgaaaaccgaaccgaaaaattcggttaactgaaggttcggtttttgattttctgcagaaggtttcggttaccgttttctagaaactgaatttataaaaaaaaccaaaaaaccgaaccgaaaaaaacggttaaccgattgcccaggcctgcataaagccgagacttctttcttttctatttgtatttttcctattttaaattcaagtttcaaatagattacaaattatgaataaatttgagttttgaaattcaaatacaagttgaatgcagagatcaaaacccaacatggtatgcaactttattacctaatgtttattttattcctttgaataatacaaatgcttaattcataagagagagggaaaagaattcacccatatcagcaattgaaaatctttatgtttattaattgatttacaatttaccttctagttttgagacctcaatctaacttggaataatcaaaatacaatattaccatttacttatttttaagaaacaatttcaaagctaatagattccttattaaaggcatttataatctgaatcctttgaatagcttttctacaagattttagtgtgttacaggtaaccctcgctttttgctaagtaacttaatgttacttcaccttgcatcattaataaccttcttgcatatcataacatacatgtatctcacacattgcattcgttaggttgcaacctcgctgacagagaatacgtgttgattccagagcaaggagttgagcaagaggaagtacaggagctagctcccgagcctaccacgaaagatctccccacagccccagctcttgaaggcaagccccggttttatgcatgaccgttatatttgttattttactgcacttatgattgtaggcttgtactgtgcacttaagtgtacgagttgattaaaaccctagatgcatgactcaggttccttttgagatgaatactaatatgctaggtcgagtaactaattatgtcaaaataaggatctcggtagaagtcaaatgatttttctagcactcgcccgaggtcaggaatttgattgtaatcatcttgacaatggaaattatgattgtctatagacatggatccctaggggttgccctgtctacgagatgggaatggaataaggattaatgtgtggtaccgtgtgtaaagcgtttgaaagtactaaacacatatcgagaaatatggtaaatcagtaagcccagtacctgagtgaacctgcccgcggactttatcctcacttgtccttgagacggactctcccatcctagcgatgataggtacaagtgcggccactgcacggtggtagttggtgtcattggagcattgtatgccaaggcagtgaggccctgttctgttgccaaggaatcgatggggacgattgatgtgtgtggggacaaagtgcccctatgtgttgtgtgtttaggtttaccttgcaaggtttaaaactcgattcgaatcgtctgcttctcccagctaatgagacttcttgatccatgctactgcatcgagtaagaagtgaaattaggtttcatgatataaagtgttgcttgcaataaatgtttattaccatgtatgcttagaagaatactaacctagtctaaagaatattctagaacttgaaagctaaaatttgactcagactcagctagtgctttggcaaaccaaacccctcacccaaatagcttcatggtctagaggtagaggagtagactcctcacaccgggtaaatctagctaagtattagtatactcagtcttggttgtggcataattttagaggtacttcattgaatgacatggttgctggagtgatttggccaccatcctgcattgggttggacggtcgagtgagatgctgcctcagtaggaaaggaccaggaagagtagtgggccaggcttcaccctactccttagttatcatggttagttacttgtggcatgattctgcttagcttactacgagaacccttaagcatctccaatattaacttaaatcagtgccctatcttgaaatagaaaactcaactcataaaaaaaaactcgccttattttataaatttttatcaaaagaatatatgacacaccataaagtacccaaatatactaaatatcatattattgccatatccatgttttgtacattattcttaacctttttctgaccaacagtttaatttattttctagagtatatgatttaaggtttaactgttgaagctcaagttatctataatgtactaaacactttaaattatatagtattttaaactttggagcattattttgatggagatgctgctatattaatatattataaataaatgttaccgtcaccatagagcaaattttagcttttaatgtttgaagactaaaaaacgtactggattcttgttattccgactcgcatccgagatgaatgaacaataaaagaattccagcaccttgatccgtccgatccgatgtgcgtccaccgtccatccttccacaaaaccccagacagaagcaaagctccaccactccgccacgtcacgtccgccggctgcctctgagatttgcctccccgattggtcgaatccgccatttactcggctcgccacccaccctctcttgctacctactagccactgcactacgcctccctgtctgtctgtctgtccctggaccctggcgagcgcatgccacaatgccggctataaaacgaagcccggagctccatccccc

SEQ ID NO: 10 - DNA, 3000 bp, Zea mays
  source 1..3000: mol_type = genomic DNA
tatatttgttggttcaatctgatcatgtcaatccatctatgaaagaactctactcgtgatgtgtgaatgaaattgagctgacactatgtgttttgaactagtgtgttcgagtaaagggtccagactctttagctggggttggtaacaccaaccgagagtaatgtagactctttagtctgggttagtgttaccaacctaaatcaaaggatacagactctttagttagggttactaacaccaacaatatagaccggtgatcaatactcatataagggcttgtttggtgacaagtgaattgatatggattggagaggattaaggagaaaataaactaattttctcctcgattccctccaatctctttgataagggaattgagaaggattgaagggaattaaggggagaatgaactaattccctcgtcaattcactggtcactaaaccagccttatgtcttgtttgagagcaataacaccggaggagataggagtagataaaatccatttgaatagtaaagggattttagttccttcaatctccttcgatattcttctcccaaacaagccctcgagagctagtttgagagtaataaaatcggaggagattagatgtgctaaaataccataaaaggaagtgattttagcatatccggtttttttgcttgcaaactagccctaaaagttgagattttctcatacggttggggagacggtctaaagtttatttttgatatctcaaaacatacctcctgattggttaccggtagctacagggccacgctcccaacgcatgcagaccaacgagtgtaaaatggtttcgcctccacctggacgtacatgctcgatcgctgtcgtgcaggtgcagccaactaagggtctatttgttttgctctcaatccatatggattgaatgtgtttcaatcctaaacaagtcaaaatctttcataattttatccaatcttatccgatccacatatgataggaatcaccggacaagccctaacgagaaacacggcgcggtgaacgcttgtgaggagccctgggccttggagctggacaaccaatcacagagatagtgttgacgaggcaacctgcagatagcaccattttatcggaggagccgtaccggtggtgaggatggaggtgcgatggcgtactatgcgacgtgtgtactaccacctgcatgcggcgaggagaggggaaagggcacacaacagaagcgcacaagcggatggcgtcaaacaggcacgctgtactgtacgcagcggtagtagccagtactagctagctgggtctctgcatgcatccatcggcatcggcgcgcggcgcccgcgacggcgacggcgacggaggcgagcgagatcgatcgagtcggtatgtggttgcattcgtccggcccggagagaaagacaaggagaagaagcgcggcacagggcgcggtgtctgtgtatgtctctgtctcgggtcaggtcgatcaggtacgtcgatcgacgatcagggttcagggtaggtaggtggtggtccacgtacgtcgtcgtcctcgacctcgatctaccacaaagcaatgagaaagcaagggggagtagtagtgcgcggcgcactggatcccgtacgcgtgcatgctaatgctatgctgtgcgcacacgcaccgggccgtacgtggtcacgcacgccgcgcggccgggacatgccatatggcctttgtcccgcgcaggctctctggcagtctggctcgaggtggtcgaggccggggcgggcgggcgggcggcggcggcgggcgagctgggtgtcgcggtcgcgggcggacagaggggcggctgtgctgtgtgtgtgtgttccgcgcctggccgctgtgtgtcctggcgactggcgtggcggctgacgaattcgcgggacgtggcgttgtcggctccgtgtcggcggccgaaccaccacgaatcactgacgtatctcgtctcctctctcctctagactcccacgatacggccaacgaagtgtatgtacatatatacccatggtcatatggcaacaaacgccaacgccagcagagcactgcccggcggcctttttcccatctctctctctctctctgatggcctgatggggtgtgcatgcctgactgactgatagatagatagatggtcaggtccgtctgatcctcatcggcctagctcaccccacgcgaaaaaagccactgctggctggcgcccagttgcgcttgcaacagtcactttaacgagccccgtccttgcgtttgccctcctcgctctgcccctgccgccgctgcgtggtggtgctggtgcatgaggcaggcgtcgtactagtgcatgcaatgcaaccgtaggagtgcgttgcgtaccctggtctgtccctgcggcctggcctgccgcccttgttcgttgcggatgcggggggtgccgggtgggtactgtactgtactactgggtagagagaagtagatatagatagatagagagagagagaggtcggtcaccccgggcgcgggacacagcctctgcgaaaaagcgatccatgtcgcgcctagctttgacccggaacggatcccccaaccaggaaccagcagagcaggagggccaggccaccacctctcgccattccattcccggtcctagctagtcctgttctgttcctgtagcagtagctacggtactacgagtcctcctcgacgtcccaggcactactccactccacgcagcagcaggcagcgagcatctctcgaccagatgcatacaagctacaccctcctcggctccgatcctacccatgccggcccaggcggcctataaaagcgcacccccggcccgtcttcctcccactgcatgcccattgcccccccggccttcgccgtgccaacgacacacctcatcaccggccggaacattccacgaccgaagaaaccagtccctagctagtccacgcacgaccaacaaggcaggcgagcgacgacagtccaaagcctccaagaagaagaagaacgaag

SEQ ID NO: 11 - DNA, 4106 bp, Arabidopsis thaliana
  source 1..4106: mol_type = genomic DNA
acaccaataaaaatacacagcaataaaatcgctacgtatatatatatataatatgtattatctattacaagatagtaatagagtatagcaagttgtatcatctaacaaactatgcgaataaaatttgaacattgtgacatgtagatgtagtgtaatttagctaagtgcttatcatcagtaacatagaccgacttaactttttacgaaaaaaaaaaagtaacatagaccgaaaaaatgcatatcgtaaatttaatggaaaacacaatttacgataagtaaaaaacaaaaagaaattacgataagtcgagaaaaatgcaacaaattgagataaagtattgataaaaccatgaaagtgtcggcgtatgtaaatgcggtgattaatgtgatcattagagcgtgtgtgttaaacgcggcggttttagtggagattgatcagctgataacactcttaccgggacgaatctaattccatattcatggcttgttaaaacctaagacatacgcaatctctaatttgctagtatagttagttctatattatttttcgactaataatgtaaacatatgattattaagtcgcaaaaagagtgcttaacaaccaaaaagtggattaattaacttggtgggaaaagttacaaaacctttaatgattactctttgtaccaagaatagtggcgaagcactataagagcagagaaaagaagctcaataatgtactaaaagttgtagatttttacagcttaaatacaccaaaattaatagaaaagttggtaattttttaattcatggctactgatttagattttagaaaacaatagtagtatcattgtcacatcttaaacacacaataggtatgttttaaatcaaaggccgtagttaatttgtcaaaaatgtatgcatttggtatttggatgtctccgaaaggatggatatatggacttgttagataatttcatacctcagtatcaatagtcatggagcccaaattgctcaaaaacatatttttaattccaagactttgatgaagacgtaataatgagtccaatgggccatcagatacaatgttcggaatttaacgggtttgttagttataagtattgggcttgacctatctggttcaatgatatgtaggaacaacccaatttgcaaagctttattaaaagactctttagttgtcgtcaaggtttaacttgtagtagttggtaagaaattctacgtgaaataggcaacattacaaaaacaaaaatcaattcgaaatcatacaaaacgaaaccaagtagtaaccaactacactattatgacattaatgattagacattcccaaatcatacaagttcctgtcatgaaggaaacaatggtccgtatttgcaaacgattacaaaaattcaaaccaaaaatgaaaaaacgagttaaattatttggtttataaaaatagtaatgtcaacagaagactagattgggaaacctgaagcgaacagagcttttaaaaacgagtttgaacggctgggatcatttggtacaatacccaccgtaagtttgtttaccctagggatgcaagccaaaggcccaaatcagttactacttactgctacaaccatcgtctcagctttttgtctcagctttttactaatgaagcatacaatttcttgggcatgtcacatctcgacacgtgtccactattctcttctcttattggctactcgttcgtaggcttctgttaatagatgatctctctataactctaacagtcttttctttctctttatttcgttttggtattttaagtttcaaattgaaaataataggaggaaaagtctagttttaaatattgtttttttacaagtgaacgtgaaccaatttacctctttttttttatatatcctatcggctaatctggttagtatcggtagaaatgcaccgaggtgctacagagattaatgctagggatagtcagaccgcttgtatttctgactatcaagtaaatctacgcccaactcacatatttcccaaacaaatgtgattttttttttttttttttttttttttttttttttgtaacaaatgtgattttgttttcaaggaaaatagaacttacgtttgggaatttcacccttcactaaagcttccttctgccattagaccacaaaggcttgggcaatttaccatttttgtaaaagtagaaaacaaaatgcctaaaatgttcatacttcattacatcaacaaggttatgcccacgatatagaggcatgtaacatttatatatatagtggaagaagcctacgagctttattaataagtataaactctgattattaggtaaataaattacttaaaacgattactcaactgacaaaaccgtagttgaataataaggttactatgaataccgattgaatattgcaaagccggaattgaaaaatatataacagatcaaatgttcaagtgtggtcataattctcacataggtcatatagctgaacccatgcatctatttactagtctatagaaagtactagagacgcatacagctgaacctactctattcttttattaattttggttctcgtggatacaaaattcctccaacatttattagaacgaataaaaccaatatgatgatgattagttattggtaaacatataaacgttgagtaaacttcaaaatagattgaagtactattaagacttgcattttttccccttgggttatattcttgaatcgtttcgaagtattttaactttcaagaatagaaggttcctcaactataaacaattacattaatcaaaaccatttctatgtaaacaacataatttttgtatattttagtcttccccaaaagtttgaccgatagggcggtttagaccgtatagtacgactgtacaacaaaaaggactctggagacctaaagatccaaaactatgcaaaataaagatacggtcggaccaatttaatctaacaaaaccaaatccttatactaaactatttaccgatacatttccatataacacagtacacacaattaaatcaaacattattggaagaacaagatagaatattggcttaatctcgaacgattagagttatcctagagcctcggagcttttgtcacatataatataaactatggtatatataaacatgactctcatttgtatttatcgcaaggtacaattccaccaatttttttcgtcccactcatacagctttaattgtgaaatcaatccataaaaaaccaacatgtgacatggtctctataactataactataagatagtaaaaaattcacatcaacataaaagaaaaccaatcatattggctaaaaaaaactaacggtcgaaaaacgtataaccacaaaaccaaaccggtccaaccggtgtccccaatcactatcaaagcattaactaactttcacaaggaaaagcatagttcagtttctctacatcgcttcccatcctcttaaccctgtttactcgaatcatccaccgttggatcaaacacgcgctacaaatctagcgcgtgaccgaggtttttacacagtggaatattaccatgcattggaaagcggcgtctacaacaaacggcgggtcatgtcaccgtcaaaatcaacctttcttaattcctaacgccgttacttatctccgtttactaaaaatgttaatgcgtgtgagagtgaagatcatatactaattagaagtggctaatgttttaacgtgacattattatcatagttaatggttcgatcagagttttaagtagtaaatgatataagtgtgtgtatataattgcatacatatatactctcacactctgacagatttgtcgtggtcttagtattctctttcatggctagttatatagggctctagtacattatctctctctccccatttctctgtctctctcttctttaacatctctggctctgcatttcttgcagttcacgctaatcacctgttttggtacccttttctctctgcaacaatagcagaacaagcctgaagtttcgttcttttgtgttttggtttcctctgctttgttgttattgaatcaaaacttcaacgagcttcttggcttattaccccaaaaggaaagaagaataagaaga

SEQ ID NO: 12 - DNA, 2000 bp, Glycine max
  source 1..2000: mol_type = genomic DNA
caaacggagttcataaatgtgctggaagattcattatctacgaatattaaaggatatccatccaatccaaccaatttattattttttttatgtttataactagatggattgggtgattagtaaatgcatggattgaatagtcactcctataagtattctaagattactttagaaaatggcatatttaatattggcctgtcattacagcataaatggatgcaggtatatatataatgcggtgacctgtacaaacaaatgttcacatcagcacatacacttcaaaatcaataggaaaaactgtaaagatgcttacacaaaaatatcgaagatgtttacatatttcattacaatttgtttattttttctttcgtgtgtgtgaacaaaataaatgatctgcacatgtatcatggttatagtaacaagtcccagctcccaagggtagtagctgcttaaagacacacggaaaccctcgaggataatggtggagagagagtgttgaaatttcaaccatctcacttttttcttctaccttattctttagccgaaaattaagttctaactttaaaatacagatttgcttcagaagttgattttttaggtttttttttttctggaagaccaatattaatttgtgatagttagttaaaaaaattatattatgttttgtgatagttagttgaaaaaattatattgtgttttgctactaatttactttcaaaatataaatatccaaatatatatataaatcttatcacttgaaaatagatggtgggtatatggactcaaatctatggatcggtctgtttaacccacgaattttgcagataacaagcctttttttaaaagaaaattcgcataactatatccaagtttttcttagttcggtccattaagctagcacatctagtagtctttatccatggacatattattaatttgttatgttaaaattttgatttttaatctagtggtatattttagtatgatagattttaacttaaaaaatgaagtcaattttttattactaattttatacatttttcacttttaaaaaatatttatttcataagaaatatttttaaattactatttgggtccatggatcaatttatttatctgagtttttatgggctagatacaaatcttggaaaaaagccaatttatttcatggattggcttatttgatccgtctaaaatgcaagcttcacaagacaaaccttaaacataccataccatccttttatctatctctacttcaaaatcaatttttgcaacattcactcaatggtgcacttaattaacaaaccctcatccatatacacatatttagagatcaacaatatgattatctatcaaaaatacacaaaatcagtgtgtgtttgggtaggcgttgacaaaaattagttttgaatgaaattgattttataattttttttttgttaaaattgattttgaagtaatattatttatgtttggatgttttattaaaaaattaagttatgaataaaatgaagtacataattttggaccaaaaattattcaaaattatttcaacccaaaattaattctgtatccaaagtcaattttaaatttttctttgatgtgaaactaaacatgtaaaaatgtatttaaattaaaattaattttagacttataattaattctctgtggtcaatccaaatacacactcacacttagaatgtatgaaatttcaatttttaactttctcatctatgagctgttcctattcctccttcccctcctatgccctcactagggagccagccagccatattccaaaagcccttattatcacacatgggtccctccatagtcaaaataaaaataatatcatgatcactgtttggccataaagagctatacgacacacatggacacagtagtacactgcccaaccaatcacacgtcgacagcacaccgttcccaaactacttcacctttcccaaaccagaaaccaaaaccacttgtcatcaaacccc

SEQ ID NO: 13 - DNA, 2000 bp, Glycine max
  source 1..2000: mol_type = genomic DNA
taaatgatctgcacatgtatcatggttataactagtcccagctcactagggtagtagttgcttaaagacacacggaaacatttaaggataattgtggagagagagtgttgaaatttcaatcatctctctctctaccacggtatacttcaataaaaaaaattaaggactaattttaaaatacagagatttattctagaggttgattcttttagtgtgtttgattttatgttaatacctcaaacatacatttagaatcccacaaaattagtttggtatcatgtttggattttcaaaagttaattttgtaacgtacgtttgggataaacctaggtttgggtttggatcagaccaacaaatttgtagcttttaaagtgaaattactttttatttgtatatttaggatagaaacatgcatgaagtaaaaatacttttgattagtatatatccaaacaaatatcaacacttttgaacaaaaagggtaatcatgttctattaatattttttttttttgtgataaagaagtagctgaagcttcttatttgacaattaaataattcttctcctatcggattggtccatttttaaagtaaaatgatacctcaaattgtgactatatacacaagccgtgaatcaaatcttagaccttggttaaaagactcaagtgctaagtcatttgtaccaataacttttggtataagtaaactcattttttacaacatacttttgattcaaacttatgttcgcaaactttatacaaatatgcacttagtgtgtttggatatttcctagaatgaattttgagtgccaaatcaaggtggaaatattgcaaaggttaaagtaaatatttgttactttgaccttttcaagttaccatgttattgataaagagtatttatcgatttttattaatgattaatctttgtcaaaaaatttagttagttagttttagcgggattttttcctcctaattatatttttttcatccacaatacttgaatttgacaccttcattaaaaggattaagtctgtattactcagattgttactttgcctgctttaagttgattttgataagattgaaagctaatccaaatatatattttcacattaaaaattttattgcatttaaaattaattggcttaaaaataaattttgaattatatatatatattgaatttgttataacttttagcaagaaaaaatatttaaacacagaccacattatctcaaaataaattttaattaaaattattttattcaaaaatcaatttttatcaatattcacccaaacacacaaacttaatttaaaaaaaaatcctcatttatattagagatccacaatatagttatctatatatcaaccatacacaaaatcataacgtaagaaatttcaattttcaaccatctcatgtgatctatgcatgctattattcctcctacccctcctattccctcactagggagccagccatattccaaaagctcttaacacacatgggtccctccatagtcataataaaaataatatcatcatcactgtgtggccataaagagctacaagacacacatggacacagtagtacactgcccaaccaatcacacgtcgacagcacaacgatcccaaactacttcacctttcccaaaccagaaaccaaaaccacttgtcaccaaaccccagtcccagatagttttctcaccatttccgtattttacttcacctttagtgggtagtggttacatgatatcagcacactttcactcactgtgctttcccgtgacacgcatctatacttgtccagaagaagtaagatccatgaactaacattcccacacgctcgcttcaccatatttgccacccttttaacctcacttcttagggttattttgctcccttttccttgtttgggaattgggtttggtttggtttttcttggttgtaaaaggtaaagaagaactaattggttaagttactatatatctagctagggtatataatatt

SEQ ID NO: 14 - DNA, 2000 bp, Glycine max
  source 1..2000: mol_type = genomic DNA
agccgtttgagtaggctttattcaaaattcagcatctgcgttttttttttgcattggtgtatatagtaaaaccaaatcctctatttcccaggtcaattcgttttggtttaaggagggtgtattagattgggattttgagagaatattttgacaaaaacaatcttgaagattttaaaagattatgtgggattgtattgattttgtgggattttaaaagattttttttaaaagactttttacaatcaggattcttaacccaaaattttaatggatttctaacacagatttttaagatttcaaagtactttatggattcttaagattttgaaagattagtacattttaaacaaaaaaataacgaaagttacaaaagtgaatgaaaaagatgataaataaattataatgtttgaataaagaaaataaaaacgatagaaattttaaactttttaataacaaagtcattattgcctaaaaaaataataacaaagtgattctcacttcatgttcgcctaattattaacatttctccacatatctgaacctatattagtcctccatatattagcatcttctcgttcctgctcttgtgtttgaacaatggattcatgatcattgtcttcgtaatttggtaacactgaagatgaagatgaaaactcgtcagtaggttccactggaaattcatcagaacgacattctttgcgaagaaaattatgaagtgctgcacatgccaacacaagctctgcttgtgttttaaatagaaatggaggtgctgacttaaaaattgtgaaccgcgatttaaaaataccaaatatcctctcaatcaaaaaacaattaaaaaattattaacacaataataataataataacacgctgtcaaaaaaataataataacacattaataattaacattttaataataacacaagaaaataataataataataatggacgttgtaaaaaaacaagttgaagaaacagaaaaaaaaaagagttttttattttgatttgcatatatagtactaaaaaaaaagtaatatgaaatcttgatgcatatcaattgtcattcttttgatgcatattcattgcctggacgttgaaaacaatatgaatacgaaatcttgaagcatatacaacacccttttcttttgatttgcgtaatatacatatagtatgaacaatattgactatcgattgtcattaaaaaaataggtaaaattgattgaaaagttgtaagagaatgaacctggttgtggtttgtgtcttgttcggcaggagaagaaaaagtctttgaaagattatgaaaagtctcaagggtttgggtgagattgttggaggagtattttatgtgtatttctaattaaaaagtctctcaaaatccattccacaaaagaatccatcaaaatctatttacttttgaataacaaaagacattttaaaagtggtaagaaatctcaattaaataccaacagattttgttgccctgaaaaaaaaatctttattgtcttaattgaataccacaagatttgtttatattttataaaagttttgattgaataccacaagactttttaatcataaaaaagtcttttaaaatcctttgaaatcttataagaaaattgttaataaaaaagttatacgaaaattgaaactgtatagcagtgtttattacttggtgaacatcagaacaactgaacaagaagggaaataacagcgaggaacttaggttgagaattaatacggggaaatatttaacacataatgcatttagggtgcatcaagtaaaagaacgaggtgcgttgaataattaacacagtatggctttatacactgtggcacggaaagcccaaatacgattaaggtccaataaaaattccaattctttaaataaatattaaaaaagtgaatttgaatctacacaaatcccgcccatgcgaaatttgaaattcaaaacggaccgttatattttttgaacgtggacagtagagttatccaggaaaaaa

SEQ ID NO: 15 - DNA, 2100 bp, Glycine max
  source 1..2100: mol_type = genomic DNA
gctacttactttcaatatactacatatagtttggttgtaagttaatagaaattgctttattattataatctttggtttgagcatgaaatggtatctttattttaaaatgaaccgaattgataagagaagattaattgagtgtgttaacggtgaaaattatatttatttctcgaaagaaaaatattttcaagatagtaatataattttcagtaattgattatattatcaaataaatatttaaaaagtgagatataacatttattttttattatgagttctttttagttaaataagtaaatttatagtttttaatttaatagaaaattgtctaatatttaatataaattataattataattttgataaatatgttcatttgaattataataagattgtaattcataagataatagaataactttagataaaaatatttttgatttaatgataattagtttctaaataatttcaatgataataaaaaataagaattgaaaaagtttaaattttgagaagaaatgaaaatgagaaagaataaaggattaatgaatgttcagtaaaataaacaaacatgagttcataaaagttacctattgaacatttgtcaatttctatgatatgaatgttggtgttatgagaaattaagtgattgtgtatcaatcctaaaattaacagtgaattattttatttatagatttctaacaccaactgttgaattatatgatgttgatacgtgtataaaaatatttctaaatacatgctaataactatcttggtgtaactcctccgcaaccatctcaaatggttacttcaatttgtcaaaaaaaatttactttcagtgctttaccatctcacgtgggaatgaagttatggaaaacaatatttttaaacgaaataagttttttcaattacataatcttccctcgaatttctcctgtccaattcccgtttgaatgcctccaatcctatctaaaacaaacaaactctgtcactcattccatttaaaactgtgatttctataataagcgagtttccgtgttggctgcctccgcattgaacacaatgttataaccttgggattgaagattgatctgcatatgatatatatagctatatatacccatgttatattgctcgtagctaggtttaagcgttaaatgaaagtgaaagagcaatttaaagttcaaacatcaaaatgctttagcttgtcgagccccactaaattggaaaaaagttaacgttgtcgaggccttgtagtgttgtcatttcgaatatttcagtacgccttaatagcaaattttgaagtactcatttgctctccttaattatctttttagttttttttttcttgtttaccttaattaaccatcatactggtgtgcgatgtactcattaactttacccatgactaatcacaaaagttttaggaaattaatttatattagtatgtcattttaaatatttaaattgattattattaattatattttatttttactttattttgtactagaaacacaaaatatgttataatcttgcatatacaatgtcaagtatttattattaattttacattatgaaacttaatatacgtcactttttcacttttaatcttacatattttaaatatttcaacgcatcaataataactatatttgatttaagagaaatgatatttgtacaaccaacagagaaaaagagaaaaaaaaaaagagaaaattttgaattttaaatataataaatgataagatgtggaagagataaataaatacaaaaaataatattgtataaattatttataaattattatatgtgcaagagtgaaatctgtatcttagccgaagatatttttctaggacgatcatcttaaattttaattacatctctctatggtcaaattaattctatgcatgttaatatggctaactttatgattctacgctgctatttagatatagcattattttattctcttgatgatattgaaaaatatatatcttgtcaatgtcatttaatgtttttcccttgcttttgttggacttatattgcagataaagtttgagcgtccgtcgctcctattctctagcttccaagaaaaca

SEQ ID NO: 16 - DNA, 2000 bp, Glycine max
  source 1..2000: mol_type = genomic DNA
tctcttcttcttttatattgcattatttagaagtactggagcgatataaaatagtagaatatgtgattcattttattcatctttaattttttaaaaaattaaaaattaaaagcttaataggacagactcttccaggttacccacgggatttttgcatatcagagatgacctagatcgtattttcattttcatgggactatgcattaagacagggattatcaatatcgtgatattataatttacatggtcgaagtgatagaagctttcacaatcatttaacattgtacatttgtacgttatttcatactaatacaaagtactacgatttctcttaaacactagatcgcgcacttattttttaagacaatttattcttcttcattcataatttcacccttcattattttttacgaaccgtattcaagtttcttcagtagttattagtaatgttacttatgagattacgagctttggaaaatttaaagaagatttactcgattacctcttccgttttaaaaaacaatctgataattttacttattaagttgcaccaaaacatctttttatcttcttccacagcaaaacaattctgatcgaccctaataagagactttcgtgacatcaaattctttgtccgaatatttaatacaatgcaaactagaacttgtgttaaaattaaaaatattaaattaatttttttaaattgtaatataatattcctcggtttattaggaagaaatataaatatctccaattagaggagtactagaaacattatctctaacatatatccctttaaatatattttctcttaccggttaaaatttactaaattgtgagatagaataattaaatatgatattcaagtaccaacaagttattcatcaaaatagtattgaccttagttttctagcaagatgatactgtacaaagatcatgttgacaaaaaaatatatagagttcacataattttgttatttttaataaatttcaactaatagaagaaattatgttagaaaaaaaaacttgtgcactaataatgtacataatctttacaatctttacaatcaaggtaaatttcttatcttttaaaataattatattaaaataattaaataataatttgtgattgaatgaaagtataaattattttacactgaaaatagatattaaacttgttgaaaatgtgtttaagagcgtgtttctaatatttagtttttcattacttttgattgagataacaatgcccttcataaataatctttcgtgagttggtataactatcataaaccagtatagcacaaaagagtgctaagaacacatccgctaataattttttttttaaacacaatttttttcattagttttaatttatttaaggttataaaattaaaaaaatatcattaaatatgaggtgagatttaaaaaattaatatttttaataattttaatcaataaacaaaatatatttaaaagagtgtgttgttaatattttttaatctgtatattttcacaactatacacagattttgaacacaaaattattaattaaattgaaaatttcataatagttgattcacctttgcataacataatattacactatctaacaaactcaacatctataactaccaaacataactccttccaagtattttttgtatgaagcaagcatctcttagctaaatataagaactaatttcttaagatattaagatttatctaagagaatgatgtttttctataggcattaaccaaaaatcaaattcttaatcacatacttaaagaatactttatttacaaatcatatcatattataaattaaactagaagattttcatagcagtttatttacgtttgtttagtgaaaaataccattcattaaattcccgattttataaaatattagcaagatctagaaaaaagatagtatcacgttacgttacattacactatttaacaacacagcttccataactaccagacatagctccctccaa

SEQ ID NO: 17 - DNA, 2300 bp, Glycine max
  source 1..2300: mol_type = genomic DNA
actttctatatatatactgcaagagataattatttccgacagctatttatgtattttgtacagctaatatcaagttaggctgatttctccgcttcccctttgtttgaaagttttttcgtgtttttttttttttttacttagccaataaaatagtgacctacgtacgcgtgtctttattagtcaaatgctactataatcttaatgtctttattagtcaaatactaccataagtaaacggaatgtaacaagtagacgtgtccaaaaatcatatttctagattaaataaaaaagacagtaaaaccctctcaaacttgtggcacttgagcaataacctaaacttagcttcgcctacacacaacgcatttggaaaaatcacaattgagatatgagaaaatatgattttaagactaagtgtgtgtttgaattacaaatcaagacaatctgaaatggaggttggagaaacaccaccaccgtacccatgcttatgcacattgaagcaatgctgggttttgtgcggccgcaagtttatcagaaaaataaacacttgcgctaaactggcagtttgaatagaggtgaataaaaattgtttttactgttataatctttgaatttagcatgaagtcgtaattgaatatattactttactttaaaatagaccaatcaataggagaatagaagattaattgagtgtaagatagaagcttaaaatacctcttgattacactagaaaaaaattcttcttgttaatatattttcaaatgattaactaattaacttatttaaagtgccaagaagtcactggcggcataaaatttccgcaagtagcgttcagaaccaatacttgtaaatttggtgtaaattttcttaccgtaaaacaagttctggaggcataatttgcagaaagaaaacaactttctcctgtccaatttccgattaaatgcctcctatcctatttagaaacaaacaaactctgtcactcgttccatttaaaactgtgatttctataataagcaacacaacgttttatccttggggttacagattgctctgcatatgatatatatagctatatacacccaggttatattgctcgtagctaggtttaggcgttattaaatgaaagtgaaagagcaatttaaagttcaaaaataaaaaagctttagcttgtaacgccccactaaattggaaaaaagttaacgttgtcgaggccttgtagtgttgtcatttcgaatagttcagtacgccttagtagcaatttattttttatcataagaaaaattataattaaggacccatcaatcgggtgaagttacatccaacatgcatggctagcagagacggcttaatagcaaattttgaagtgctcatttgttctccttaattatcttaattttcagttcttcttctaattcaccttaattaaccatcataattgttgcgaaataataaattgaactaatcactgaagttttagaaaactactttatatgtcattttaaatattttaaattattattgttatatatatttttttactttattctgtactattaacataacatcatgtcataatctcgcataatgtcgatatttattattagtcttacgctaggaaaattaatatcattaattatattttaaatatttaatgcattaataataattaacgattagatatttgtacaacaacgagagaaaaagaaaattctgaatttcaaatgtgataaataatagggtctaagagagggatagacacaaaaataatattgtattaattgtcgtatgtatattatatctcaaaatcactttttatttttgaacagcataaataatataatattaaattttaattatatttatattcataaaataagatttttatttttatttttttagtatgtatgtacaaactgcttatattttttaaaaaatagtttttttcttattttaagaaaaaaaaaatatctacttaaaaaaacgtttgttttaaagttaatttttttaagttgaaataaactaatctgattatttcccaacacactataatattctaattattaattataaaattgccatacttttaaattaatgcattaaatatatataacaattggatttaatagggaatgcattcacgatttttatatttttagtccttatagtttttgaaactgatttttttagtccttatagtttacactttaattcttttctagtccctgtagtttaaaagtagtttttttagtccttatattttgtattttaattcttttttagttcatatagtttgaaagtgatat

SEQ ID NO: 18 - DNA, 2000 bp, Glycine max
  source 1..2000: mol_type = genomic DNA
gaaatagatggaataaaaaaaaaggaattcaaaaggattcaattcctacttcaaccccaatttcctatctcctctttgtggcccttcttctcccttatccctcactatgaccaataacaattttttcattgataatatatataaaacaaaccagatgaaagcaccaattgatatataacaaataatagtatactagaaaatacctaaaagactaccattaaagctataaagataataaaataaatattttgttttttcattgataatgaaatggcaggagaaatacactcatataacctactggatgccaacaattaaataatgaacctacaaatgtcactctccagctttagggaaattaaaatgaaatcgaattgacattataaatactaaaacaatattcaaatactaatgttattttaaattttatcaaaataaaaagatgattgaatgattcatatatattttagtatatttttaaaaatatataatatgttgatttcaatctatatgaattcaacattaaataattctaaaaaagatataaatttttgtatacaaatctatatttaagaaacttttaatctagatgtcgattttaaaaaatattatttaattaaaaaatattagatggtgtaataattaatcaaaattatatcaagataatctgattcctttctatacacacataatattatttcatccttagtccctaatattttcaattctcattttgttaccagacttgtaccgaacaaaaacaaaatatctaatcatagttttcattcaacaaaaatgatttttaactcaatttgaaacacttttcattcatttttaaaactaagaaaaaatttgtgatttatttttataattttgaaaaacattctaccatcattattattgtttctactaccatcattattaatgttactactatcaccttcctagttataaccgtaagcattatatatttttattattattgttattatgttattttgttaatatatttatttttgttctaaaaaattattattttttcatatctttcactatttttgttattattttagcaagtttgattattttttattttaaatatttttatgtgtcactttttatatcacattatttaacagtgtgaatcgataaaaaatataataattatctttaatttgtaagaattttttcaaaattaaaactgattttagttcttgaaaaatgtaaaactaaaaatgaaaaccacctaacggggccttagtaattaggacatggtctccctggttacccacgggattttttcacatcaaagaagacctggtattttcattttcatgagatttttgcatatcgaacaaggcattaagacaggggttgtcattgtcgtgatagtataatttacatggtcgaagtgatagaaactttaaccatcatttaccttgtaccttactataacacaaaatactacgatttccaaacactagatcgcgcgcttatgttttcagacacattattcttcttcattcataaataaatttgcagctagtatatgataattgtaccaatttatgtaagttttttacaaaggacattcttatctcaataaaaaactaaatgtttaaaatattctctagcacattttttaaatacattttgtctaattaattaaaattaaaagaggatataaaaaatatgctgctaacatcttgaacatttccgcaatcaataatttctcaatctatctgaatatttttgcaactgtatacaaaaatctcagaacagaaaattattgattaaactggaagaatttaataacatttgattcacgtttgtttagtgattaaaaaatcataacattacactatctaacaaatgcagcatccataactaccaaacattaaacaagagaaacagacaaagtccaataatcacagagacacgcagtgacaaagaaaagaaagagggaacggtaaagagaaaggtgtctctgtcatctcaaa

SEQ ID NO: 19 - DNA, 2000 bp, Glycine max
  source 1..2000: mol_type = genomic DNA
aaaaagaattaaactcttgcatcttaaaattaagttattaaacaatttttttttaaaaaactatactagcttgtctgaaaaataattctgagacccataataattttttcttatcatgattgtgcaacgaaagttaaattatgaccaaacttgcgtttcaactaaaaatcaaagagggcaaaatgtgcaaaattggataaaaatgattgtcagctgcagacgaaaggggagaaaagaagcaagcaaaaaacaaaagatcacaaaattgaacaataatgacaatacctcgcattgaagtagttcaatacatattctgagtatgactcgcgagtacatgtatctcatgtttcagtttttaaccacagctggatgccatggacacttttaattctattcttttaaatgagatacctggcctatcaattttaagggtaaattaaaactcagaaatcaacatactaaaaaattgcatgcatgtccgcaaaagaaaatgtttaaggcagtatatattcactagtgcacaaagttttgaccttaaatattgaaatagtgaccatacgagtgtaatgtttcagatatatataatgctaattatggactaattaattaaaatagaggattgctttcaataggaaaatagtagtgtaatggaaaccggaaagtgaactagaaagaaatgttaaaaatggtctagggaaaaagttaaggagaaaatggattatacataatgtcaaaaattttataccgctgattataatttatatattatgtaggaaaaatttattaaattttaaaataattaaaattataatttatgattgaatgatattataaaacaattttacacaatcaatttataaatattaaacttaaaagataaacctaaaaaatactatttttctttatcataatattagtataaaataattctactaaaaaaaataattaatatttataaaaatacatataaaataaatattttttttataacataatttattttatatataaaaattaatcaaatattttaactttggattacttaattaaaaaatgattcctaaaaaagaccatttgaatattatttggtggacgaagggatggaccagagttcaatactcgtgggttttggcggcgtcaatgagaggatgcacgtaatgttcgggtccgtacggcattgccactgtttaaaccatcggacacttgaaagctcaaagcagtgactgtgatgagcagctcgcaatagagaatccaaaattcccaaaagcaaaaacaaaacccaaaaagcaaaagagatatcattgtgtggctgttagataccacagacagcaacatggtaactgaccacccaatccgggctcgacagcgcaacgttagcaaaaccgccctggttttttcccaaaccagaaaccactttatcgccaaaccccaatgtgattagagtgttgctaggtgcacctactgttattgctggtgcacccaaccttctttgataatgataaaattatccgtcttctttttccctttacggatcaagttgatccgcaagttgatttttaagacttacagatcaagttaatcggtaagtcttttatggatcaacttgatccgtaagtcttaaaaattaatttaccaatcaacttgatccgtaagaagcttgcagatcaagttgatccataaaagacttaaaaatcaatttgcggatcaacttgatccgtaagaagcttacgaatcaagttgatccgtaaaagacttacggatcaacttgatccgtaagtcttaacaatctacttgcggatcaacttgatccgtaagaaacttgcagatcatctatatcaactgtagatcaactaaaacatatgcggatcgactcattacatatgtggaccaacctgaatgagctaggtgcacaaaaatatttttaaaaatacatagggatatttttgtcttttcatgttaagtgttaggtgcccaacaacaataatgctgggtgcacttag

SEQ ID NO: 20 - DNA, 2100 bp, Glycine max
  source 1..2100: mol_type = genomic DNA
aaaaagttggagtcatatgtggaagagaacatctgcagattggaaaccaaatttcagtgtggtgaaaaccctagctattatgcagtgagtaaaaagatcacaattataacacagaggtagcttctctgctcctacttaacggtccaagtacagtcctactactcttatataatcattttctgagattccccatctctatctttgaaaggagactactagctgctaagtactaagtaccattaaagaattattattgttattattattttttgttttttttaagtatatttttaaaaaaagaaaaagagaagtatgtatgttgttgaggaaagtgaggacaacagaaagggagatttagtttaggccaatgaagtacttagatcataatttcatttattttgtgaatacaactacaatattaccaatctgggttagggcagaggcagaaaatctagctcaagtatcaggacagacaccgatcaaaaggacacaaataattttttggtttgtaccatattcaaaatattactagcatattcaaggccatcaacttcgagaagaatgttatacaaaaattaagtgacatacatcaacataagtacatatacttcacggcgaatttattaattatggtaaagacgaattaaattacataaaaagaatgccaaaggaagttagaaagagaagagtggtacatatggaaaaaaattgggaaaaaaacaagtgcttactaagggatagtttgatttagcttttaatttagaaggtttattaattgaactttttaagggtagttatacaattgaaaaagaaaacagccctaactttgtggatctagttttgatgggttttatccgaaaggtaatgaacttttgaagttgagccgtacaggaaatgtaaaaggtaggtgagaagctgtaatagcttctcagctgtttgttctgtaaaaactaaaccgcaaatatacaaactcgtcaggaaaaataaaattacattttttcattttctgaaaagctgaaagtagtacaaattgactaaaaacttggtgttcttttttaatgcgcccacattacttcatttcaatcccgaaaaaagctcattcatctctgacagcgattgcagcttccctgggatacatatgtgtatgtgtactcgttatagtatttaagtacatgaatacgtaggtgcttaaaaaaacatttagttatttacatgcatattataattctatctgtcactttaattaattcgtcaaaaattgatttcattccctagactttttaacggttaatcttgtacaatattttgcttttaataaggaaaaatgaatgatataagtaaaaaataatgcgcaaattgaatattttaccacaaaacaattgttaagccagtactattgatatgtaaccttgtttgtcaaattaagaatagaaattatttttttaaaatacaatatatatatatatatatatatatatatatatatatatatatatatatatgtatgtgtgtgtgtgtgtgtataattactttgagattttctaattccagccgcatggtatagtttcaaaaatatggtgccaagtagatgcagtggtagtggcagctagtattcagtttcagaatgccttaaatttgaagtctcacaagaagattgaaaataaatctatttcatggactcaccctccaattgggagctacaaactaaattgtgatggggtagtcaatggtgacttcactaccaaaaaatagcatttttccgacagcaaaaatcccggagttttcaggaattccttgggtcatgttattttctccttttctgctattgaactttgttctgttgtcatggctgaattgtgagccattaaaattggtgtgcagattgcttggagtcgtggtttccaacagtttcaagtagaatcagactcgcaagtagtagccattacattgctcactagtggatgtcctacttaccatccatgccttagtttaagtcaagagatactgaagttccgggagcttggaggtagcattctgtggcatcatgccttaagagaaggaaatcaagtggcttattatttagctaagtttggtttgtcc

SEQ ID NO: 21 - DNA, 2000 bp, Glycine max
  source 1..2000: mol_type = genomic DNA
aaggggtttgaggttaaggaactttcttgggggcacagtggactgtgacaaagccaacggaagtaacttttacttgatgtttgatttggctttgttagttgttgcactttcttaaccattggtctccttagacactctcagctgtctcatggtcattaatttgcaatatggttcgtggcaaaaaaatattgaacttttactttgttatttactctatgcctcgttataattcttgtataagagaaaataattattatttggattttttaatataaaattaattattattttttattcatattctttttaatattaataataaagaataaaatttataatgaattaatgattataaaattattactcttctatctgtttattgttttttttgtctatataaaaattgttattattttaagacggagggagagtattcggtttattatagggtggctttggacgacacctaattaacttgtattgaattctgtgatgtatagatgagtataccactttagaatttgataatatatttataatttgtttgcgtgataaaagaaggctttggaaaaaaaaacatgacgttgaaaaaaaattactttttttgggaaaaaaatctttatgcatatatgatttaaatcattataattctatggtccaaatatatttctctcattttatgctacgtacaaatatattttgtaaattttatatttagatcacatttaaataatcattttaattaaacatttatttttggtatgtaattcttacaaaataaaagaatatttataaatttaattgagaaactctagataaaaaaatttgataaatttctactagaaatttattaaaataaactataaaatacttacacgaaaataatattattttttattataaattattctaaatttccttatagaataattgtgatgattttgtatacataaatacttttactttcaaaattaactattcacctgacaatcaagcttctttcgtgattaattattttggccttcaactgactattttaaaaagcaacccataaaaatctttgtagagaaacaaacaaaagtataataatcagagatcaaccatcaaatcagaaggatattaattaatcacggattcccacgataagaaattaagcatttgaagtaaagtctaaaatgggtaagagaagtgttcaaaacataaagggaagtgcacaatttcgtctaagtcaagatcatggttgaaagttgaaacaggaggagcaaagattgggtcttttgtcatggtttgaagtttgaacaagcacctgtggatgattcccggaggagagtgtctctgatttgaaaggtttgtaaacttgtgttcactggcaacagccggagatttcgtgaaatgacaacattcagacaaggaagccaagaatcttcctacacataaagagtaccaaactgtgcttgaattccattatatccattgaattattgtgtgtttccgtgattgtttgcgaagttgataacggggtttaaggtgtttttttagttttgagtattgaaattttaaaaataataattagtttttagttttagtttaaaaataattaaaattagttttaaataaatttattttgaaagtttcacattatattaaaattagttttaaagaatttttgtatggcagtgacgataatgataataatagtgatgtcaacaatgatgacaatagaaatattagtggtgatcacgatgacaatggtgatagcgaaggtggtgacaatgacgatgtagtcatggtaacagtgttcacggtggcagctaatggtagtgacgttagtagtggtggtggtgatgatggtcgtattagtagtggtggtggtggtgacggtgacgaagttgatattagtggtggtgatagtggttgtagcaatgttagtggtgttggtggcgatgacgatagtgatggtagtaacggtggtggggtgatggtgatagcagtattggtgg

SEQ ID NO: 22 - DNA, 2000 bp, Glycine max
  source 1..2000: mol_type = genomic DNA
aagaagagatataattatgttatttttatgtaagttattgtaggcaaaaattaaaaacaagttttgaattgattataaactatttttaattagttttttcaaatatttatacaagtgtttttataagtaaattggaaataagctcctcaaggcacacttacactcccttctgcttttactctttctggtattcatgacttttcttttccgattcttggggtgtacctataagctatagaacagttgattttaaaaggattttcggtttgctgtttcacttgccatgggtgaatcattagggacaaaacaaatgaaccttgcaaaaaatattgttgggttgaatgaagttatagacctatagatacgttttattatataatttaaaaaagttaggatcgttaattttagtctcaacttaataatatacgtagaatattgtggcggacagtaacaattgaccagttgtgccgttctaaagcgtacataattttaatccccctaaagggatttttatgaagttcaaaatttgttgcccatcttatttgagaaagtggagggatttagctcaccgtggaaatgcaattttgaggataaaatgcaattagaaaatcaataattgagataataactaatttagattttgttaaatatgattttatcataaatttttaacaaaaaaatcaatgattattttagaggaatcaaatttgactaagtgatgtaagcaaaagttgttagtaggattaaaatgcatagacaaaatggtgttagtaaccttgccaattaatacataccggacaaagactctacgggataaaactttttaaaaaaataaaaaaattatgactgaatattttttttaaaaaaaagattaagtctgaatagttaactttttctctctttaaatctgaaatgacgaaacatgaaaagaattgacttttgagagttatgaatcaagaaaatcacaattcacaagtaccattctaagtaaaagaattttactcagctacgtgcaggcaaatgaataaatgtatgatcaaataataaatattttcttatccgtattaataaaagatgagtactagaaggtttacaatagtttatatacatatgtaattaattgagctagatcttattagtaataataaagggtattggtgtttgatctatctgtgactgatttctaaactttttataagtttgcaattatattttcataattgaatagatttaaaacattaatttgtatttacatttcatctactataaaatataaataatagataagataataaattagtaaataatagaaaagaaaatgtaaatataatatgtaataaagagtgactattcatttgtctaaatgataaattaaattcttattagatatttgcataaaaataattcaaattgatagtattataataaatgggttgatgcaagcaccaccttaagttgcacaggacaacttcgattccttgagcttttctttgccataaagagaaataaagtaagaaaatggtgacgtactagaattcatttttcataaattactgatagaatgcttacataggctctatcattttgaatctatcttgtgagttgtgacaagggtctagatggacattttctttattatttttgttttatgcaaaggcatattgtactcttattgagaggctgctacataatatgaaagtattttcgattgcattatcaaaatcagttaacctattcagttttaaccaaataggtcattcaaattgtacctgaaatagattgcttttgggtggaggaagcatatgagagagagaaggaatggggaggcacttgcattatgtttctgatgtcatacaaatggttttggttttgtattttgtttacagaaagatgaatataatcatgcaattgttgttaattggagccaatatttgcatgcttttcttgcctcttcatatcttaaacattagcccctctcaccactttaaagtaacccttcc

SEQ ID NO: 23 - DNA, 2000 bp, Glycine max
  source 1..2000: mol_type = genomic DNA
tgttcctcttcattggcctagcaagctagctagctccttttgtttgattcctcagccagatgttgcaatcttgttattatatatcaaaaagaaactaaactaataaactgttttactcaaatcatatcaacaaaatcttaaaaaagatagatgagaaaagggcttaaaagggttgtgtttgtgtatgcatgtgtctctcaaaagttgtgttgtgaacttgtgagtgaatgaccacttagagagaggagaatggtgaatgagtgatgatgaagatgagggttatatttgagtatatgagagtgtgtgtggtgtggtgcatagaaattagaaagaggttatagaaagaaagaagaaaaagagcataaattttctaaaccgaagagagggaatcccattggagggtacccctgaaaagtcctgtgtgtttggtcactacttgctaaatttggaacctcggcctgccagggttacacacctttatgccatatttctgtcactggtcaagttaacatgacacattcatattttgttctcggtaaagatccacttattaactctcagatttctctctctctctggcacagatacaagaagcaaaagtgtgaggcgtttttttcttcttctatatcacaggtatactccaactaagataaaaattcatgttaaaatatttaacataattaaatatttagaatttaaactcaacttctaattaagttaaaactatttgatgccagttatataattagaatttaaattcaaaatttttaattaaatttaaataatttaatattaattgattcacatatttgattttaaagtaggaggatgtttgtaccaaacaaatatataagaattgtataagaaaaaagaacaaaaatggtaggagttaatgggcgttgggtcataaataggctcgaggacttcccagtcaatcaagcttaaataggatgatctcatgtcaaaatttaatatgatgagcgatgaccatacatttcttgctgcatagatgtatttattagataaaatacaactataatatttaaacataagatgctatgtgtgtgtatatatatatatgacgggatctatttgctaataaaattatggaatcttaattaggctatggcaaccaccggggtccaaggtaggggtgttttgagatttgggatatggcttaaagattagagttctaattttggaatgatctttccagcaatcagttatatacaaaactcacttaaaatggcttgtttatgaaatcatcacactaataattgagtttagtcaagattttgaccatcaaacttaacatgtctattatattacactacgtcgattttaacatttttagttgtcaatgaaagttctttgtcaattcaaactcaaatttgtttaaataatttacatgcatatcagaacagcagaataactcgcttcacttaaatttaactgattattataatgtataaatgacatgtcacaaccatatatattgctctattgacatacaggtgacctaaattttaattgggtgtcaaaacctctaatgcatactccttggcacctttaagtaacttagcaaagcttcataacagtactctgattaaattatatcatcggcagattttactaatttgggccttcattgttgatgtgtttcaaacttggatatgtttgcacaatgttaattacaataagacgatgttaggtagagcttttcctattagaaatatatatagatggtgattagggtaacatgttggaaaagaatccaaaaagtgtgatatctttgattcgttttgtcttattatagttgacattgtgcaactgtaacaagttttaggcttcacttgaatactagatgtgttttgctgaattattgttgaaactattttactctcactttcaaaaacataatttaacaggaatttatgttggtgaaacaaaaaaaaaacgaaataagtgaaacatctttattggctacttgataaatgcaa

SEQ ID NO: 24 - DNA, 2000 bp, Glycine max
  source 1..2000: mol_type = genomic DNA
gtattataattttattttttcattaaatctgtgagattatttttattatttaattataatattcaattatcaaattattatttttgaatattaatttattatgttactaaattataacgtaaacatatttagaaaccaaaataatcatctattcatataaaatatatgcatccttgaagtaatttttatattttagaattagtaattagtaagttaaatataaaaagtgtgaactaataatgtccttttccaacgaccattgcgatccgtttgaggtgacaatgacctttctctttattgcccatctctcttcgaatgttggattcaagtttatgatattatgttttttttttctaatttatttaaaaaaaattatcttatttatctcattgccaagttgcgctctctcgtcgtctcatttatttatcgcatttgtttggtagttatgcatcctcgtattaatttaattatttaataccgtctctattattgttctctctaacgaattctctctagggtgtgcaaattttgagacaaaaataattaaggatgtattcagcagagaaaaaagaaataaaaataaaaaaattaagatgaaaattttgaattaaaataaaacgtaaaagtgtaaattttgaataattttattgtttagttttccctctttttctctccaagcaaacacaacctaaaaaatcattacaatataatttaatatgttgttgagtatttataaacttaattaaatattttaattttattagattaattttgattaaaagtaaatttcgtgttattaaaacgtgctggaatgatttgttgatgctatcttagattctctttttttagcaaggatgttatcttagattctacttctttatttttccaaattctatttgattttttttcttaactttctattttattttatcttcagataacttgatcatgttattaatttctttacatattttttaagaattaatttagatattgtgtgtataagtaatacaaattgctcccccatatacaggagaaatcaccccttccattttaatatggtatcactatcacagctttgatattgcctttaaaatctcttttgtagatttcataaaggttcttgacatcgtatgttgcacagtttaaaagatttatcttgttgtttacctattatggttatattcatgaagatttacttatgtctatttggacattatttgcttttattcttaatagttaaacattgatgatgttagtttcttttgtatggtggttaagtctctcttttctgccttgatgatattgataacttcggcaaagtggttgaacatgaaaataccttttgcttatgtgattaagcctttttcgatctacttttaatttgttaagtggtcaagtatggagtgatatgggtgataattaagtttcttttttgttttttctttctaagttaaccttgtttcgttactccctcctttagaaacactatactaacttgattctatttcttcattgtcattttataatagaaaagatctctcaagtatcatcaaaacttgtttgttctaaagattaacttgatgacatgtttgctaagtttttacattttccctagataatatatgtattggatacatgtatatgttccagcttgagggtgtatgtcaaaatatgtagaaatattatcttgattctttctttgttttcttactattgagttttatttctttattttcttgttttcctattttattttagtttctttaaataactccatcatataatttagtatccttaaagagcttaatcataatcccaaattttaagcatttatttagattttgtgtatataaccatttgtacaaatcattccttttatctcaataaatatgaaatgattttaatataaaacttttaacttaatacaaaagctattttcaatttaatatatttatagaaaaaataaatttaattaaagttaaatatagtttcttcag

SEQ ID NO: 25 - DNA, 2000 bp, Glycine max
  source 1..2000: mol_type = genomic DNA
ggagacattttttaaatgttgtcaaaagtttttagcaataattttctaagtattaggtaaaaaatattactaaagatcatatacggtgatggtaactttacaaaaaatgataaaaggtaaagcatgaaaataccttacaaatctatataattttctccataaatataaaaataaattactttaccttttatgtttaaaaacactaatttgtatttaagaatgatattttaaaacaagtttgaaaattgattatactttaacttatttttaaaatatcattcttaaacacaaattaatatttttaacctcaatttttcataaataatttttcaccatcttttttaattcaattatattaaatcataaattaaatgaacctatataagttctattagaaattatagaattacatattttaataattttattttaaattttaatttaacaaaaaccaactttacaaaattaagattatcaaatttgcaaacaatctcggaaacacacaataattcaatggatatagtgaattcaagcaccattcggtactctttatgtttgggaagattcttggcttccttatctgaatgttgtcatttcacgaaatctccggccgttgccagtgaacacaaatttacaaacttttcaaattagagacactctcctccaggaatcatccacaggtgcttgttcaaaccatgacaaaggacccaatctttgctcttcctgtttcaaccatgaccttgacttagaccaaattgtgcacttccctttatgtttggaacacttatcttgccctttttagattttacttcaaatgctaaattttttatcttgggaatccgtgattaattaatctccttctgatttgatggtagatccttgattattatacttttgtttgtttccctgcaaagattttatgtgggttgcttttcgaatagtaagttggaggtcaaaataattaatcacgaaagaagtaagtttgcttgtcaggtgatagtcaattttgaaagaagtaaaagtttttatgtataaaaaatcattaagaaaaatattattttcgtgtttatagtttactttaataaatttctcgtagaaatttatcaaaattatttttttattaaatttgagtttctcaattaaatttatgaatattactttttataagaattatatatgtaaaaataaatgtttagtttaattgattatttaaatgtgatctaaatataaaatttaattacaaaatatatttgtaacataaggtgagataaataaatttgtaccatataactataatgatttaaagcatgaatgattaaaattagataatttaattatcaaacaactacttataaaattagatgacattatcttttattcttaacatttcataatttaatctacgggcatgattcactcttttcactctcgtaataaaatagccttctttgcttatatttatatatgaaagttaattctatttcatcattcaaataaattattttttaacttatttttaataaattatttttaattaaaaatatttttttcaacttcaagtttttacaatcaattttttcaacgtcattttttttttcaaaagccttcttttatcatgcaaacaaattatagctatattatcaaattcttgttaggaaagtggtatactcatctatacatcacagacttcaatacatgttaattacgtgtcgtacaaagccagcaccaaacccaatttcacaagtcacaagcgcaccctaccctttcctactccatgtccttaaccatataataaacaaattaaataacaaagtaataaaaaagtttattattacttttttttgctacgaaccatattgcaattaaatgaccatgagagtgtgtaaggagaccaatggttaagaaagtgcgacaactaacaaagccaaatcaaacatcaagtaaaagttacttcggttagctttgtcactgtccactgtgccctgaagaaagttccttaa

SEQ ID NO: 26 - DNA, 2000 bp, Glycine max
  source 1..2000: mol_type = genomic DNA
taggtaaaaacccaaatgatctatccgacaaactaatcgagcaaaagaaatgtgtatcagcctgtgccttctaacacaaccaaactgtttttaggtaaagagcaaaatgatttatctgaaaaaagaaaccaagcaaaataaatgtatattagcctgtgccttcaaacacctcctacgtccatgatattgcagcgacctaaaaaacaaacgtcggaagacgaacttgtagaaagatctgagcttttttctaaccaaaaatcaggtaaaaaaataagcatttgcttcaacaaaatcaaaaagcatatacaaataaacgccagactaaaaagaggcacacaggaaaatacaaattttggaaacaatgcaacataatcgaacacaacagtcacccacagaaacatcgaacctttttggttgcacaaagtcgtgagaagacgaacttgcagaaagatatggccttttttcgaacaaaggcataaacgacaaacaacagaatcaatgtctaaagataaacattagaaatatgaggttcaggaaataatagcaaccatccatccaaaaataaggcaaaaaaaataaacatgtgcttcaacaaaatcaaaaagcacaacaaaaatcgcacaaaggttgaacctttcttcttgttgtacaaaaagtaaaaaacaaaaaacaaaaatacaaaaagattgaacctttttggttgaacaaagtcataaatcgcacagagaagggaaaacatggccagaaaggtcaggagaacccaaaaataaccgaacgcaagaggtacccacatgaaaatacatacctggccaaaaaataatggtgacaaacacaaagctcacaaagccaaacacaaaggtcacaactttaatgatggataggaacacaaaggtcacagcgctacactgatggagaggaacgaaaacctctaaaccatggagaggaacgaaaatgaaaaaaggaacggtgagaagccgagaggaaaaaaaaggtcacaaccacggccatggaaaagaacgaagagctgtggagaggaacagaaagcagtgaagctacgaaaatgagaaactgaatggtgagtagccgaaacccttcggttacccaagcgcaccgggaaccaaaacgcgtgtacgcgtaccacgtatcccaattccataagaaattctaaaaacccacacgaaccattttgggcaggaacaaaatcccccagacgacgacacctcagaaagttaggggcattttggtactttctgcatgtgttagagccttgtttgtataaaggatatagatatttgtctaaggtgtatattgtaaacaaaatatgactttcctattcttacatggtcttttgtcttataaaagatgatagtgaccgagttgataattgttcacaataataatatgtgtaaagtgatagtcagttaacaagtgtcgactacccgtcgattccgtacaagatagtaatcatacgagtgtaaagtttcctatagagagagagacatgaatactaagggggattatttaattaaaatagaggattgttttaaataggtaaataataataataataataataataataataataataataataataataataataagcaaaggagaaagagatgttaaaaatggtggaaggaaaaagataaccctaaaaaggaacatttgaatatttggtggacgaagggatggaccagagttcaatactcgtgggtcgtggcgccgtcaacgtaaggatgcacgtaatgttctggttcatatacgacattgacactgtttaaaccatcagacacttgaaagcaaagcagtgagtgtgactgtgagagagcagcaatagagaatccaaaatcccgaaaatcaaaaacaaaacccaaaaagcaaaagagatatgattgtgtggctgttagttaccacgacaacaacatggtaactgacaagccaatcagggatcgacagcgcaacgttaagaaaactaccccggtttttcgtaaacc

SEQ ID NO: 27 - DNA, 2000 bp, Glycine max
  source 1..2000: mol_type = genomic DNA
ataattttaaggcatacaatttactaaatcatgccttttaagccattgaaccccaatctaattgaagaaactatatttaaaatttatattttctaaaaatatattaaattaaaaataacttttgtattaagttaaaagttttgtgttaaatttattagaacatttttcaaaataaatttattatgttaaaaaaatattaaaatcatttcatatttaggaatgatttgtacaaatctttaaaggagactaaattttatgaatatatgatggagttatttaaagaaactaaaataaaataggaaacaagaaaataaagaaataaaactcaatagaaataatcaagaaaacaaagaaagaatcaagataatacttcaacacatattgacacacacgctcaagctggaacatatatattacacgtatctaatacatatattatcttgggccaatgtaaaaacttagcaaacatgtcatcaagttaatcttagaacaaaaaagttttggtgatacttgagagatcttttccatgataaaatgacaatcaagaaatagaatcaaatcagtgatatagagattctaaatgtgggagtaacaaacaaggctaacttaggaaaaacaaaaaataaaaaagaaacttaatcaccgatcgataaagaaatcactccatacttgaccacttaacaaaagtagatggaaaaaggcttaatcagataagcaagaggtaatttcatgtttaaccactttacagaagttatcaatatcatcatggcagaaagagagatttaaccaccatataaggctgatgcaaaacaaattaatatcaacacttaaccaccaaaagcaaaaaaagctcgcatcatcaatgtttaactactaagaatgaaagcaaatcatgtccaaatagacacaagcaaatcttcaagaatataactataataggtgaacaccaagataaatcttttaaactgaagcaacatttaagatcttaaagatgccaagagcttcatatgaaaatgtacaaaagagattttaaaggcaatatcaatgctgtgacgccatattaaaataaaagggatggtttctcctgtatattgagcaatttgtattacttatatacacaaaatctaaattgattcttaacaaatatgtaaagaaattaataatatgatcaagttacctgaagaagctaaaataaaatagaaaattaagtaaaagaaatgaggagtagaatataagataacatcaaaaaattatttcagcatattttaagaacatcaaatttacctttcatcaaaattaatcttaaaagactaaaacatttaattaagtttataaatactcacacaaaatattaatttattttgtaattattattttttatatttttatttactattgcctcaaaatttgcactaaacaagagaccctagagatttcgttagaacaataatagacacggtattaaataattaaattaatacgaggatgcataactaccaaacaaatgcgataaataaatgagacgacgagagagcacaacgcgggaatgagataattaagaaaaaaaatctaataaattaggaaaaaaaagacataatatcataagcttgaatccaatgtacaaagagaggttggcaataaagagaaagagaaaagacgtccctgtcacctcaaatggattgcattactcattgaaaaggacattattacttccgactttttatattaacttactaattataaaatatataaaaaaatacttcaaagatgcatatattttattttattacataattacataacagaataatataaaataatgtaactacacattaaaacattaaaatagtgattggagtagtggtataagaggacgttgaattcacgcggaagagaaggatatatttcatgtttaatttgttgtcatgcctagttcaatgtaatctaataagtaaaaataaaatacaaacaaaataaagattttggtttcttaacaaaag

SEQ ID NO: 28 - DNA, 2100 bp, Glycine max
  source 1..2100: mol_type = genomic DNA
cgacaaacctatatctatgggtaacctgttgtcagtaagttatacttccagcggaatgaattccatgaaaaaaattccgttgaaaattccaatatttccaacggaatcaattccgtcgataataacttattttcttgtagtgtttttgttagaaaaattggtgaaatggtaactttaactcttgtatatatgtaaaatgttaatgacatgatgcagttattagcagagacttatctaagaaaaatagttttttataaaaaaaattactaacataaacctaaattgcaaataaccttatctaataatactactccagtgactcgactagtgcataaatgtcacgtattatggcatgttagttgataaaaattgtttttcacatattaattgattccattccattttggcggctattttgttttatgcttgctttcaatttgcattttaatatacaagggcagcaaataggatagggggtgtaggtaaattacgtagcttgtacttggaattattattttaatgaagcgaattgcaattctacatattactatattacggttaggttgcaaattttcttttataaacctcttgacccagtgtaaaagaattgactttaatgaacttaaaaagataaatgaattctagatcacacaaaatgatccaagtacacggaaaaaataagaaaaagacaaaatgggtgaaaattattcaaacttaattgccattgatagtaattctatcatatacaacttacattaattaagtatccaaaatacgtaatatggacgacaaatatcgttattttgtttatgttgttacttaacagacagtaagaattaaaatcaaactaatattttttaaaagacaaaaatcaaaatgactatattttttcaaggacccaaatcatatttaagctaattttaaaaacaaggttatcttaaccactagaataaagtattgttattgatggataaaaatattattgcactaataaaaatgtcttaataagtcactaaaacatcaacagaaaaaaattaatactgagtgatcaaattataattaagataattagtattaaaaagaaaaattattctttatatagttttacaatattatatttttttactaagtaaatatgtatgcagttctccacacattttttaggaaagttctccacacattttagttgtatgtaatatattttaatacatatatcaatttatttagtgttcgatcttatggttggtgttaaagcataggataatagattcaaatttcacaatcaaccttttttcaattttcaaactatttttaagcaccacttaaaactttcacggtaaattaagagttagacaaaattttattaagtttaaaagtacgagttctagattttaataactgaaactagagggattaaaattctgagtcaaaaacagaattaagccaaaatattaattcttttagcttaagttgtttttttatactccttctcttttagaatatacaatgtttaagatttttttataagtattaagaaatgtagttaatttattgaatttcaaagaaaacattaagtaattttttaatatgtcattttatctctaaataatgattcatgtattcttcttatctaatactctcattaggtatttattaaggatatccttaaaaaaaaagtatctaatgtagcattgatggtgtaaaataacatatattctggtatggagtgagtacctatgcaatgaatttattttcttttaataacaattacttagctaaatttaaaatgttaaatcacattagaccttttatttgaatttcaaaagaaaattaattaaaaaatattgtaataatcttggaacaaaattaagtttttatgagtattagaattaaggagcgattgattgttgaaatttgttaataactataagttataactattaaacgaaatgagcaaatcttgacatgattttaaggcccaaatacaattgaagcccaatgtttttaaaagaatattaaaaaaaggtgaaattgaatctacgcttatcccgccaatgggaaatttgaaattcaaaacgaaccgtttgcgtttcatttttc

SEQ ID NO: 29 - DNA, 2200 bp, Glycine max
  source 1..2200: mol_type = genomic DNA
acattagcgataacatgaaattagaagtgaaaatatgccaggctactccgtaggatcttcgttaagcgtagactttttaatttattaaataaactagactgagactttttaaaaggcctatttgattaaaaaaaattagattttagatcatttaaaaatcttttttttaaggaaagcatctcatattatttcaactttaagaacatacttaatatgtacattgtggaactgaattaaaaacttgtgaactagcaaacaatctagatgtcttaactagtgtatagtgactcacttagccatgtctatggacaaacttaacactaaagttttgaataaattaaatgttcaatctacgtacaatttttcagcaaagccccaaattcattattcttgaagctgaacgagttgaagttttctaccaccaatttgaaccagattcactatttaagaagtcttttaaactcaacagtctggtcaatttataattaagtaaatataaataataatcttttgttaatattattattatattaactttctataatatgttaattatatgttaatttatctttttaaatattaagcatttgagctaaatatagaatcaagcctataatcctattgaaagaaaattttttatatgccctatctagaggcgttaatgcaaaataaacttctaatatcatgaatgccatattagacattaaatttaagcttatatcacataaataaattaaggttgattcttatcagatttaatcaacatattttctctcatattccatggcttgaatagtagtggttttagttttcataatttatattccctttggttgttttcataatttatattcctattactccaacttacaggatttacaccttcaatgtaacgaccaagaaagaagtcggctaagaaaatggcatgcataaccaataagaataaaacaatagatatattaatgatgagagaattaattaaaataatttaaactaataaaatattttaaaaaataataaaattatatctcattaatgttatagataatttttaaaaaaacataaatttaggataaatttaatattattaactaaattaaccaaatttttttatttgtctcgttgaatttggatcttataaaaagaactggagtacttttacataggattttttccaatggcctattaatcggttaatttatttagatataattatacatatctaaaagctttaatataaaactaagtaggttaataagctaagtcaagttttcaaaaagctcaaatcacgcttaattaaaagtatttgacaagtattaggctaacttgaatgttgattttactaaataaatcaaactcttttttaaaaaaataaacttcaattaaataattgagaatattaaaaaaaaaactttattcgacccaatctatttgcactcttacttttgcaattaccacctagacatggttagacaaattaacttggagaggccggtcaaactaatttaacttggagactcgttgttgaattagagatctgtgatttggcagctcatattagtccaaatgaattggtgttcggcaggttggatgaatgggtgtgttgtaattttttggaatttttaaagatatatagtacgaatcttacaaacttttaagtgtaaaatttacatgttatacattttttattttttttaaaatttatttcaatttgtacaatgtaatcttgaaatgacctaatgctttaagtgagtattaatcatcgattcgaaaatgataatattagctagataaactatttgtaatttttcatggccacaacgagataagatgtgggtttcatgtcccattcatgctaaaaattcaatgttctttttattacaaatgaacaatacaattatcaatttaacaaataaaaatactttcttaaaaacataacttaattttaaatagttttattctaaaatattcagaataactcaatttctgaatctcaacgaatatttgtttcttactatgcatctagttgtatattagaagctaatgagttgacgcagtgatgcaagatattgttgcatcacatttaagaaaataatctatttaatgaaacaatgtgtgtctcattctctccgtatataatttttcttaaattaagagcagtcatatattgcaaacaaaattaatctctaattatgtgagt

SEQ ID NO: 30 - DNA, 2000 bp, Glycine max
  source 1..2000: mol_type = genomic DNA
ggaaaatgaatatcgttttgagtgtttcttatcacctaattcaattgtctcgcaaaataaaagtaactaattaaggcaaaaaaaaatccccctttgccatttgaatatataaaagattaaagttccgaagaataataatttaaatataatgtaattcactttaaaagaatattatataatagcaaaaaaatgttaaacattaaaagcatcttggtcaatatgattttttgtctaatgaaaaaataaattatatataatatcactgtatgtttatactaataatttatactagttgtttgtagtcaaaatttactatttgggaaaaagtctgaactgtcaccatggaaaactgcaagtttccagatagtggaattacgtgttgtcaaaataacttgacaaaatgtttgattaacaatttgcatatcaaaccggtaaaaacaaaggtgtaataaagaagcaactaaacgtacataaactaatcaattggaaataaagtcgatgaccaacagaaaactttcactaagtaatactaatagattgaccaaaataaacagatttggtacaagaaaagttttctgtaaaaaaaaaacacaagaaaagcatgaaacgtatatagatacaaaatataaatctaacacgaaagattaaattaataaacgacatagcaagcaacatatataaataaacggaagatatagaagaaaatgaataacaaaactacaacaggcacgatgcaagtgaatgagtaagaaactttgatttacgatctacacaaaacttgaagtcaagaagccaaagaaatcatagaaagaatcaaaaccttcaaagcatgaaatttgaaaataacctgcgttaaattctaaaaagaaattacaaaggaaattgaaggggatggcagaatttgaatggaatcttgttctttgttcttttgtaggattgattttttttttttactccttattttctattttgctatatttcttaacagggtgaacttagacagctataatcgactttttcttttcaaaaatttgaaaaaattcatacgagcattatcagaaagtgtatatataatatcttaaaattaataaaataacataatttaactatattgaagaaaattatatattgcccatttaaacattactaattgtgcacaaaattgattttagttcataatctgactggatcttcgtttgacgtaaaccccaatttggattcttttcatttgtttgggaataaaacaatgactggtaacgggagatgatgtcgttatcattgcccatgttatgccctcggtttaaaggagtgaaaataaagcaatatcccttccagattctttttggttttcctttcttggaatttgtctgctcttctattatcggtgccatttttatcaaatctcctggtcctgtgtttcccacactatacttaactccttactgcggtacgtacagttccttcttcccacccttatatttgtaaattgtagggtttgaggttgagtaaaaattgggttttttatttatattttgtgttagatatgatagaacggtgaatttaaaagagagtgaagttggcaaaataagccgacttgaatatacttatatacccattaaaatgtaggctttagactattaaatttaaggttttaataaattggagttatgtttgttagcctaatctaatcaagctcgtattgcaagtgtaactgtgattaatcatttaaactcatatcataatttttttaattaagaaagtttaactctattgattgaacttgatatatgagtttttcttaagacaaattatatatatatatatatatatatatatatatatatatatatatagtgatatagagtaaaatcaatagattgttgtattatgtgaattgttataaatttctagtatctgttttttatttctaagaataatagtcctaagattgtttttttgtaaacaaatacttgtttaaagctggaataatgacaagtgtc

SEQ ID NO: 31 - DNA, 422 bp, Dahlia mosaic virus
  source 1..422: mol_type = genomic DNA
atcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatcatcgaaagttggagcaataaactctctcttcaacaaatctatcttttatcttttatc

SEQ ID NO: 32 - DNA, 3681 bp, synthetic construct
  misc_feature 1..3681: note = Synthetic sequence. Codon optimized LbCas12a
  source 1..3681: mol_type = other DNA
tcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 33 - DNA, 36 bp, synthetic construct
  misc_feature 1..36: note = Synthetic sequence. Nuclear localization signal from Solanum lycopersicum preceded by a 6bp sequence coding for methionine and alanine
  source 1..36: mol_type = other DNA
atggcgggatctaagaagagaagaattaaacaagat

SEQ ID NO: 34 - DNA, 33 bp, synthetic construct
  misc_feature 1..33: note = Synthetic sequence. Nuclear localization signal from Solanum lycopersicum fused to a termination codon.
  source 1..33: mol_type = other DNA
ggatctaagaagagaagaattaaacaagattga

SEQ ID NO: 35 - DNA, 2008 bp, Zea mays
  source 1..2008: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 36 - DNA, 3681 bp, synthetic construct
  misc_feature 1..3681: note = Synthetic sequence. Codon optimized LbCas12a.
  source 1..3681: mol_type = other DNA
tccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcac

SEQ ID NO: 37 - DNA, 500 bp, Medicago truncatula
  source 1..500: mol_type = genomic DNA
aagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctc

SEQ ID NO: 38 - DNA, 864 bp, Glycine max
  source 1..864: mol_type = genomic DNA
caaagatgagaaatccatttatatgttgaaagaaaggggaaatgtgccttcacttccatttcacaaatacacaaaaaaaaaaaaaaatgctagtagtgtaaacactcaaaacactcaaattaagccctttcaacctttctttcttatgagtttacaatctcaaagcccatcaaagttcacgaccctattcatctcttccaacatgagcaacccttcagatgaaagggagcagtgtcaaaagaagacgaaatccaccatatgcgaagcctctaactttaggacatcaaggagaagattctgcagcaacaacaaaaatgaagaggagatgaacaataagggagtttcaacaacactgaagctttacgatgatccttggaagatcaagaagacgctaaccgatagcgatttgggaatcctaagtagactcttgctggctgcagatttggtgaagaaacaaattttgcctatgttgggtgcatatcatgcaagagctgcagaaactgaagggaccccagttagagtttgggacatggacaccaaatccatgcaccaactcgttctaaagcgatggtcttcatccaagagctatgttcttattggaaagtggaaccaagatttcgtcagaagaagagatctcaggaaaggtgatgagatcggatttcattgggatccatataattgcgttttcaatttctgtgtccttaaacaagctatgccagagaattaatttaattttaagtgttagctttattattttactttcaaatcattgaggaaaacaatggcctatatattattcctatatgtaacatacaataatgttattgcaatagcgtgtacttcaacctaattatttaataccaa

SEQ ID NO: 39 - DNA, 27 bp, Glycine max
  source 1..27: mol_type = genomic DNA
tttaggacatcaaggagaagattctgc

SEQ ID NO: 40 - DNA, 27 bp, Glycine max
  source 1..27: mol_type = genomic DNA
tttcaacaacactgaagctttacgatg

SEQ ID NO: 41 - DNA, 27 bp, Glycine max
  source 1..27: mol_type = genomic DNA
tttgggaatcctaagtagactcttgct

SEQ ID NO: 42 - DNA, 27 bp, Glycine max
  source 1..27: mol_type = genomic DNA
tttgggacatggacaccaaatccatgc

SEQ ID NO: 43 - DNA, 400 bp, Banana streak VN virus
  source 1..400: mol_type = genomic DNA
agacatcctggaccaatatgctgaagattatgctacctacaccaggataggacttgaagcacttaaccttgaagattggttcgaagaaccagaacccgatccacctaaccctgtggaccgccagaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagca

SEQ ID NO: 44 - DNA, 1594 bp, Medicago truncatula
  source 1..1594: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacgtactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagagctgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagggt

SEQ ID NO: 45 - DNA, 2378 bp, Glycine max
  source 1..2378: mol_type = genomic DNA
acttgaccaaacggagttcataaatgtgctggaagattcattatctacgaatattaaaggatatccatccaatccaaccaatttattattttttttatgtttataactagatggattgggtgattagtaaatgcatggattgaatagtcactcctataagtattctaagattactttagaaaatggcatatttaatattggcctgtcattacagcataaatggatgcaggtatatatataatgcggtgacctgtacaaacaaatgttcacatcagcacatacacttcaaaatcaataggaaaaactgtaaagatgcttacacaaaaatatcgaagatgtttacatatttcattacaatttgtttattttttctttcgtgtgtgtgaacaaaataaatgatctgcacatgtatcatggttatagtaacaagtcccagctcccaagggtagtagctgcttaaagacacacggaaaccctcgaggataatggtggagagagagtgttgaaatttcaaccatctcacttttttcttctaccttattctttagccgaaaattaagttctaactttaaaatacagatttgcttcagaagttgattttttaggtttttttttttctggaagaccaatattaatttgtgatagttagttaaaaaaattatattatgttttgtgatagttagttgaaaaaattatattgtgttttgctactaatttactttcaaaatataaatatccaaatatatatataaatcttatcacttgaaaatagatggtgggtatatggactcaaatctatggatcggtctgtttaacccacgaattttgcagataacaagcctttttttaaaagaaaattcgcataactatatccaagtttttcttagttcggtccattaagctagcacatctagtagtctttatccatggacatattattaatttgttatgttaaaattttgatttttaatctagtggtatattttagtatgatagattttaacttaaaaaatgaagtcaattttttattactaattttatacatttttcacttttaaaaaatatttatttcataagaaatatttttaaattactatttgggtccatggatcaatttatttatctgagtttttatgggctagatacaaatcttggaaaaaagccaatttatttcatggattggcttatttgatccgtctaaaatgcaagcttcacaagacaaaccttaaacataccataccatccttttatctatctctacttcaaaatcaatttttgcaacattcactcaatggtgcacttaattaacaaaccctcatccatatacacatatttagagatcaacaatatgattatctatcaaaaatacacaaaatcagtgtgtgtttgggtaggcgttgacaaaaattagttttgaatgaaattgattttataattttttttttgttaaaattgattttgaagtaatattatttatgtttggatgttttattaaaaaattaagttatgaataaaatgaagtacataattttggaccaaaaattattcaaaattatttcaacccaaaattaattctgtatccaaagtcaattttaaatttttctttgatgtgaaactaaacatgtaaaaatgtatttaaattaaaattaattttagacttataattaattctctgtggtcaatccaaatacacactcacacttagaatgtatgaaatttcaatttttaactttctcatctatgagctgttcctattcctccttcccctcctatgccctcactagggagccagccagccatattccaaaagcccttattatcacacatgggtccctccatagtcaaaataaaaataatatcatgatcactgtttggccataaagagctatacgacacacatggacacagtagtacactgcccaaccaatcacacgtcgacagcacaccgttcccaaactacttcacctttcccaaaccagaaaccaaaaccacttgtcatcaaacccctgcccagatagtttttctccatttcaatattttacttcaccttttgaggctttgtgggtactacaaaacataacacaattgaactcactgtgctttcccatgacacacatctatacttgtccagaagaagaaagatccatgaactaacattcccacacgctcgcttcaccatatttgccacccttttaaccctcacttcttgggtttattttgctccttttttttttccttgtttggggtttgcatttttcctggttgaaaaagggaagaacttgaactaattggttaagttacctatctatctatctatctagggtataatattttatatcagttattaaaggaaagaaagaaagaaacgaagccgttaa

SEQ ID NO: 46 - DNA, 1629 bp, Glycine max
  source 1..1629: mol_type = genomic DNA
ttttaaaaaataatttctattcttaatttgacaaacaaggttacatatcaatagtactggcttaacaattgttttgtggtaaaatattcaatttgcgcattattttttacttatatcattcatttttccttattaaaagcaaaatattgtacaagattaaccgttaaaaagtctagggaatgaaatcaatttttgacgaattaattaaagtgacagatagaattataatatgcatgtaaataactaaatgtttttttaagcacctacgtattcatgtacttaaatactataacgagtacacatacacatatgtatcccagggaagctgcaatcgctgtcagagatgaatgagcttttttcgggattgaaatgaagtaatgtgggcgcattaaaaaagaacaccaagtttttagtcaatttgtactactttcagcttttcagaaaatgaaaaaatgtaattttatttttcctgacgagtttgtatatttgcggtttagtttttacagaacaaacagctgagaagctattacagcttctcacctaccttttacatttcctgtacggctcaacttcaaaagttcattacctttcggataaaacccatcaaaactagatccacaaagttagggctgttttctttttcaattgtataactacccttaaaaagttcaattaataaaccttctaaattaaaagctaaatcaaactatcccttagtaagcacttgtttttttcccaatttttttccatatgtaccactcttctctttctaacttcctttggcattctttttatgtaatttaattcgtctttaccataattaataaattcgccgtgaagtatatgtacttatgttgatgtatgtcacttaatttttgtataacattcttctcgaagttgatggccttgaatatgctagtaatattttgaatatggtacaaaccaaaaaattatttgtgtccttttgatcggtgtctgtcctgatacttgagctagattttctgcctctgccctaacccagattggtaatattgtagttgtattcacaaaataaatgaaattatgatctaagtacttcattggcctaaactaaatctccctttctgttgtcctcactttcctcaacaacatacatacttctctttttctttttttaaaaatatacttaaaaaaaacaaaaaataataataacaataataattctttaatggtacttagtacttagcagctagtagtctcctttcaaagatagagatggggaatctcagaaaatgattatataagagtagtaggactgtacttggaccgttaagtaggagcagagaagctacctctgtgttataattgtgatctttttactcactgcataatagctagggttttcaccacactgaaatttggtttccaatctgcagatgttctcttccacatatgactccaactttttccctaacttccctttgtctacttacccaatccttccttttctcattgaatctgaaaatcattttgcaagccacactcttctggttgaggaccctcttgttgttcccctcacacatcatcctcctccattccctgaagaaaccgttgccaattttgcagttgctgatcatcactgcactgccc

SEQ ID NO: 47 - DNA, 2374 bp, Glycine max
  source 1..2374: mol_type = genomic DNA
aaaaaatataagcagtttgtacatacatactaaaaaaataaaaataaaaatcttattttatgaatataaatataattaaaatttaatattatattatttatgctgttcaaaaataaaaagtgattttgagatataatatacatacgacaattaatacaatattatttttgtgtctatccctctcttagaccctattatttatcacatttgaaattcagaattttctttttctctcgttgttgtacaaatatctaatcgttaattattattaatgcattaaatatttaaaatataattaatgatattaattttcctagcgtaagactaataataaatatcgacattatgcgagattatgacatgatgttatgttaatagtacagaataaagtaaaaaaatatatataacaataataatttaaaatatttaaaatgacatataaagtagttttctaaaacttcagtgattagttcaatttattatttcgcaacaattatgatggttaattaaggtgaattagaagaagaactgaaaattaagataattaaggagaacaaatgagcacttcaaaatttgctattaagccgtctctgctagccatgcatgttggatgtaacttcacccgattgatgggtccttaattataatttttcttatgataaaaaataaattgctactaaggcgtactgaactattcgaaatgacaacactacaaggcctcgacaacgttaacttttttccaatttagtggggcgttacaagctaaagcttttttatttttgaactttaaattgctctttcactttcatttaataacgcctaaacctagctacgagcaatataacctgggtgtatatagctatatatatcatatgcagagcaatctgtaaccccaaggataaaacgttgtgttgcttattatagaaatcacagttttaaatggaacgagtgacagagtttgtttgtttctaaataggataggaggcatttaatcggaaattggacaggagaaagttgttttctttctgcaaattatgcctccagaacttgttttacggtaagaaaatttacaccaaatttacaagtattggttctgaacgctacttgcggaaattttatgccgccagtgacttcttggcactttaaataagttaattagttaatcatttgaaaatatattaacaagaagaatttttttctagtgtaatcaagaggtattttaagcttctatcttacactcaattaatcttctattctcctattgattggtctattttaaagtaaagtaatatattcaattacgacttcatgctaaattcaaagattataacagtaaaaacaatttttattcacctctattcaaactgccagtttagcgcaagtgtttatttttctgataaacttgcggccgcacaaaacccagcattgcttcaatgtgcataagcatgggtacggtggtggtgtttctccaacctccatttcagattgtcttgatttgtaattcaaacacacacttagtcttaaaatcatattttctcatatctcaattgtgatttttccaaatgcgttgtgtgtaggcgaagctaagtttaggttattgctcaagtgccacaagtttgagagggttttactgtcttttttatttaatctagaaatatgatttttggacacgtctacttgttacattccgtttacttatggtagtatttgactaataaagacattaagattatagtagcatttgactaataaagacacgcgtacgtaggtcactattttattggctaagtaaaaaaaaaaaaaaacacgaaaaaactttcaaacaaaggggaagcggagaaatcagcctaacttgatattagctgtacaaaatacataaatagctgtcggaaataattatctcttgcagtatatatatagaaagtagcagactctaaaatataacatgaagatgtgattaagcatttattccattatatggcccataaacatgcatgatgcaaatagaaggatgtagggtaaagttgccctaagaaacgaattgaactgcggtcgttgcggtatgagaaagagagagagaaaagtgagtgaaggaaggtgggagaatggtttccgtgacggagtaggacggaaagtaagggtccccacagtgcacatgggagcggatttgacgatgagacgagaaagagcacaagacgtttatgacaaagcctgcaatagctttcgttaatttggcaaaagacccctaccatttgcggcaatgcacctccccgttctattatttctctttccctcattcattatatcccctattctctgccccagatcgatcgaacaagtgaattagctagaagcattgaaggacgtagctgct

SEQ ID NO: 48 - DNA, 2711 bp, Glycine max
  source 1..2711: mol_type = genomic DNA
atatgatggagttatttaaagaaactaaaataaaataggaaacaagaaaataaagaaataaaactcaatagaaataatcaagaaaacaaagaaagaatcaagataatacttcaacacatattgacacacacgctcaagctggaacatatatattacacgtatctaatacatatattatcttgggccaatgtaaaaacttagcaaacatgtcatcaagttaatcttagaacaaaaaagttttggtgatacttgagagatcttttccatgataaaatgacaatcaagaaatagaatcaaatcagtgatatagagattctaaatgtgggagtaacaaacaaggctaacttaggaaaaacaaaaaataaaaaagaaacttaatcaccgatcgataaagaaatcactccatacttgaccacttaacaaaagtagatggaaaaaggcttaatcagataagcaagaggtaatttcatgtttaaccactttacagaagttatcaatatcatcatggcagaaagagagatttaaccaccatataaggctgatgcaaaacaaattaatatcaacacttaaccaccaaaagcaaaaaaagctcgcatcatcaatgtttaactactaagaatgaaagcaaatcatgtccaaatagacacaagcaaatcttcaagaatataactataataggtgaacaccaagataaatcttttaaactgaagcaacatttaagatcttaaagatgccaagagcttcatatgaaaatgtacaaaagagattttaaaggcaatatcaatgctgtgacgccatattaaaataaaagggatggtttctcctgtatattgagcaatttgtattacttatatacacaaaatctaaattgattcttaacaaatatgtaaagaaattaataatatgatcaagttacctgaagaagctaaaataaaatagaaaattaagtaaaagaaatgaggagtagaatataagataacatcaaaaaattatttcagcatattttaagaacatcaaatttacctttcatcaaaattaatcttaaaagactaaaacatttaattaagtttataaatactcacacaaaatattaatttattttgtaattattattttttatatttttatttactattgcctcaaaatttgcactaaacaagagaccctagagatttcgttagaacaataatagacacggtattaaataattaaattaatacgaggatgcataactaccaaacaaatgcgataaataaatgagacgacgagagagcacaacgcgggaatgagataattaagaaaaaaaatctaataaattaggaaaaaaaagacataatatcataagcttgaatccaatgtacaaagagaggttggcaataaagagaaagagaaaagacgtccctgtcacctcaaatggattgcattactcattgaaaaggacattattacttccgactttttatattaacttactaattataaaatatataaaaaaatacttcaaagatgcatatattttattttattacataattacataacagaataatataaaataatgtaactacacattaaaacattaaaatagtgattggagtagtggtataagaggacgttgaattcacgcggaagagaaggatatatttcatgtttaatttgttgtcatgcctagttcaatgtaatctaataagtaaaaataaaatacaaacaaaataaagattttggtttcttaacaaaagtacttttactttaaatatatatttttatctggtttttaaacatgcacatatttaacataaaagttcatattaaactttttcctacatacttggatcaaatagtcacgtattgcaggtaaaaaataatagtgtagcttatagaaatcgtagaaataagtctataaaccagaagaaaaaaaacattaaaataatagtatagaaatctatatcagtgtccccagttcttacattcatgacccatttccccataaactctttgcagataatgcaatggcaaaaccacacagaaagtgacccctgggaatcaaaagttaaaaccaatggcacagcatagcacagtgtacagtgtttatttactatatagcaaaacactcactggcataacactttagggagagagagagtgaaaacaagtgtaaaaagagagaaagttaggagggggatagagagtgtgtgtgtgtgcagagtttgcaggcttgtagcagaaatggtggcagatggttttaactttatgtgtgaaataattttcttctatctcttttctctttagtgttttctctctctctctctctcttctttttcttcctgcatcttcttgtgtttagggagtgtgatgttttgtggcagaagaacgatgtgattggacacagccaaagctgtggacttgttctgttactactttgtaattgtaatcacataaaaggctagagggtatgaagagtgcacagaaaaatactagtactagtttcaaacaaaactcaccttactactacccttccatctcaagccatagttgagttgagtggtgcacagtgtcactatacataccactaacacccttttttggttcttgttctgtggctccttgtgctttgagcaagagctttttgagaaagagcttggtggtggtggttgttgttgagtggtttcatggttaggctgttgttaagttgaagttcatcagttgcagct

SEQ ID NO: 49 - DNA, 4106 bp, Synthetic construct
  source 1..4106: mol_type = other DNA
acaccaataaaaatacacagcaataaaatcgctacgtatatatatatataatatgtattatctattacaagatagtaatagagtatagcaagttgtatcatctaacaaactatgcgaataaaatttgaacattgtgacatgtagatgtagtgtaatttagctaagtgcttatcatcagtaacatagaccgacttaactttttacgaaaaaaaaaaagtaacatagaccgaaaaaatgcatatcgtaaatttaatggaaaacacaatttacgataagtaaaaaacaaaaagaaattacgataagtcgagaaaaatgcaacaaattgagataaagtattgataaaaccatgaaagtgtcggcgtatgtaaatgcggtgattaatgtgatcattagagcgtgtgtgttaaacgcggcggttttagtggagattgatcagctgataacactcttaccgggacgaatctaattccatattcatggcttgttaaaacctaagacatacgcaatctctaatttgctagtatagttagttctatattatttttcgactaataatgtaaacatatgattattaagtcgcaaaaagagtgcttaacaaccaaaaagtggattaattaacttggtgggaaaagttacaaaacctttaatgattactctttgtaccaagaatagtggcgaagcactataagagcagagaaaagaagctcaataatgtactaaaagttgtagatttttacagcttaaatacaccaaaattaatagaaaagttggtaattttttaattcatggctactgatttagattttagaaaacaatagtagtatcattgtcacatcttaaacacacaataggtatgttttaaatcaaaggccgtagttaatttgtcaaaaatgtatgcatttggtatttggatgtctccgaaaggatggatatatggacttgttagataatttcatacctcagtatcaatagtcatggagcccaaattgctcaaaaacatatttttaattccaagactttgatgaagacgtaataatgagtccaatgggccatcagatacaatgttcggaatttaacgggtttgttagttataagtattgggcttgacctatctggttcaatgatatgtaggaacaacccaatttgcaaagctttattaaaagactctttagttgtcgtcaaggtttaacttgtagtagttggtaagaaattctacgtgaaataggcaacattacaaaaacaaaaatcaattcgaaatcatacaaaacgaaaccaagtagtaaccaactacactattatgacattaatgattagacattcccaaatcatacaagttcctgtcatgaaggaaacaatggtccgtatttgcaaacgattacaaaaattcaaaccaaaaatgaaaaaacgagttaaattatttggtttataaaaatagtaatgtcaacagaagactagattgggaaacctgaagcgaacagagcttttaaaaacgagtttgaacggctgggatcatttggtacaatacccaccgtaagtttgtttaccctagggatgcaagccaaaggcccaaatcagttactacttactgctacaaccatcgtctcagctttttgtctcagctttttactaatgaagcatacaatttcttgggcatgtcacatctcgacacgtgtccactattctcttctcttattggctactcgttcgtaggcttctgttaatagatgatctctctataactctaacagtcttttctttctctttatttcgttttggtattttaagtttcaaattgaaaataataggaggaaaagtctagttttaaatattgtttttttacaagtgaacgtgaaccaatttacctctttttttttatatatcctatcggctaatctggttagtatcggtagaaatgcaccgaggtgctacagagattaatgctagggatagtcagaccgcttgtatttctgactatcaagtaaatctacgcccaactcacatatttcccaaacaaatgtgattttttttttctttttttttttctttttttttttgtaacaaatgtgattttgttttcaaggaaaatagaacttacgtttgggaatttcacccttcactaaagcttccttctgccattagaccacaaaggcttgggcaatttaccatttttgtaaaagtagaaaacaaaatgcctaaaatgttcatacttcattacatcaacaaggttatgcccacgatatagaggcatgtaacatttatatatatagtggaagaagcctacgagctttattaataagtataaactctgattattaggtaaataaattacttaaaacgattactcaactgacaaaaccgtagttgaataataaggttactatgaataccgattgaatattgcaaagccggaattgaaaaatatataacagatcaaatgttcaagtgtggtcataattctcacataggtcatatagctgaacccatgcatctatttactagtctatagaaagtactagagacgcatacagctgaacctactctattcttttattaattttggttctcgtggatacaaaattcctccaacatttattagaacgaataaaaccaatatgatgatgattagttattggtaaacatataaacgttgagtaaacttcaaaatagattgaagtactattaagacttgcattttttccccttgggttatattcttgaatcgtttcgaagtattttaactttcaagaatagaaggttcctcaactataaacaattacattaatcaaaaccatttctatgtaaacaacataatttttgtatattttagtcttccccaaaagtttgaccgatagggcggtttagaccgtatagtacgactgtacaacaaaaaggactctggagacctaaagatccaaaactatgcaaaataaagatacggtcggaccaatttaatctaacaaaaccaaatccttatactaaactatttaccgatacatttccatataacacagtacacacaattaaatcaaacattattggaagaacaagatagaatattggcttaatctcgaacgattagagttatcctagagcctcggagcttttgtcacatataatataaactatggtatatataaacatgactctcatttgtatttatcgcaaggtacaattccaccaatttttttcgtcccactcatacagctttaattgtgaaatcaatccataaaaaaccaacatgtgacatggtctctataactataactataagatagtaaaaaattcacatcaacataaaagaaaaccaatcatattggctaaaaaaaactaacggtcgaaaaacgtataaccacaaaaccaaaccggtccaaccggtgtccccaatcactatcaaagcattaactaactttcacaaggaaaagcatagttcagtttctctacatcgcttcccatcctcttaaccctgtttactcgaatcatccaccgttggatcaaacacgcgctacaaatctagcgcgtgaccgaggtttttacacagtggaatattaccatgcattggaaagcggcgtctacaacaaacggcgggtcatgtcaccgtcaaaatcaacctttcttaattcctaacgccgttacttatctccgtttactaaaaatgttaatgcgtgtgagagtgaagatcatatactaattagaagtggctaatgttttaacgtgacattattatcatagttaatggttcgatcagagttttaagtagtaaatgatataagtgtgtgtatataattgcatacatatatactctcacactctgacagatttgtcgtggtcttagtattctctttcatggctagttatatagggctctagtacattatctctctctccccatttctctgtctctctcttctttaacatctctggctctgcatttcttgcagttcacgctaatcacctgttttggtacccttttctctctgcaacaatagcagaacaagcctgaagtttcgttcttttgtgttttggtttcctctgctttgttgttattgaatcaaaacttcaacgagcttcttggcttattaccccaaaaggaaagaagaataagaaga

SEQ ID NO: 50 - DNA, 500 bp, Glycine max
  source 1..500: mol_type = genomic DNA
ttaatcatctgaaactgttcaccatgcatgcaatcttgtgaaatatatggttttaattagacttcaatcttatgttggctattgtactaataaaagcatgtcatgttattttcatttgattttatctgtactttggtttgtttgaagaataaagatgagcttgctatgcatgcatgcatgccatcgattatcagggtttccttttttcttttctggcttcccatcaatttggtgtgaattagtgtgtgtgatatattatattatgctatttatgaaataaattgttggttatatttgatctacaatctacatacatgtgatttttatcaacaaaatatctcgggaaacaatacctttttggtagcaaaattcaaataatactattttaaataaatcaaagttaaccaataccttattcaagttggaggggtctcaaacaagcaaaagaattcaagttgttaatgaacttcggttaatgataaaagaattcgcatttaaaa

SEQ ID NO: 51 - DNA, 1542 bp, Glycine max
  source 1..1542: mol_type = other DNA
atgtctccattgattgttgccttggctacaatcgcagcagcaatcctcatatatcgtataataaagttcataacacgcccatccctccccctcccaccgggccctaaaccatggcccatagtgggaaacttgccacacatgggcccggtgccgcaccactccctcgcggcgttggcccgcatccacggcccgttgatgcacctccgcctcggcttcgtcgatgtcgtggttgccgcgtcggcctcggtggcggaacagttcttgaagattcatgactctaatttcagcagccggccaccgaacgccggcgcgaaatatatagcttataactaccaagatctggtgtttgccccctacggcccacgctggcgattgctccggaaactcacctccgttcacctcttctccggcaaggccatgaatgaatttagacacttgcgtcaggaagaggtagctagattgacatgcaacttggcaagttcagacacaaaagcagtgaatttgggacaattattgaatgtatgcaccaccaacgcattagcgagggcaatgattggacgaagagtgtttaacgatggcaatggagggtgtgatcctagggctgatgagtttaaggctatggtgatggaggtgatggtgttggctggagttttcaacattggtgactttattccttccttggagtggctagaccttcaaggggtgcaagctaagatgaaaaaattacacaaaagatttgatgcatttttaaccagcattattgaggagcacaataacagttcttccaagaatgagaatcataagaatttcttgagtatcttattgtcacttaaagatgtgagggatgatcatggaaaccatctcactgataccgagattaaagcactacttctgaacatgtttacggcaggaactgacacatcatcaagcaccacagaatgggccattgcggaactaataaaaaacccacaaattttggccaaactccaacaagagttggacaccgttgtgggtcgagataggagtgtcaaagaagaggacttggcccacctcccatacttacaagctgttatcaaggaaaccttcagactccacccatcaacccctctttctgtgccacgtgctgctgccgagagttgtgagatatttggctaccacatccccaagggtgctacactcttggtgaacatttgggccatagcccgagacccaaaagagtggaatgacccattggagtttaggcccgaaaggtttcttctaggtggtgagaaggccgatgttgatgttaggggcaatgactttgaggtgataccttttggtgctggacgaagaatttgtgctggacttagtcttgggcttcaaatggttcaacttctcactgcagccttggcccattcatttgattgggaactagaagattgcatgaacccagaaaagttgaacatggatgaagcgtatgggctaaccctacaacgggccgtgcctctttcagtgcaccctaggcccaggctcgcaccacatgtgtactcaatgtcttcctaa

SEQ ID NO: 52 - DNA, 27 bp, Glycine max
  source 1..27: mol_type = genomic DNA
tttccttgataacagcttgtaagtatg

(19) United States
(12) Patent Application Publication
(10) Pub. No.: US 2025/0034582 A1
(43) Pub. Date: Jan. 30, 2025

(54) INCREASING GENE EDITING AND SITE-DIRECTED INTEGRATION EVENTS UTILIZING DEVELOPMENTAL PROMOTERS

(71) Applicant: Monsanto Technology LLC, St. Louis, MO (US)

(72) Inventors: Matthew Bauer, Manchester, MO (US); Brent Delbert Brower-Toland, St. Louis, MO (US); Shunhong Dai, Creve Coeur, MO (US); Brent O'Brien, St. Charles, MO (US); Thomas L. Slewinski, Columbia, MO (US)

(73) Assignee: Monsanto Technology LLC, St. Louis, MO (US)

(21) Appl. No.: 18/903,443

(22) Filed: Oct. 1, 2024

Related U.S. Application Data
(63) Continuation-in-part of application No. 18/191,136, filed on Mar. 28, 2023.
(60) Provisional application No. 63/324,880, filed on Mar. 29, 2022.

(51) Int. Cl. C12N 15/82; C12N 9/22; C12N 15/11

(52) U.S. Cl. CPC C12N 15/8213; C12N 9/22; C12N 15/11; C12N 15/823; C12N 2310/20; C12N 2800/80

(57) ABSTRACT

This disclosure provides methods and compositions for increasing genome editing and site-directed integration events utilizing guided endonucleases and floral cell-preferred or floral tissue-preferred promoters.

### CROSS-REFERENCE TO RELATED APPLICATION

This application is a continuation-in-part of U.S. patent application Ser. No. 18/191,136, filed Mar. 28, 2023, which claims the benefit of U.S. Provisional Application No. 63/324,880, filed Mar. 29, 2022. The entirety of each of the above-referenced applications is incorporated by reference herein.

### FIELD

The present disclosure relates to compositions and methods related to expressing guided nucleases and guide nucleic acids in floral cells and floral tissues in plants.

### INCORPORATION OF SEQUENCE LISTING

A sequence listing contained in the file named “P34740US01_SL.XML” which is 145.3 kilobytes (measured in MS-Windows®), produced on Mar. 8, 2023, and containing 52 sequences, is filed electronically herewith and incorporated by reference in its entirety.

### BACKGROUND

CRISPR (Clustered Regularly Interspaced Short Palindromic Repeats) nucleases (e.g., Cas12a, CasX, Cas9) are proteins guided by guide RNAs to a target nucleic acid molecule, where the nuclease can cleave one or two strands of a target nucleic acid molecule.

### SUMMARY

In one aspect, this disclosure provides a plant comprising (a) a first nucleic acid sequence encoding a guided nuclease capable of generating a staggered cut in a double-stranded DNA molecule operably linked to a heterologous floral tissue-preferred promoter or floral cell-preferred promoter; and (b) a second nucleic acid sequence encoding at least one guide nucleic acid operably linked to a heterologous second promoter, where the at least one guide nucleic acid is capable of hybridizing to a target sequence within a genome of the plant.

In one aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous floral cell-preferred promoter; and (ii) a second nucleic acid sequence encoding at least one guide nucleic acid operably linked to a heterologous second promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one floral cell of the plant, and where the ribonucleoprotein generates at least one double-stranded break within the target sequence in the at least one floral cell.

In one aspect, this disclosure provides a method of editing a genome of a plant cell comprising: (a) crossing a first plant with a second plant, where the first plant comprises a first nucleic acid sequence encoding a guided nuclease capable of generating a staggered cut in a double-stranded DNA molecule operably linked to a heterologous floral tissue-preferred promoter, and where the second plant comprises a second nucleic acid sequence encoding at least one guide nucleic acid operably linked to a heterologous second promoter, where the at least one guide nucleic acid is capable of hybridizing to a target sequence within the genome; and (b) obtaining at least one embryo from the crossing of step (a), where the guided nuclease and the at least one guide nucleic acid form a ribonucleoprotein within the at least one embryo, and where the ribonucleoprotein generates at least one double-stranded break within the target sequence in the at least one embryo.

In one aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous floral tissue-preferred promoter; and (ii) a second nucleic acid sequence encoding at least one guide nucleic acid operably linked to a heterologous second promoter, where the at least one guide nucleic acid is capable of hybridizing to a target sequence within the genome; (b) regenerating at least one plant from the plant cell of step (a); and (c) fertilizing the at least one plant to create at least one embryo, where the guided nuclease and at least one guide nucleic acid form a ribonucleoprotein within the at least one embryo from step (c), and where the ribonucleoprotein generates at least one double-stranded break within the target sequence in the at least one embryo.

In one aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous floral cell-preferred promoter; (ii) a second nucleic acid sequence encoding one or more guide nucleic acids operably linked to a heterologous second promoter, where the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; and (b) regenerating at least one plant from the plant cell of step (a); where the guided nuclease and at least one guide RNA form a ribonucleoprotein within at least one floral cell of the plant, where the ribonucleoprotein generates a double-stranded break within the target sequence, the first site, and the second site, and where the gene of interest is integrated into the target site in the at least one floral cell.

In one aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous floral tissue-preferred promoter; (ii) a second nucleic acid sequence encoding one or more guide nucleic acids operably linked to a heterologous second promoter, where the one or more guide nucleic acids are (A) capable of hybridizing to a target sequence within a genome of the plant; and (B) capable of hybridizing to a first site and a second site flanking a nucleic acid sequence encoding a gene of interest; (iii) a third nucleic acid sequence encoding the gene of interest; (b) regenerating at least one plant from the plant cell of step (a); and (c) fertilizing the at least one plant from step (b) to create at least one embryo; where the guided nuclease and at least one guide RNA form a ribonucleoprotein within at least one embryo, where the ribonucleoprotein generates a double-stranded break within the target sequence, the first site, and the second site, and where the gene of interest is integrated into the target site in the at least one embryo.

In one aspect, this disclosure provides a recombinant DNA construct comprising (a) a first nucleic acid sequence encoding a guided nuclease capable of generating a staggered cut in a double-stranded DNA molecule operably linked to a heterologous floral tissue-preferred promoter; and (b) a second nucleic acid sequence encoding at least one guide nucleic acid operably linked to a heterologous second promoter, where the at least one guide nucleic acid is capable of hybridizing to a target sequence within a genome of a plant.

In one aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous floral cell-preferred promoter and (ii) a second nucleic acid sequence encoding at least one guide nucleic acid operably linked to a heterologous second promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one floral cell of the plant, and where the ribonucleoprotein generates at least one double-stranded break within the target sequence in the at least one floral cell.

In one aspect, this disclosure provides a method of editing a genome of a plant cell comprising: (a) crossing a first plant with a second plant, where the first plant comprises a first nucleic acid sequence encoding a guided nuclease capable of generating a staggered cut in a double-stranded DNA molecule operably linked to a heterologous floral tissue-preferred promoter, and where the second plant comprises a second nucleic acid sequence encoding at least one guide nucleic acid operably linked to a heterologous second promoter, where the at least one guide nucleic acid is capable of hybridizing to a target sequence within the genome; and (b) obtaining at least one progeny plant from the crossing of step (a), where the guided nuclease and the at least one guide nucleic acid form a ribonucleoprotein within at least one floral cell, and where the ribonucleoprotein generates at least one double-stranded break within the target sequence in the at least one floral cell.

In one aspect, this disclosure provides a method of editing a genome of a plant cell comprising (a) crossing a first plant with a second plant, where the first plant comprises a first nucleic acid sequence encoding a guided nuclease capable of generating a staggered cut in a double-stranded DNA molecule operably linked to a heterologous floral cell-preferred promoter, and where the second plant comprises a second nucleic acid sequence encoding at least one guide nucleic acid operably linked to a heterologous second promoter, where the at least one guide nucleic acid is capable of hybridizing to a target sequence within the genome; and (b) obtaining at least one progeny plant from the crossing of step (a), where the guided nuclease and the at least one guide nucleic acid form a ribonucleoprotein within at least one floral cell, and where the ribonucleoprotein generates at least one double-stranded break within the target sequence in the at least one floral cell.

In one aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous floral cell-preferred promoter; (ii) a second nucleic acid sequence encoding one or more guide nucleic acids operably linked to a heterologous second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; and (b) regenerating at least one plant from the plant cell of step (a); where the guided nuclease and at least one guide RNA form a ribonucleoprotein within at least one floral cell of the plant, where the ribonucleoprotein generates a double-stranded break within the target sequence molecule, the first site, and the second site, and where the gene of interest is integrated into the target sequence in the at least one floral cell.

In one aspect, this disclosure provides a method of generating two or more progeny plants with unique edits from a single transformed plant cell, the method comprising: (a) introducing into the plant cell: (i) a first nucleic acid sequence encoding a guided nuclease capable of generating a staggered cut in a double-stranded DNA molecule operably linked to a heterologous floral cell-preferred promoter; and (ii) a second nucleic acid sequence encoding at least one guide nucleic acid operably linked to a heterologous second promoter, where the at least one guide nucleic acid is capable of hybridizing to a target sequence within the genome; and (b) regenerating a first plant from the plant cell of step (a), where the guided nuclease and at least one guide nucleic acid form a ribonucleoprotein within at least one floral cell of the first plant, and where the ribonucleoprotein generates at least one double-stranded break within the target sequence in the at least one floral cell; (c) pollinating the first plant of step (b); and (d) germinating two or more seeds produced from step (c) to produce two or more progeny plants with unique edits.

In one aspect, this disclosure provides a method of generating two or more progeny plants with unique edits from a single transformed plant cell, the method comprising: (a) introducing into the plant cell: (i) a first nucleic acid sequence encoding a guided nuclease capable of generating a staggered cut in a double-stranded DNA molecule operably linked to a heterologous first promoter; and (ii) a second nucleic acid sequence encoding at least one guide nucleic acid operably linked to a heterologous floral cell-preferred promoter, where the at least one guide nucleic acid is capable of hybridizing to a target sequence within the genome; and (b) regenerating a first plant from the plant cell of step (a), where the guided nuclease and at least one guide nucleic acid form a ribonucleoprotein within at least one floral cell of the first plant, and where the ribonucleoprotein generates at least one double-stranded break within the target sequence in the at least one floral cell; (c) pollinating the first plant of step (b); and (d) germinating two or more seeds produced from step (c) to produced two or more progeny plants with unique edits.

In one aspect, this disclosure provides a method of generating two or more progeny plants with unique edits from a single transformed plant cell, the method comprising: (a) introducing into the plant cell: (i) a first nucleic acid sequence encoding a guided nuclease capable of generating a staggered cut in a double-stranded DNA molecule operably linked to a heterologous floral tissue-preferred promoter; and (ii) a second nucleic acid sequence encoding at least one guide nucleic acid operably linked to a heterologous second promoter, where the at least one guide nucleic acid is capable of hybridizing to a target sequence within the genome; and (b) regenerating a first plant from the plant cell of step (a); (c) pollinating the first plant of step (b), where the guided nuclease and at least one guide nucleic acid form a ribonucleoprotein within a floral tissue, and where the ribonucleoprotein generates at least one double-stranded break within the target sequence in the floral tissue; and (d) germinating two or more seeds produced from step (c) to produced two or more progeny plants with unique edits.

In one aspect, this disclosure provides a method of generating two or more progeny plants with unique edits from a single transformed plant cell, the method comprising: (a) introducing to the plant cell: (i) a first nucleic acid sequence encoding a guided nuclease capable of generating a staggered cut in a double-stranded DNA molecule operably linked to a heterologous first promoter; and; (ii) a second nucleic acid sequence encoding at least one guide nucleic acid operably linked to a heterologous floral tissue-preferred promoter, wherein the at least one guide nucleic acid is capable of hybridizing to a target sequence within the genome; and (b) regenerating a first plant from the plant cell of step (a); (c) pollinating the first plant of step (b), wherein the guided nuclease and at least one guide nucleic acid form a ribonucleoprotein within a floral tissue, and wherein the ribonucleoprotein generates at least one double-stranded break within the target sequence in the floral tissue; and (d) germinating two or more seeds produced from step (c) to produced two or more progeny plants with unique edits.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

As used herein, terms in the singular and the singular forms “a,” “an,” and “the,” for example, include plural referents unless the content clearly dictates otherwise.

Any composition, nucleic acid molecule, polypeptide, cell, plant, etc. provided herein is specifically envisioned for use with any method provided herein.

In an aspect, this disclosure provides a recombinant DNA construct comprising a first nucleic acid sequence encoding a guided nuclease capable of generating a staggered cut in a double-stranded DNA molecule operably linked to a heterologous floral tissue-preferred promoter; and (b) a second nucleic acid sequence encoding at least one guide nucleic acid operably linked to a heterologous second promoter, where the at least one guide nucleic acid is capable of hybridizing to a target sequence within a genome of a plant.

In an aspect, this disclosure provides a recombinant DNA construct comprising a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to a floral tissue-preferred promoter, such as a heterologous floral tissue-preferred promoter, where the at least one guide nucleic acid is capable of hybridizing to a target sequence within a genome of a plant.

In an aspect, this disclosure provides a plant comprising a recombinant DNA construct comprising a first nucleic acid sequence encoding a guided nuclease capable of generating a staggered cut in a double-stranded DNA molecule operably linked to a heterologous floral tissue-preferred promoter; and (b) a second nucleic acid sequence encoding at least one guide nucleic acid operably linked to a heterologous second promoter, where the at least one guide nucleic acid is capable of hybridizing to a target sequence within a genome of the plant.

In an aspect, this disclosure provides a plant comprising a recombinant DNA construct comprising a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an floral tissue-preferred promoter, where the at least one guide nucleic acid is capable of hybridizing to a target sequence within a genome of the plant.

In an aspect, this disclosure provides a plant comprising (a) a first nucleic acid sequence encoding a guided nuclease capable of generating a staggered cut in a double-stranded DNA molecule operably linked to a heterologous floral tissue-preferred promoter or floral cell-preferred promoter; and (b) a second nucleic acid sequence encoding at least one guide nucleic acid operably linked to a heterologous second promoter, where the at least one guide nucleic acid is capable of hybridizing to a target sequence within a genome of the plant. In an aspect, this disclosure provides a plant comprising (a) a first nucleic acid sequence encoding a guided nuclease capable of generating a staggered cut in a double-stranded DNA molecule operably linked to a first promoter; and (b) a second nucleic acid sequence encoding at least one guide nucleic acid operably linked to a heterologous floral tissue-preferred promoter or floral cell-preferred promoter, where the at least one guide nucleic acid is capable of hybridizing to a target sequence within a genome of the plant.

In an aspect, this disclosure provides a seed of any plant provided herein.

As used herein, “floral tissue” refers to any tissue or cell that gives rise to any part of a flower, excluding a shoot apical meristem. Non-limiting examples of floral tissue include branch meristems, axillary meristems, inflorescence meristems, floral meristems, lemmas, paleas, lodicules, peduncles, receptacles, sepals, petals, stigmas, styles, filaments, anthers. As used herein, “floral tissue” does not refer to ovaries, ovules or pollen.

In an aspect, a floral tissue comprises a structure selected from the group consisting of a branch meristem, an axillary meristem, an inflorescence meristem, a floral meristem, a lemma, a palea, a lodicule, a peduncle, a receptacle, a sepal, a petal, a stigma, a style, a filament, and an anther

When plants transition from vegetative growth to flowering, the shoot apical meristem is transformed into an inflorescence meristem. The inflorescence meristem then produces floral meristems, which ultimately give rise to all of the tissues of a flower, including, without being limiting, lemmas, paleas, lodicules, receptacles, sepals, petals, stigmas, styles, filaments and anthers. In an aspect, a floral tissue comprises an inflorescence meristem. In an aspect, a floral tissue comprises a floral meristem. In an aspect, a floral tissue comprises a branch meristem. In grasses, branch meristems are produced by an inflorescence meristem, and the branch meristems produce branches or spikelets in two ranks to pattern floral organs in a whorled phyllotaxis.

In an aspect, a floral tissue comprises a peduncle. In an aspect, a floral tissue comprises a lemma. In an aspect, a floral tissue comprises a palea. In an aspect, a floral tissue comprises a lodicule. In an aspect, a floral tissue comprises a receptacle. In an aspect, a floral tissue comprises a sepal. In an aspect, a floral tissue comprises a petal. In an aspect, a floral tissue comprises a stigma. In an aspect, a floral tissue comprises a style. In an aspect, a floral tissue comprises a filament. In an aspect, a floral tissue comprises an anther.

In an aspect, a floral tissue does not comprise a shoot apical meristem. In an aspect, a floral cell does not comprise a shoot apical meristem cell. In an aspect, a floral tissue does not comprise an ovary. In an aspect, a floral tissue does not comprise an ovule. In an aspect, a floral tissue does not comprise pollen.

As used herein, a “floral cell” refers to a cell of any floral tissue. In an aspect, a floral cell is a branch meristem cell. In an aspect, a floral cell is an inflorescence meristem cell. In an aspect, a floral cell is a floral meristem cell. In an aspect, a floral cell is a peduncle cell. In an aspect, a floral cell is a lemma cell. In an aspect, a floral cell is a palea cell. In an aspect, a floral cell is a lodicule cell. In an aspect, a floral cell is a receptacle cell. In an aspect, a floral cell is a sepal cell. In an aspect, a floral cell is a petal cell. In an aspect, a floral cell is a stigma cell. In an aspect, a floral cell is a style cell. In an aspect, a floral cell is a filament cell. In an aspect, a floral cell is an anther cell.

### Nucleic Acids and Amino Acids

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise deoxyribonucleotides, ribonucleotides, or combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded.

As used herein, the term “recombinant” in reference to a nucleic acid (DNA or RNA) molecule, protein, construct, vector, etc., refers to a nucleic acid or amino acid molecule or sequence that is man-made and not normally found in nature, and/or is present in a context in which it is not normally found in nature, including a nucleic acid molecule (DNA or RNA) molecule, protein, construct, etc., comprising a combination of polynucleotide or protein sequences that would not naturally occur contiguously or in close proximity together without human intervention, and/or a polynucleotide molecule, protein, construct, etc., comprising at least two polynucleotide or protein sequences that are heterologous with respect to each other.

In one aspect, methods and compositions provided herein comprise a vector. As used herein, the term “vector” refers to a DNA molecule used as a vehicle to carry exogenous genetic material into a cell.

In an aspect, one or more polynucleotide sequences from a vector are stably integrated into a genome of a plant. In an aspect, one or more polynucleotide sequences from a vector are not stably integrated into a genome of a plant cell.

In an aspect, a first nucleic acid sequence and a second nucleic acid sequence are provided in a single vector. In another aspect, a first nucleic acid sequence is provided in a first vector, and a second nucleic acid sequence is provided in a second vector.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

The terms “percent sequence complementarity” or “percent complementarity” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

For optimal alignment of sequences to calculate their percent identity, various pair-wise or multiple sequence alignment algorithms and programs are known in the art, such as ClustalW or Basic Local Alignment Search Tool (BLAST®), etc., that can be used to compare the sequence identity or similarity between two or more nucleotide or protein sequences. Although other alignment and comparison methods are known in the art, the alignment and percent identity between two sequences (including the percent identity ranges described above) can be as determined by the ClustalW algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine pairing with thymine, adenine pairing with uracil, and guanine (G) pairing with cytosine (C) [DNA, RNA]. In addition, it is also known in the art that for hybridization between two RNA molecules (e.g., dsRNA), guanine base pairs with uracil. For example, G/U base-pairing is partially responsible for the degeneracy (i.e., redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et al.). Typically, the length for a hybridizable nucleic acid is at least 10 nucleotides. Illustrative minimum lengths for a hybridizable nucleic acid are: at least 15 nucleotides; at least 18 nucleotides; at least 20 nucleotides; at least 22 nucleotides; at least 25 nucleotides; and at least 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Generating Edits

In an aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous floral tissue-preferred promoter; and (b) a second nucleic acid sequence encoding at least one guide nucleic acid operably linked to a heterologous second promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one floral cell of the plant, and where the ribonucleoprotein generates at least one double-stranded break within the target sequence in the at least one floral cell.

In an aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to a heterologous floral tissue-preferred promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one floral cell of the plant, and where the ribonucleoprotein generates at least one double-stranded break within the target sequence in the at least one floral cell.

In an aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous floral cell-preferred promoter; and (b) a second nucleic acid sequence encoding at least one guide nucleic acid operably linked to a heterologous second promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one floral cell of the plant, and where the ribonucleoprotein generates at least one double-stranded break within the target sequence in the at least one floral cell.

In an aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an floral cell-preferred promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one floral cell of the plant, and where the ribonucleoprotein generates at least one double-stranded break within the target sequence in the at least one floral cell.

In an aspect, this disclosure provides a method of editing a genome of a plant cell comprising: (a) crossing a first plant with a second plant, where the first plant comprises a first nucleic acid sequence encoding a guided nuclease capable of generating a staggered cut in a double-stranded DNA molecule operably linked to a heterologous floral tissue-preferred promoter, and where the second plant comprises a second nucleic acid sequence encoding at least one guide nucleic acid operably linked to a heterologous second promoter, wherein the at least one guide nucleic acid is capable of hybridizing to a target sequence within the genome; and (b) obtaining at least one embryo from the crossing of step (a), where the guided nuclease and the at least one guide nucleic acid form a ribonucleoprotein within the at least one embryo, and where the ribonucleoprotein generates at least one double-stranded break within the target sequence in the at least one embryo.

In an aspect, this disclosure provides a method of editing a genome of a plant cell comprising: (a) crossing a first plant with a second plant, where the first plant comprises a first nucleic acid sequence encoding a guided nuclease capable of generating a staggered cut in a double-stranded DNA molecule operably linked to a heterologous promoter, and where the second plant comprises a second nucleic acid sequence encoding at least one guide nucleic acid operably linked to a floral tissue-preferred promoter, wherein the at least one guide nucleic acid is capable of hybridizing to a target sequence within the genome; and (b) obtaining at least one embryo from the crossing of step (a), where the guided nuclease and the at least one guide nucleic acid form a ribonucleoprotein within the at least one embryo, and where the ribonucleoprotein generates at least one double-stranded break within the target sequence in the at least one embryo.

In an aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous floral tissue-preferred promoter; and (ii) a second nucleic acid sequence encoding at least one guide nucleic acid operably linked to a heterologous second promoter, where the at least one guide nucleic acid is capable of hybridizing to a target sequence within the genome; (b) regenerating at least one plant from the plant cell of step (a); and (c) fertilizing the at least one plant to create at least one embryo, where the guided nuclease and at least one guide nucleic acid form a ribonucleoprotein within the at least one embryo from step (c), and where the ribonucleoprotein generates at least one double-stranded break within the target sequence in the at least one embryo.

In an aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous promoter; and (ii) a second nucleic acid sequence encoding at least one guide nucleic acid operably linked to a floral tissue-preferred promoter, where the at least one guide nucleic acid is capable of hybridizing to a target sequence within the genome; (b) regenerating at least one plant from the plant cell of step (a); and (c) fertilizing the at least one plant to create at least one embryo, where the guided nuclease and at least one guide nucleic acid form a ribonucleoprotein within the at least one embryo from step (c), and where the ribonucleoprotein generates at least one double-stranded break within the target sequence in the at least one embryo.

In an aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous floral tissue-preferred promoter; and (ii) a second nucleic acid sequence encoding one or more guide nucleic acids operably linked to a heterologous second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); where the guided nuclease and at least one guide RNA form a ribonucleoprotein within at least one floral cell of the plant, where the ribonucleoprotein generates a double-stranded break within the target sequence, the first site, and the second site, and where the gene of interest is integrated into the target site in the at least one floral cell.

In an aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous promoter; and (ii) a second nucleic acid sequence encoding one or more guide nucleic acids operably linked to a floral tissue-preferred second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); where the guided nuclease and at least one guide RNA form a ribonucleoprotein within at least one floral cell of the plant, where the ribonucleoprotein generates a double-stranded break within the target sequence, the first site, and the second site, and where the gene of interest is integrated into the target site in the at least one floral cell.

In an aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous floral cell-preferred promoter; and (ii) a second nucleic acid sequence encoding one or more guide nucleic acids operably linked to a heterologous second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); where the guided nuclease and at least one guide RNA form a ribonucleoprotein within at least one floral cell of the plant, where the ribonucleoprotein generates a double-stranded break within the target sequence, the first site, and the second site, and where the gene of interest is integrated into the target site in the at least one floral cell.

In an aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous promoter; and (ii) a second nucleic acid sequence encoding one or more guide nucleic acids operably linked to a floral cell-preferred second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); where the guided nuclease and at least one guide RNA form a ribonucleoprotein within at least one floral cell of the plant, where the ribonucleoprotein generates a double-stranded break within the target sequence, the first site, and the second site, and where the gene of interest is integrated into the target site in the at least one floral cell.

In an aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous floral tissue-preferred promoter; (ii) a second nucleic acid sequence encoding one or more guide nucleic acids operably linked to a heterologous second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); and (c) fertilizing the at least one plant from step (b) to create at least one embryo; where the guided nuclease and at least one guide RNA form a ribonucleoprotein within at least one embryo, where the ribonucleoprotein generates a double-stranded break within the target sequence, the first site, and the second site, and where the gene of interest is integrated into the target site in the at least one embryo.

In an aspect, this disclosure provides a method of generating a site-directed integration in a plant comprising: (a) introducing into a plant cell: (i) a first nucleic acid sequence encoding a guided nuclease capable of generating a staggered cut in a double-stranded DNA molecule operably linked to a heterologous promoter; (ii) a second nucleic acid sequence encoding one or more guide nucleic acids operably linked to a floral tissue-preferred promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); and (c) fertilizing the at least one plant from step (b) to create at least one embryo; where the guided nuclease and at least one guide RNA form a ribonucleoprotein within at least one embryo, where the ribonucleoprotein generates a double-stranded break within the target sequence, the first site, and the second site, and where the gene of interest is integrated into the target site in the at least one embryo.

In an aspect, this disclosure provides a method of editing a genome of a plant comprising: (a) introducing into a plant cell: (i) a first nucleic acid sequence encoding a guided nuclease capable of generating a staggered cut in a double-stranded DNA molecule operably linked to a heterologous floral cell-preferred promoter and (ii) a second nucleic acid sequence encoding at least one guide nucleic acid operably linked to a heterologous second promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one floral cell of the plant, and where the ribonucleoprotein generates at least one double-stranded break within the target sequence in the at least one floral cell.

In an aspect, this disclosure provides a method of editing a genome of a plant cell comprising: (a) crossing a first plant with a second plant, where the first plant comprises a first nucleic acid sequence encoding a guided nuclease capable of generating a staggered cut in a double-stranded DNA molecule operably linked to a heterologous floral tissue-preferred promoter, and where the second plant comprises a second nucleic acid sequence encoding at least one guide nucleic acid operably linked to a heterologous second promoter, where the at least one guide nucleic acid is capable of hybridizing to a target sequence within the genome; and (b) obtaining at least one progeny plant from the crossing of step (a), where the guided nuclease and the at least one guide nucleic acid form a ribonucleoprotein within at least one floral cell, and where the ribonucleoprotein generates at least one double-stranded break within the target sequence in the at least one floral cell.

In an aspect, this disclosure provides a method of editing a genome of a plant cell comprising (a) crossing a first plant with a second plant, where the first plant comprises a first nucleic acid sequence encoding a guided nuclease capable of generating a staggered cut in a double-stranded DNA molecule operably linked to a heterologous floral cell-preferred promoter, and where the second plant comprises a second nucleic acid sequence encoding at least one guide nucleic acid operably linked to a heterologous second promoter, where the at least one guide nucleic acid is capable of hybridizing to a target sequence within the genome; and (b) obtaining at least one progeny plant from the crossing of step (a), where the guided nuclease and the at least one guide nucleic acid form a ribonucleoprotein within at least one floral cell, and where the ribonucleoprotein generates at least one double-stranded break within the target sequence in the at least one floral cell.

In an aspect, this disclosure provides a method of generating a site-directed integration in a plant comprising: (a) introducing to a plant cell: (i) a first nucleic acid sequence encoding a guided nuclease capable of generating a staggered cut in a double-stranded DNA molecule operably linked to a heterologous floral cell-preferred promoter; (ii) a second nucleic acid sequence encoding one or more guide nucleic acids operably linked to a heterologous second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; and (b) regenerating at least one plant from the plant cell of step (a); where the guided nuclease and at least one guide RNA form a ribonucleoprotein within at least one floral cell of the plant, where the ribonucleoprotein generates a double-stranded break within the target sequence molecule, the first site, and the second site, and where the gene of interest is integrated into the target sequence in the at least one floral cell.

In an aspect, this disclosure provides a method of generating two or more progeny plants with unique edits from a single transformed plant cell, the method comprising: (a) introducing to the plant cell: (i) a first nucleic acid sequence encoding a guided nuclease capable of generating a staggered cut in a double-stranded DNA molecule operably linked to a heterologous floral cell-preferred promoter; and (ii) a second nucleic acid sequence encoding at least one guide nucleic acid operably linked to a heterologous second promoter, where the at least one guide nucleic acid is capable of hybridizing to a target sequence within the genome; and (b) regenerating a first plant from the plant cell of step (a), where the guided nuclease and at least one guide nucleic acid form a ribonucleoprotein within at least one floral cell of the first plant, and where the ribonucleoprotein generates at least one double-stranded break within the target sequence in the at least one floral cell; (c) pollinating the first plant of step (b); and (d) germinating two or more seeds produced from step (c) to produce two or more progeny plants with unique edits.

In an aspect, this disclosure provides a method of generating two or more progeny plants with unique edits from a single transformed plant cell, the method comprising: (a) introducing to the plant cell: (i) a first nucleic acid sequence encoding a guided nuclease capable of generating a staggered cut in a double-stranded DNA molecule operably linked to a heterologous first promoter; and (ii) a second nucleic acid sequence encoding at least one guide nucleic acid operably linked to a heterologous floral cell-preferred promoter, where the at least one guide nucleic acid is capable of hybridizing to a target sequence within the genome; and (b) regenerating a first plant from the plant cell of step (a), where the guided nuclease and at least one guide nucleic acid form a ribonucleoprotein within at least one floral cell of the first plant, and where the ribonucleoprotein generates at least one double-stranded break within the target sequence in the at least one floral cell; (c) pollinating the first plant of step (b); and (d) germinating two or more seeds produced from step (c) to produced two or more progeny plants with unique edits.

In an aspect, this disclosure provides a method of generating two or more progeny plants with unique edits from a single transformed plant cell, the method comprising: (a) introducing to the plant cell: (i) a first nucleic acid sequence encoding a guided nuclease capable of generating a staggered cut in a double-stranded DNA molecule operably linked to a heterologous floral tissue-preferred promoter; and (ii) a second nucleic acid sequence encoding at least one guide nucleic acid operably linked to a heterologous second promoter, where the at least one guide nucleic acid is capable of hybridizing to a target sequence within the genome; and (b) regenerating a first plant from the plant cell of step (a); (c) pollinating the first plant of step (b), where the guided nuclease and at least one guide nucleic acid form a ribonucleoprotein within a floral tissue, and where the ribonucleoprotein generates at least one double-stranded break within the target sequence in the floral tissue; and (d) germinating two or more seeds produced from step (c) to produced two or more progeny plants with unique edits.

In an aspect, this disclosure provides a method of generating two or more progeny plants with unique edits from a single transformed plant cell, the method comprising: (a) introducing to the plant cell: (i) a first nucleic acid sequence encoding a guided nuclease capable of generating a staggered cut in a double-stranded DNA molecule operably linked to a heterologous first promoter; and; (ii) a second nucleic acid sequence encoding at least one guide nucleic acid operably linked to a heterologous floral tissue-preferred promoter, wherein the at least one guide nucleic acid is capable of hybridizing to a target sequence within the genome; and (b) regenerating a first plant from the plant cell of step (a); (c) pollinating the first plant of step (b), wherein the guided nuclease and at least one guide nucleic acid form a ribonucleoprotein within a floral tissue, and wherein the ribonucleoprotein generates at least one double-stranded break within the target sequence in the floral tissue; and (d) germinating two or more seeds produced from step (c) to produced two or more progeny plants with unique edits.

### Promoters

As commonly understood in the art, the term “promoter” refers to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also comprise leaders, 5′ UTRs and introns. A promoter can also include a chimeric promoter comprising a combination of two or more heterologous sequences A promoter of the present application can thus include variants of promoter sequences that are similar in composition, but not identical to, other promoter sequence(s) known or provided herein. A promoter can be classified according to a variety of criteria relating to the pattern of expression of an associated coding or transcribable sequence or gene (including a transgene) operably linked to the promoter, such as constitutive, developmental, tissue-specific, cell cycle-specific, inducible, etc.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous.

In an aspect, a recombinant nucleic acid provided herein comprises at least one promoter. In another aspect, a polynucleotide encoding a guided nuclease is operably linked to at least one promoter. In another aspect, a polynucleotide encoding a Cas12a nuclease is operably linked to at least one promoter. In another aspect, a polynucleotide encoding a CasX nuclease is operably linked to at least one promoter. In another aspect, a polynucleotide encoding MAD7® nuclease is operably linked to at least one promoter. In another aspect, a polynucleotide encoding a guide nucleic acid is operably linked to at least one promoter.

Promoters that express within a specific tissue(s) of an organism, with no expression in other tissues, are referred to as “tissue-specific” promoters. Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. In another aspect, a promoter provided herein is a tissue-specific promoter. In a further aspect, a promoter provided herein is a tissue-preferred promoter. In an aspect, a tissue-preferred promoter comprises a tissue-specific promoter.

Determination of promoter activity can be performed using any method standard in the art. For example, without being limiting, a promoter of interest can be used to drive expression of a fluorophore or other reporting molecule, and the concentration of the expressed molecule can be used to determine promoter activity in different cell or tissue types.

Flowers are organized into concentric whorls of sepals, petals, stamens (e.g., a structure comprising a filament and an anther) and carpels (e.g. a structure comprising an ovary, a stigma, and often a style), with each of these floral organ types having a unique role in reproduction. Genes involved in establishing floral architecture are homeotic MADS-domain transcription factors (with the exception of the A gene APATELA2, see below) that are classified as A genes, B genes, C genes, D genes, or E genes by the ABCDE model of floral development, which has been used to describe how floral architecture is genetically specified. See, for example, Coen and Meyerowitz, Nature, 353:31-37 (1991); and Murai, Plants, 2:379-395 (2013). Under the ABCDE model, sepals are formed when A and E genes are co-expressed; carpels are formed when C and E genes are co-expressed; petals are formed when A, B, and E genes are co-expressed; stamens are formed when B, C, and E genes are co-expressed; and ovules are formed when D and E genes are co-expressed.

Non-limiting examples of A genes in Arabidopsis and soybean include APETALA1 (AP1) and APETALA2 (AP2), which is an ERENUCLEOTIDE transcription factor. Non-limiting examples of B genes in Arabidopsis and soybean include APETALA3 (AP3) and PISTILLATA (PI). A non-limiting example of a C gene in Arabidopsis and soybean is AGAMOUS (AG). Non-limiting examples of D genes in Arabidopsis and soybean include AGAMOUS-LIKE 11/SEEDSTICK (AGL11/STK), AGAMOUS-LIKE 1/SHATTERPROOF1 (AGL1/SHP1), AGAMOUS-LIKE 5/SHATTERPROOF2 (AGL5/SHP2). Non-limiting examples of E genes in Arabidopsis and soybean include SEPALLATA1 (SEP1), SEPALLATA2 (SEP2), SEPALLATA3 (SEP3), AND SEPALLATA4 (SEP4).

Non-limiting examples of an A gene in corn include ZEAAPETALA HOMOLOG1 (ZAP1). Non-limiting examples of B genes in corn include ZEA MAYS MADS16 (ZMM16) and ZEA MAYS MADS18 (ZMM18). Non-limiting examples of C genes in corn include ZEA AGAMOUS HOMOLOG1 (ZAG1), ZEA MAYS MADS2 (ZMM2), and ZEA MAYS MADS23 (ZMM23). Non-limiting examples of D genes in corn include ZEA AGAMOUS HOMOLOG2 (ZAG2) AND ZEA MAYS MADS1 (ZMM1). Non-limiting examples of E genes in corn include ZEA AGAMOUS HOMOLOG3 (ZAG3) AND ZEA MAYS MADS7/SEPALLATA-LIKE (ZMM7/SEP-like).

In an aspect, a floral tissue-preferred promoter is an A gene promoter. In an aspect, a floral tissue-preferred promoter is a B gene promoter. In an aspect, a floral tissue-preferred promoter is a C gene promoter. In an aspect, a floral tissue-preferred promoter is a D gene promoter. In an aspect, a floral tissue-preferred promoter is an E gene promoter.

In an aspect, a floral cell-preferred promoter is an A gene promoter. In an aspect, a floral cell-preferred promoter is a B gene promoter. In an aspect, a floral cell-preferred promoter is a C gene promoter. In an aspect, a floral cell-preferred promoter is a D gene promoter. In an aspect, a floral cell-preferred promoter is an E gene promoter.

In an aspect, a floral tissue-preferred promoter comprises a promoter selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

In an aspect, a floral tissue-preferred promoter comprises an AP1 promoter. In an aspect, a floral tissue-preferred promoter comprises an AP2 promoter. In an aspect, a floral tissue-preferred promoter comprises a ZAP1 promoter. In an aspect, a floral tissue-preferred promoter comprises an AP3 promoter. In an aspect, a floral tissue-preferred promoter comprises a PI promoter. In an aspect, a floral tissue-preferred promoter comprises a ZMM16 promoter. In an aspect, a floral tissue-preferred promoter comprises a ZMM18 promoter. In an aspect, a floral tissue-preferred promoter comprises an AG promoter. In an aspect, a floral tissue-preferred promoter comprises a ZAG1 promoter. In an aspect, a floral tissue-preferred promoter comprises a ZMM2 promoter. In an aspect, a floral tissue-preferred promoter comprises a ZMM23 promoter. In an aspect, a floral tissue-preferred promoter comprises an AGL11/STK promoter. In an aspect, a floral tissue-preferred promoter comprises an AGL1/SHP1 promoter. In an aspect, a floral tissue-preferred promoter comprises an AGL5/SHP2 promoter. In an aspect, a floral tissue-preferred promoter comprises a ZAG2 promoter. In an aspect, a floral tissue-preferred promoter comprises a ZMM1 promoter. In an aspect, a floral tissue-preferred promoter comprises a SEP1 promoter. In an aspect, a floral tissue-preferred promoter comprises a SEP2 promoter. In an aspect, a floral tissue-preferred promoter comprises a SEP3 promoter. In an aspect, a floral tissue-preferred promoter comprises a SEP4 promoter. In an aspect, a floral tissue-preferred promoter comprises a ZAG3 promoter. In an aspect, a floral tissue-preferred promoter comprises a ZMM7/SEP-like promoter.

In an aspect, a floral cell-preferred promoter comprises a promoter selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

In an aspect, a floral cell-preferred promoter comprises an AP1 promoter. In an aspect, a floral cell-preferred promoter comprises an AP2 promoter. In an aspect, a floral cell-preferred promoter comprises a ZAP1 promoter. In an aspect, a floral cell-preferred promoter comprises an AP3 promoter. In an aspect, a floral cell-preferred promoter comprises a PI promoter. In an aspect, a floral cell-preferred promoter comprises a ZMM16 promoter. In an aspect, a floral cell-preferred promoter comprises a ZMM18 promoter. In an aspect, a floral cell-preferred promoter comprises an AG promoter. In an aspect, a floral cell-preferred promoter comprises a ZAG1 promoter. In an aspect, a floral cell-preferred promoter comprises a ZMM2 promoter. In an aspect, a floral cell-preferred promoter comprises a ZMM23 promoter. In an aspect, a floral cell-preferred promoter comprises an AGL11/STK promoter. In an aspect, a floral cell-preferred promoter comprises an AGL1/SHP1 promoter. In an aspect, a floral cell-preferred promoter comprises an AGL5/SHP2 promoter. In an aspect, a floral cell-preferred promoter comprises a ZAG2 promoter. In an aspect, a floral cell-preferred promoter comprises a ZMM1 promoter. In an aspect, a floral cell-preferred promoter comprises a SEP1 promoter. In an aspect, a floral cell-preferred promoter comprises a SEP2 promoter. In an aspect, a floral cell-preferred promoter comprises a SEP3 promoter. In an aspect, a floral cell-preferred promoter comprises a SEP4 promoter. In an aspect, a floral cell-preferred promoter comprises a ZAG3 promoter. In an aspect, a floral cell-preferred promoter comprises a ZMM7/SEP-like promoter.

In an aspect, a floral tissue-preferred promoter comprises a sequence at least 70% identical to a sequence selected from the group consisting of SEQ ID NOs: 1-30 or selected from the group of SEQ ID NOs: 1-16, 18-19, 21-30 and 45-49, or a functional fragment thereof. In an aspect, a floral tissue-preferred promoter comprises a sequence at least 75% identical to a sequence selected from the group consisting of SEQ ID NOs: 1-30, or selected from the group of SEQ ID NOs: 1-16, 18-19, 21-30 and 45-49 or a functional fragment thereof. In an aspect, a floral tissue-preferred promoter comprises a sequence at least 80% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral tissue-preferred promoter comprises a sequence at least 85% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral tissue-preferred promoter comprises a sequence at least 90% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral tissue-preferred promoter comprises a sequence at least 95% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral tissue-preferred promoter comprises a sequence at least 99% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral tissue-preferred promoter comprises a sequence 100% identical to a sequence selected from the group consisting of SEQ ID NOs: 1-30, or selected from the group of SEQ ID NOs: 1-16, 18-19, 21-30 and 45-49, or a functional fragment thereof.

In an aspect, a floral cell-preferred promoter comprises a sequence at least 70% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral cell-preferred promoter comprises a sequence at least 75% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral cell-preferred promoter comprises a sequence at least 80% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral cell-preferred promoter comprises a sequence at least 85% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral cell-preferred promoter comprises a sequence at least 90% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral cell-preferred promoter comprises a sequence at least 95% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral cell-preferred promoter comprises a sequence at least 99% identical to a sequence selected from the group consisting of SEQ ID NOs: 1-30, or selected from the group of SEQ ID NOs: 1-16, 18-19, 21-30 and 45-49, or a functional fragment thereof. In an aspect, a floral cell-preferred promoter comprises a sequence 100% identical to a sequence selected from the group consisting of SEQ ID NOs: 1-30, or selected from the group of SEQ ID NOs: 1-16, 18-19, 21-30 and 45-49, or a functional fragment thereof.

In an aspect, a promoter provided herein is operably linked to a nucleic acid encoding a guided nuclease. In an aspect, promoter provided herein is operably linked to a nucleic acid encoding a Cas12a nuclease. In an aspect, a promoter provided herein is operably linked to a nucleic acid encoding a CasX nuclease. In an aspect, a promoter provided herein is operably linked to a nucleic acid encoding a MAD7® nuclease. In an aspect, a promoter provided herein is operably linked to a nucleic acid encoding a guide nucleic acid. In an aspect, a promoter provided herein is operably linked to a nucleic acid encoding a guide RNA. In an aspect, a promoter provided herein is operably linked to a nucleic acid encoding a single-guide RNA.

In an aspect, a floral tissue-preferred promoter provided herein is operably linked to a nucleic acid encoding a guided nuclease. In an aspect, a floral tissue-preferred promoter provided herein is operably linked to a nucleic acid encoding a Cas12a nuclease. In an aspect, a floral tissue-preferred promoter provided herein is operably linked to a nucleic acid encoding a CasX nuclease. In an aspect, a floral tissue-preferred promoter provided herein is operably linked to a nucleic acid encoding a MAD7® nuclease. In an aspect, a floral tissue-preferred promoter provided herein is operably linked to a nucleic acid encoding a guide nucleic acid. In an aspect, a floral tissue-preferred promoter provided herein is operably linked to a nucleic acid encoding a guide RNA. In an aspect, a floral tissue-preferred promoter provided herein is operably linked to a nucleic acid encoding a single-guide RNA.

In an aspect, a floral tissue-specific promoter provided herein is operably linked to a nucleic acid encoding a guided nuclease. In an aspect, a floral tissue-specific promoter provided herein is operably linked to a nucleic acid encoding a Cas12a nuclease. In an aspect, a floral tissue-specific promoter provided herein is operably linked to a nucleic acid encoding a CasX nuclease. In an aspect, a floral tissue-specific promoter provided herein is operably linked to a nucleic acid encoding a MAD7® nuclease In an aspect, a floral tissue-specific promoter provided herein is operably linked to a nucleic acid encoding a guide nucleic acid. In an aspect, a floral tissue-specific promoter provided herein is operably linked to a nucleic acid encoding a guide RNA. In an aspect, a floral tissue-specific promoter provided herein is operably linked to a nucleic acid encoding a single-guide RNA.

In an aspect, a floral cell-preferred promoter provided herein is operably linked to a nucleic acid encoding a guided nuclease. In an aspect, a floral cell-preferred promoter provided herein is operably linked to a nucleic acid encoding a Cas12a nuclease. In an aspect, a floral cell-preferred promoter provided herein is operably linked to a nucleic acid encoding a CasX nuclease. In an aspect, a floral cell-preferred promoter provided herein is operably linked to a nucleic acid encoding a MAD7® nuclease In an aspect, a floral cell-preferred promoter provided herein is operably linked to a nucleic acid encoding a guide nucleic acid. In an aspect, a floral cell-preferred promoter provided herein is operably linked to a nucleic acid encoding a guide RNA. In an aspect, a floral cell-preferred promoter provided herein is operably linked to a nucleic acid encoding a single-guide RNA.

In an aspect, a floral cell-specific promoter provided herein is operably linked to a nucleic acid encoding a guided nuclease. In an aspect, a floral cell-specific promoter provided herein is operably linked to a nucleic acid encoding a Cas12a nuclease. In an aspect, a floral cell-specific promoter provided herein is operably linked to a nucleic acid encoding a CasX nuclease. In an aspect, a floral cell-specific promoter provided herein is operably linked to a nucleic acid encoding a MAD7® nuclease In an aspect, a floral cell-specific promoter provided herein is operably linked to a nucleic acid encoding a guide nucleic acid. In an aspect, a floral cell-specific promoter provided herein is operably linked to a nucleic acid encoding a guide RNA. In an aspect, a floral cell-specific promoter provided herein is operably linked to a nucleic acid encoding a single-guide RNA.

In an aspect, an A gene promoter is operably linked to a nucleic acid encoding a guided nuclease. In an aspect, an A gene promoter is operably linked to a nucleic acid encoding a Cas12a nuclease. In an aspect, an A gene promoter is operably linked to a nucleic acid encoding a CasX nuclease. In an aspect, an A gene promoter is operably linked to a nucleic acid encoding a MAD7® nuclease. In an aspect, an A gene promoter is operably linked to a nucleic acid encoding a guide nucleic acid. In an aspect, an A gene promoter is operably linked to a nucleic acid encoding a guide RNA. In an aspect, an A gene promoter is operably linked to a nucleic acid encoding a single-guide RNA.

In an aspect, a B gene promoter is operably linked to a nucleic acid encoding a guided nuclease. In an aspect, a B gene promoter is operably linked to a nucleic acid encoding a Cas12a nuclease. In an aspect, a B gene promoter is operably linked to a nucleic acid encoding a CasX nuclease. In an aspect, a B gene promoter is operably linked to a nucleic acid encoding a MAD7® nuclease. In an aspect, a B gene promoter is operably linked to a nucleic acid encoding a guide nucleic acid. In an aspect, a B gene promoter is operably linked to a nucleic acid encoding a guide RNA. In an aspect, a B gene promoter is operably linked to a nucleic acid encoding a single-guide RNA.

In an aspect, a C gene promoter is operably linked to a nucleic acid encoding a guided nuclease. In an aspect, a C gene promoter is operably linked to a nucleic acid encoding a Cas12a nuclease. In an aspect, a C gene promoter is operably linked to a nucleic acid encoding a CasX nuclease. In an aspect, a C gene promoter is operably linked to a nucleic acid encoding a MAD7® nuclease. In an aspect, a C gene promoter is operably linked to a nucleic acid encoding a guide nucleic acid. In an aspect, a C gene promoter is operably linked to a nucleic acid encoding a guide RNA. In an aspect, a C gene promoter is operably linked to a nucleic acid encoding a single-guide RNA.

In an aspect, a D gene promoter is operably linked to a nucleic acid encoding a guided nuclease. In an aspect, a D gene promoter is operably linked to a nucleic acid encoding a Cas12a nuclease. In an aspect, a D gene promoter is operably linked to a nucleic acid encoding a CasX nuclease. In an aspect, a D gene promoter is operably linked to a nucleic acid encoding a MAD7® nuclease. In an aspect, a D gene promoter is operably linked to a nucleic acid encoding a guide nucleic acid. In an aspect, a D gene promoter is operably linked to a nucleic acid encoding a guide RNA. In an aspect, a D gene promoter is operably linked to a nucleic acid encoding a single-guide RNA.

In an aspect, an E gene promoter is operably linked to a nucleic acid encoding a guided nuclease. In an aspect, an E gene promoter is operably linked to a nucleic acid encoding a Cas12a nuclease. In an aspect, an E gene promoter is operably linked to a nucleic acid encoding a CasX nuclease. In an aspect, an E gene promoter is operably linked to a nucleic acid encoding a MAD7® nuclease. In an aspect, an E gene promoter is operably linked to a nucleic acid encoding a guide nucleic acid. In an aspect, an E gene promoter is operably linked to a nucleic acid encoding a guide RNA. In an aspect, an E gene promoter is operably linked to a nucleic acid encoding a single-guide RNA.

In an aspect, an AP1 promoter is operably linked to a nucleic acid encoding a guided nuclease. In an aspect, an AP1 promoter is operably linked to a nucleic acid encoding a Cas12a nuclease. In an aspect, an AP1 promoter is operably linked to a nucleic acid encoding a CasX nuclease. In an aspect, an AP1 promoter is operably linked to a nucleic acid encoding a MAD7® nuclease. In an aspect, an AP1 promoter is operably linked to a nucleic acid encoding a guide nucleic acid. In an aspect, an AP1 promoter is operably linked to a nucleic acid encoding a guide RNA. In an aspect, an AP1 promoter is operably linked to a nucleic acid encoding a single-guide RNA.

In an aspect, an AP2 promoter is operably linked to a nucleic acid encoding a guided nuclease. In an aspect, an AP2 promoter is operably linked to a nucleic acid encoding a Cas12a nuclease. In an aspect, an AP2 promoter is operably linked to a nucleic acid encoding a CasX nuclease. In an aspect, an AP2 promoter is operably linked to a nucleic acid encoding a MAD7® nuclease. In an aspect, an AP2 promoter is operably linked to a nucleic acid encoding a guide nucleic acid. In an aspect, an AP2 promoter is operably linked to a nucleic acid encoding a guide RNA. In an aspect, an AP2 promoter is operably linked to a nucleic acid encoding a single-guide RNA.

In an aspect, a ZAP1 promoter is operably linked to a nucleic acid encoding a guided nuclease. In an aspect, a ZAP1 promoter is operably linked to a nucleic acid encoding a Cas12a nuclease. In an aspect, a ZAP1 promoter is operably linked to a nucleic acid encoding a CasX nuclease. In an aspect, a ZAP1 promoter is operably linked to a nucleic acid encoding a MAD7® nuclease. In an aspect, a ZAP1 promoter is operably linked to a nucleic acid encoding a guide nucleic acid. In an aspect, a ZAP1 promoter is operably linked to a nucleic acid encoding a guide RNA. In an aspect, a ZAP1 promoter is operably linked to a nucleic acid encoding a single-guide RNA.

In an aspect, an AP3 promoter is operably linked to a nucleic acid encoding a guided nuclease. In an aspect, an AP3 promoter is operably linked to a nucleic acid encoding a Cas12a nuclease. In an aspect, an AP3 promoter is operably linked to a nucleic acid encoding a CasX nuclease. In an aspect, an AP3 promoter is operably linked to a nucleic acid encoding a MAD7® nuclease. In an aspect, an AP3 promoter is operably linked to a nucleic acid encoding a guide nucleic acid. In an aspect, an AP3 promoter is operably linked to a nucleic acid encoding a guide RNA. In an aspect, an AP3 promoter is operably linked to a nucleic acid encoding a single-guide RNA.

In an aspect, a PI promoter is operably linked to a nucleic acid encoding a guided nuclease. In an aspect, a PI promoter is operably linked to a nucleic acid encoding a Cas12a nuclease. In an aspect, a PI promoter is operably linked to a nucleic acid encoding a CasX nuclease. In an aspect, a PI promoter is operably linked to a nucleic acid encoding a MAD7® nuclease. In an aspect, a PI promoter is operably linked to a nucleic acid encoding a guide nucleic acid. In an aspect, a PI promoter is operably linked to a nucleic acid encoding a guide RNA. In an aspect, a PI promoter is operably linked to a nucleic acid encoding a single-guide RNA.

In an aspect, a ZMM16 promoter is operably linked to a nucleic acid encoding a guided nuclease. In an aspect, a ZMM16 promoter is operably linked to a nucleic acid encoding a Cas12a nuclease. In an aspect, a ZMM16 promoter is operably linked to a nucleic acid encoding a CasX nuclease. In an aspect, a ZMM16 promoter is operably linked to a nucleic acid encoding a MAD7® nuclease. In an aspect, a ZMM16 promoter is operably linked to a nucleic acid encoding a guide nucleic acid. In an aspect, a ZMM16 promoter is operably linked to a nucleic acid encoding a guide RNA. In an aspect, a ZMM16 promoter is operably linked to a nucleic acid encoding a single-guide RNA.

In an aspect, a ZMM18 promoter is operably linked to a nucleic acid encoding a guided nuclease. In an aspect, a ZMM18 promoter is operably linked to a nucleic acid encoding a Cas12a nuclease. In an aspect, a ZMM18 promoter is operably linked to a nucleic acid encoding a CasX nuclease. In an aspect, a ZMM18 promoter is operably linked to a nucleic acid encoding a MAD7® nuclease. In an aspect, a ZMM18 promoter is operably linked to a nucleic acid encoding a guide nucleic acid. In an aspect, a ZMM18 promoter is operably linked to a nucleic acid encoding a guide RNA. In an aspect, a ZMM18 promoter is operably linked to a nucleic acid encoding a single-guide RNA.

In an aspect, an AP3 promoter is operably linked to a nucleic acid encoding a guided nuclease. In an aspect, an AP3 promoter is operably linked to a nucleic acid encoding a Cas12a nuclease. In an aspect, an AP3 promoter is operably linked to a nucleic acid encoding a CasX nuclease. In an aspect, an AP3 promoter is operably linked to a nucleic acid encoding a MAD7® nuclease. In an aspect, an AP3 promoter is operably linked to a nucleic acid encoding a guide nucleic acid. In an aspect, an AP3 promoter is operably linked to a nucleic acid encoding a guide RNA. In an aspect, an AP3 promoter is operably linked to a nucleic acid encoding a single-guide RNA.

In an aspect, an AG promoter is operably linked to a nucleic acid encoding a guided nuclease. In an aspect, an AG promoter is operably linked to a nucleic acid encoding a Cas12a nuclease. In an aspect, an AG promoter is operably linked to a nucleic acid encoding a CasX nuclease. In an aspect, an AG promoter is operably linked to a nucleic acid encoding a MAD7® nuclease. In an aspect, an AG promoter is operably linked to a nucleic acid encoding a guide nucleic acid. In an aspect, an AG promoter is operably linked to a nucleic acid encoding a guide RNA. In an aspect, an AG promoter is operably linked to a nucleic acid encoding a single-guide RNA.

In an aspect, a ZAG1 promoter is operably linked to a nucleic acid encoding a guided nuclease. In an aspect, a ZAG1 promoter is operably linked to a nucleic acid encoding a Cas12a nuclease. In an aspect, a ZAG1 promoter is operably linked to a nucleic acid encoding a CasX nuclease. In an aspect, a ZAG1 promoter is operably linked to a nucleic acid encoding a MAD7® nuclease. In an aspect, a ZAG1 promoter is operably linked to a nucleic acid encoding a guide nucleic acid. In an aspect, a ZAG1 promoter is operably linked to a nucleic acid encoding a guide RNA. In an aspect, a ZAG1 promoter is operably linked to a nucleic acid encoding a single-guide RNA.

In an aspect, a ZMM2 promoter is operably linked to a nucleic acid encoding a guided nuclease. In an aspect, a ZMM2 promoter is operably linked to a nucleic acid encoding a Cas12a nuclease. In an aspect, a ZMM2 promoter is operably linked to a nucleic acid encoding a CasX nuclease. In an aspect, a ZMM2 promoter is operably linked to a nucleic acid encoding a MAD7® nuclease. In an aspect, a ZMM2 promoter is operably linked to a nucleic acid encoding a guide nucleic acid. In an aspect, a ZMM2 promoter is operably linked to a nucleic acid encoding a guide RNA. In an aspect, a ZMM2 promoter is operably linked to a nucleic acid encoding a single-guide RNA.

In an aspect, a ZMM3 promoter is operably linked to a nucleic acid encoding a guided nuclease. In an aspect, a ZMM3 promoter is operably linked to a nucleic acid encoding a Cas12a nuclease. In an aspect, a ZMM3 promoter is operably linked to a nucleic acid encoding a CasX nuclease. In an aspect, a ZMM3 promoter is operably linked to a nucleic acid encoding a MAD7® nuclease. In an aspect, a ZMM3 promoter is operably linked to a nucleic acid encoding a guide nucleic acid. In an aspect, a ZMM3 promoter is operably linked to a nucleic acid encoding a guide RNA. In an aspect, a ZMM3 promoter is operably linked to a nucleic acid encoding a single-guide RNA.

In an aspect, an AGL11/STK promoter is operably linked to a nucleic acid encoding a guided nuclease. In an aspect, an AGL11/STK promoter is operably linked to a nucleic acid encoding a Cas12a nuclease. In an aspect, an AGL11/STK promoter is operably linked to a nucleic acid encoding a CasX nuclease. In an aspect, an AGL11/STK promoter is operably linked to a nucleic acid encoding a MAD7® nuclease. In an aspect, an AGL11/STK promoter is operably linked to a nucleic acid encoding a guide nucleic acid. In an aspect, an AGL11/STK promoter is operably linked to a nucleic acid encoding a guide RNA. In an aspect, an AGL11/STK promoter is operably linked to a nucleic acid encoding a single-guide RNA.

In an aspect, an AGL1/SHP1 promoter is operably linked to a nucleic acid encoding a guided nuclease. In an aspect, an AGL1/SHP1 promoter is operably linked to a nucleic acid encoding a Cas12a nuclease. In an aspect, an AGL1/SHP1 promoter is operably linked to a nucleic acid encoding a CasX nuclease. In an aspect, an AGL1/SHP1 promoter is operably linked to a nucleic acid encoding a MAD7® nuclease. In an aspect, an AGL1/SHP1 promoter is operably linked to a nucleic acid encoding a guide nucleic acid. In an aspect, an AGL1/SHP1 promoter is operably linked to a nucleic acid encoding a guide RNA. In an aspect, an AGL1/SHP1 promoter is operably linked to a nucleic acid encoding a single-guide RNA.

In an aspect, an AGL5/SHP2 promoter is operably linked to a nucleic acid encoding a guided nuclease. In an aspect, an AGL5/SHP2 promoter is operably linked to a nucleic acid encoding a Cas12a nuclease. In an aspect, an AGL5/SHP2 promoter is operably linked to a nucleic acid encoding a CasX nuclease. In an aspect, an AGL5/SHP2 promoter is operably linked to a nucleic acid encoding a MAD7® nuclease. In an aspect, an AGL5/SHP2 promoter is operably linked to a nucleic acid encoding a guide nucleic acid. In an aspect, an AGL5/SHP2 promoter is operably linked to a nucleic acid encoding a guide RNA. In an aspect, an AGL5/SHP2 promoter is operably linked to a nucleic acid encoding a single-guide RNA.

In an aspect, a ZAG2 promoter is operably linked to a nucleic acid encoding a guided nuclease. In an aspect, a ZAG2 promoter is operably linked to a nucleic acid encoding a Cas12a nuclease. In an aspect, a ZAG2 promoter is operably linked to a nucleic acid encoding a CasX nuclease. In an aspect, a ZAG2 promoter is operably linked to a nucleic acid encoding a MAD7® nuclease. In an aspect, a ZAG2 promoter is operably linked to a nucleic acid encoding a guide nucleic acid. In an aspect, a ZAG2 promoter is operably linked to a nucleic acid encoding a guide RNA. In an aspect, a ZAG2 promoter is operably linked to a nucleic acid encoding a single-guide RNA.

In an aspect, a ZMM1 promoter is operably linked to a nucleic acid encoding a guided nuclease. In an aspect, a ZMM1 promoter is operably linked to a nucleic acid encoding a Cas12a nuclease. In an aspect, a ZMM1 promoter is operably linked to a nucleic acid encoding a CasX nuclease. In an aspect, a ZMM1 promoter is operably linked to a nucleic acid encoding a MAD7® nuclease. In an aspect, a ZMM1 promoter is operably linked to a nucleic acid encoding a guide nucleic acid. In an aspect, a ZMM1 promoter is operably linked to a nucleic acid encoding a guide RNA. In an aspect, a ZMM1 promoter is operably linked to a nucleic acid encoding a single-guide RNA.

In an aspect, a SEP1 promoter is operably linked to a nucleic acid encoding a guided nuclease. In an aspect, a SEP1 promoter is operably linked to a nucleic acid encoding a Cas12a nuclease. In an aspect, a SEP1 promoter is operably linked to a nucleic acid encoding a CasX nuclease. In an aspect, a SEP1 promoter is operably linked to a nucleic acid encoding a MAD7® nuclease. In an aspect, a SEP1 promoter is operably linked to a nucleic acid encoding a guide nucleic acid. In an aspect, a SEP1 promoter is operably linked to a nucleic acid encoding a guide RNA. In an aspect, a SEP1 promoter is operably linked to a nucleic acid encoding a single-guide RNA.

In an aspect, a SEP2 promoter is operably linked to a nucleic acid encoding a guided nuclease. In an aspect, a SEP2 promoter is operably linked to a nucleic acid encoding a Cas12a nuclease. In an aspect, a SEP2 promoter is operably linked to a nucleic acid encoding a CasX nuclease. In an aspect, a SEP2 promoter is operably linked to a nucleic acid encoding a MAD7® nuclease. In an aspect, a SEP2 promoter is operably linked to a nucleic acid encoding a guide nucleic acid. In an aspect, a SEP2 promoter is operably linked to a nucleic acid encoding a guide RNA. In an aspect, a SEP2 promoter is operably linked to a nucleic acid encoding a single-guide RNA.

In an aspect, a SEP3 promoter is operably linked to a nucleic acid encoding a guided nuclease. In an aspect, a SEP3 promoter is operably linked to a nucleic acid encoding a Cas12a nuclease. In an aspect, a SEP3 promoter is operably linked to a nucleic acid encoding a CasX nuclease. In an aspect, a SEP3 promoter is operably linked to a nucleic acid encoding a MAD7® nuclease. In an aspect, a SEP3 promoter is operably linked to a nucleic acid encoding a guide nucleic acid. In an aspect, a SEP3 promoter is operably linked to a nucleic acid encoding a guide RNA. In an aspect, a SEP3 promoter is operably linked to a nucleic acid encoding a single-guide RNA.

In an aspect, a SEP4 promoter is operably linked to a nucleic acid encoding a guided nuclease. In an aspect, a SEP4 promoter is operably linked to a nucleic acid encoding a Cas12a nuclease. In an aspect, a SEP4 promoter is operably linked to a nucleic acid encoding a CasX nuclease. In an aspect, a SEP4 promoter is operably linked to a nucleic acid encoding a MAD7® nuclease. In an aspect, a SEP4 promoter is operably linked to a nucleic acid encoding a guide nucleic acid. In an aspect, a SEP4 promoter is operably linked to a nucleic acid encoding a guide RNA. In an aspect, a SEP4 promoter is operably linked to a nucleic acid encoding a single-guide RNA.

In an aspect, a ZAG3 promoter is operably linked to a nucleic acid encoding a guided nuclease. In an aspect, a ZAG3 promoter is operably linked to a nucleic acid encoding a Cas12a nuclease. In an aspect, a ZAG3 promoter is operably linked to a nucleic acid encoding a CasX nuclease. In an aspect, a ZAG3 promoter is operably linked to a nucleic acid encoding a MAD7® nuclease. In an aspect, a ZAG3 promoter is operably linked to a nucleic acid encoding a guide nucleic acid. In an aspect, a ZAG3 promoter is operably linked to a nucleic acid encoding a guide RNA. In an aspect, a ZAG3 promoter is operably linked to a nucleic acid encoding a single-guide RNA.

In an aspect, a ZMM7/SEP-like promoter is operably linked to a nucleic acid encoding a guided nuclease. In an aspect, a ZMM7/SEP-like promoter is operably linked to a nucleic acid encoding a Cas12a nuclease. In an aspect, a ZMM7/SEP-like promoter is operably linked to a nucleic acid encoding a CasX nuclease. In an aspect, a ZMM7 promoter is operably linked to a nucleic acid encoding a MAD7® nuclease. In an aspect, a ZMM7/SEP-like promoter is operably linked to a nucleic acid encoding a guide nucleic acid. In an aspect, a ZMM7/SEP-like promoter is operably linked to a nucleic acid encoding a guide RNA. In an aspect, a ZMM7/SEP-like promoter is operably linked to a nucleic acid encoding a single-guide RNA.

In an aspect, a promoter selected from the group consisting of SEQ ID NOs: 1-30, or a promoter selected from the group of SEQ ID NOs: 1-16, 18-19, 21-30 and 45-49, or a functional fragment thereof, is operably linked to a nucleic acid encoding a guided nuclease. In an aspect, a promoter selected from the group consisting of SEQ ID NOs: 1-30, or a promoter selected from the group of SEQ ID NOs: 1-16, 18-19, 21-30 and 45-49, or a functional fragment thereof, is operably linked to a nucleic acid encoding a Cas12a nuclease. In an aspect, a promoter selected from the group consisting of SEQ ID NOs: 1-30, or a promoter selected from the group of SEQ ID NOs: 1-16, 18-19, 21-30 and 45-49, or a functional fragment thereof, is operably linked to a nucleic acid encoding a CasX nuclease. In an aspect, a promoter selected from the group consisting of SEQ ID NOs: 1-30, or a promoter selected from the group of SEQ ID NOs: 1-16, 18-19, 21-30 and 45-49, or a functional fragment thereof, is operably linked to a nucleic acid encoding a MAD7® nuclease. In an aspect, a promoter selected from the group consisting of SEQ ID NOs: 1-30, or a promoter selected from the group of SEQ ID NOs: 1-16, 18-19, 21-30 and 45-49, or a functional fragment thereof, is operably linked to a nucleic acid encoding a guide nucleic acid. In an aspect, a promoter selected from the group consisting of SEQ ID NOs: 1-30, or a promoter selected from the group of SEQ ID NOs: 1-16, 18-19, 21-30 and 45-49, or a functional fragment thereof, is operably linked to a nucleic acid encoding a guide RNA. In an aspect, a promoter selected from the group consisting of SEQ ID NOs: 1-30, or a promoter selected from the group of SEQ ID NOs: 1-16, 18-19, 21-30 and 45-49, or a functional fragment thereof, is operably linked to a nucleic acid encoding a single-guide RNA.

As used herein, a “floral tissue-preferred promoter” refers to a promoter that exhibits higher, or preferential, expression in floral tissue as compared to other cell or tissue types of a plant. Floral tissue-preferred promoters can exhibit expression in any floral tissue, as well as nearby cells or tissues, such as, without being limiting, stem cells, vascular cells, and trichome cells. A floral tissue-preferred promoter can also exhibit expression in other plant tissues or cells, such as, without being limiting, root cells, egg cells, endosperm cells, cotyledon cells, seed coat cells, leaf cells, vascular cells, embryo cells, and shoot apical meristem cells.

As used herein, a “floral tissue-specific promoter” refers to a promoter that exhibits expression exclusively in floral tissues. In an aspect, a floral tissue-preferred promoter comprises a floral tissue-specific promoter.

As used herein, a “floral cell-preferred promoter” refers to a promoter that exhibits higher, or preferential, expression in floral cells as compared to other cell or tissue types of a plant. Floral cell-preferred promoters can exhibit expression in any floral cell, as well as nearby cells or tissues, such as, without being limiting, stem cells, vascular cells, and trichome cells. A floral cell-preferred promoter can also exhibit expression in other plant tissues or cells, such as, without being limiting, root cells, egg cells, endosperm cells, cotyledon cells, seed coat cells, leaf cells, vascular cells, embryo cells, and shoot apical meristem cells.

As used herein, a “floral cell-specific promoter” refers to a promoter that exhibits expression exclusively in floral cells. In an aspect, a floral cell-preferred promoter comprises a floral cell-specific promoter.

It is appreciated in the art that a fragment of a promoter sequence can function to drive transcription of an operably linked nucleic acid molecule. For example, without being limiting, if a 1000 nucleotide promoter is truncated to 500 nucleotide, and the 500 nucleotide fragment is capable of driving transcription, the 500 nucleotide fragment is referred to as a “functional fragment.” It is appreciated in the art that a promoter can a variant. As used herein, the term “variant” refers to a second DNA molecule, such as a regulatory element, that is in composition similar, but not identical to, a first DNA molecule, and wherein the second DNA molecule still maintains the general functionality, i.e. the same or similar expression pattern, for instance through more or less equivalent transcriptional activity, of the first DNA molecule. A variant may be a shorter, longer or truncated version of the first DNA molecule or an altered version of the sequence of the first DNA molecule, such as one with different restriction enzyme sites and/or internal deletions, substitutions, or insertions. A “variant” can also encompass a regulatory element having a nucleotide sequence comprising a substitution, deletion, or insertion of one or more nucleotides of a reference sequence, wherein the derivative regulatory element has more or less or equivalent transcriptional or translational activity than the corresponding parent regulatory molecule.

Promoters that drive expression in all or most tissues of the plant are referred to as “constitutive” promoters. Promoters that drive expression during certain periods or stages of development are referred to as “developmental” promoters. An “inducible” promoter is a promoter that initiates transcription in response to an environmental stimulus such as heat, cold, drought, light, or other stimuli, such as wounding or chemical application. A promoter can also be classified in terms of its origin, such as being heterologous, homologous, chimeric, synthetic, etc.

As used herein, the term “heterologous” in reference to a promoter is a promoter sequence having a different origin relative to its associated transcribable DNA sequence, coding sequence or gene (or transgene), and/or not naturally occurring in the plant species to be transformed. The term “heterologous” can refer more broadly to a combination of two or more DNA molecules or sequences, such as a promoter and an associated transcribable DNA sequence, coding sequence or gene, when such a combination is man-made and not normally found in nature.

In an aspect, a promoter provided herein is a constitutive promoter. In still another aspect, a promoter provided herein is an inducible promoter. In an aspect, a promoter provided herein is selected from the group consisting of a constitutive promoter, a tissue-specific promoter, a tissue-preferred promoter, and an inducible promoter.

RNA polymerase III (Pol III) promoters can be used to drive the expression of non-protein coding RNA molecules. In an aspect, a promoter provided herein is a Pol III promoter. In another aspect, a Pol III promoter provided herein is operably linked to a nucleic acid molecule encoding a non-protein coding RNA. In yet another aspect, a Pol III promoter provided herein is operably linked to a nucleic acid molecule encoding a guide nucleic acid. In still another aspect, a Pol III promoter provided herein is operably linked to a nucleic acid molecule encoding a single-guide RNA. In a further aspect, a Pol III promoter provided herein is operably linked to a nucleic acid molecule encoding a CRISPR RNA (crRNA). In another aspect, a Pol III promoter provided herein is operably linked to a nucleic acid molecule encoding a tracer RNA (tracrRNA).

Non-limiting examples of Pol III promoters include a U6 promoter, an H1 promoter, a 5S promoter, an Adenovirus 2 (Ad2) VAI promoter, a tRNA promoter, and a 7SK promoter. See, for example, Schramm and Hernandez, 2002, Genes & Development, 16:2593-2620, which is incorporated by reference herein in its entirety. In an aspect, a Pol III promoter provided herein is selected from the group consisting of a U6 promoter, an H1 promoter, a 5S promoter, an Adenovirus 2 (Ad2) VAI promoter, a tRNA promoter, and a 7SK promoter. In another aspect, a guide RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a single-guide RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a CRISPR RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a tracer RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter.

In an aspect, a promoter provided herein is a Dahlia Mosaic Virus (DaMV) promoter. In another aspect, a promoter provided herein is a U6 promoter. In another aspect, a promoter provided herein is an actin promoter. In an aspect, a promoter provided herein is a Cauliflower Mosaic Virus (CaMV) 35S promoter. In an aspect, a promoter provided herein is a ubiquitin promoter.

In an aspect, a constitutive promoter is selected from the group consisting of a CaMV 35S promoter, an actin promoter, and a ubiquitin promoter.

Examples describing a promoter that can be used herein include without limitation U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No. 6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter), and U.S. patent application Ser. No. 09/757,089 (maize chloroplast aldolase promoter). Additional promoters that can find use are a nopaline synthase (NOS) promoter (Ebert et al., 1987), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al., Plant Molecular Biology (1987) 9: 315-324), the CaMV 35S promoter (Odell et al., Nature (1985) 313: 810-812), the figwort mosaic virus 35S-promoter (U.S. Pat. Nos. 6,051,753; 5,378,619), the sucrose synthase promoter (Yang and Russell, Proceedings of the National Academy of Sciences, USA (1990) 87: 4144-4148), the R gene complex promoter (Chandler et al., Plant Cell (1989) 1: 1175-1183), and the chlorophyll a/b binding protein gene promoter, PC1SV (U.S. Pat. No. 5,850,019), and AGRtu.nos (GenBank Accession V00087; Depicker et al., Journal of Molecular and Applied Genetics (1982) 1: 561-573; Bevan et al., 1983) promoters.

Promoter hybrids can also be used and constructed to enhance transcriptional activity (see U.S. Pat. No. 5,106,739), or to combine desired transcriptional activity, inducibility and tissue specificity or developmental specificity. Promoters that function in plants include but are not limited to promoters that are inducible, viral, synthetic, constitutive, temporally regulated, spatially regulated, and spatio-temporally regulated. Other promoters that are tissue-enhanced, tissue-specific, or developmentally regulated are also known in the art and envisioned to have utility in the practice of this disclosure.

In an aspect, a constitutive promoter is operably linked to a nucleic acid sequence encoding a guided nuclease. In an aspect, a constitutive promoter is operably linked to a nucleic acid sequence encoding a Cas12a nuclease. In an aspect, a constitutive promoter is operably linked to a nucleic acid sequence encoding a CasX nuclease. In an aspect, a constitutive promoter is operably linked to a nucleic acid sequence encoding a MAD7® nuclease. In an aspect, a constitutive promoter is operably linked to a nucleic acid sequence encoding a guide nucleic acid. In an aspect, a constitutive promoter is operably linked to a nucleic acid sequence encoding a guide RNA. In an aspect, a constitutive promoter is operably linked to a nucleic acid sequence encoding a single-guide RNA.

In an aspect, an inducible promoter is operably linked to a nucleic acid sequence encoding a guided nuclease. In an aspect, an inducible promoter is operably linked to a nucleic acid sequence encoding a Cas12a nuclease. In an aspect, an inducible promoter is operably linked to a nucleic acid sequence encoding a CasX nuclease. In an aspect, an inducible promoter is operably linked to a nucleic acid sequence encoding a MAD7® nuclease. In an aspect, an inducible promoter is operably linked to a nucleic acid sequence encoding a guide nucleic acid. In an aspect, an inducible promoter is operably linked to a nucleic acid sequence encoding a guide RNA. In an aspect, an inducible promoter is operably linked to a nucleic acid sequence encoding a single-guide RNA.

In an aspect, a developmental promoter is operably linked to a nucleic acid sequence encoding a guided nuclease. In an aspect, a developmental promoter is operably linked to a nucleic acid sequence encoding a Cas12a nuclease. In an aspect, a developmental promoter is operably linked to a nucleic acid sequence encoding a CasX nuclease. In an aspect, a developmental promoter is operably linked to a nucleic acid sequence encoding a MAD7® nuclease. In an aspect, a developmental promoter is operably linked to a nucleic acid sequence encoding a guide nucleic acid. In an aspect, a developmental promoter is operably linked to a nucleic acid sequence encoding a guide RNA. In an aspect, a developmental promoter is operably linked to a nucleic acid sequence encoding a single-guide RNA.

### Transcription Activator-Like Effectors (TALEs)

Transcription Activator-Like Effectors (TALEs) are transcription factors that comprise a C terminal activation domain and can activate/increase the expression of an operably linked transcribable polynucleotide once TALEs bind to the TALE binding site at or near the promoter. Without being limited by any theory, it has previously been shown that TALE proteins can induce high expression of a gene operably linked to a TALE binding site, and that expression can be modulated depending on how many of the TALE binding sites are present in the regulatory region. In an aspect, a promoter selected from the group consisting of SEQ ID NOs: 1-30, or selected from the group consisting of SEQ ID NOs: 1-16, 18-19, 21-30 and 45-49, or a functional fragment or variant thereof, is operably linked to a nucleic acid encoding a TALE.

In an aspect, a TALE binding site is operably linked to a promoter. In an aspect, at least two TALE binding sites are operably linked to a promoter. In an aspect, at least three TALE binding sites are operably linked to a promoter. In an aspect, at least four TALE binding sites are operably linked to a promoter. In an aspect, at least five TALE binding sites are operably linked to a promoter. In an aspect, at least six TALE binding sites are operably linked to a promoter. In an aspect, at least seven TALE binding sites are operably linked to a promoter. In an aspect, at least eight TALE binding sites are operably linked to a promoter. In an aspect, at least nine TALE binding sites are operably linked to a promoter. In an aspect, at least ten TALE binding sites are operably linked to a promoter.

### Guided Nucleases

Guided nucleases are nucleases that form a complex (e.g., a ribonucleoprotein) with a guide nucleic acid molecule (e.g., a guide RNA), which then guides the complex to a target site within a target sequence. One non-limiting example of guided nucleases are CRISPR nucleases.

CRISPR (Clustered Regularly Interspaced Short Palindromic Repeats) nucleases (e.g. Cas9, CasX, Cas12a (also referred to as Cpf1), CasY, MAD7®) are proteins found in bacteria that are guided by guide RNAs (“gRNAs”) to a target nucleic acid molecule, where the endonuclease can then cleave one or two strands the target nucleic acid molecule. Although the origins of CRISPR nucleases are bacterial, many CRISPR nucleases have been shown to function in eukaryotic cells.

While not being limited by any particular scientific theory, a CRISPR nuclease forms a complex with a guide RNA (gRNA), which hybridizes with a complementary target site, thereby guiding the CRISPR nuclease to the target site. In class II CRISPR-Cas systems, CRISPR arrays, including spacers, are transcribed during encounters with recognized invasive DNA and are processed into small interfering CRISPR RNAs (crRNAs). The crRNA comprises a repeat sequence and a spacer sequence which is complementary to a specific protospacer sequence in an invading pathogen. The spacer sequence can be designed to be complementary to target sequences in a eukaryotic genome.

CRISPR nucleases associate with their respective crRNAs in their active forms. CasX, similar to the class II endonuclease Cas9, requires another non-coding RNA component, referred to as a trans-activating crRNA (tracrRNA), to have functional activity. Nucleic acid molecules provided herein can combine a crRNA and a tracrRNA into one nucleic acid molecule in what is herein referred to as a “single guide RNA” (sgRNA). Cas12a or MAD7® do not require a tracrRNA to be guided to a target site; a crRNA alone is sufficient for Cas12a or MAD7®. The gRNA guides the active CRISPR nuclease complex to a target site, where the CRISPR nuclease can cleave the target site.

When an RNA-guided CRISPR nuclease and a guide RNA form a complex, the whole system is called a “ribonucleoprotein.” Ribonucleoproteins provided herein can also comprise additional nucleic acids or proteins.

In an aspect, a guided nuclease and a guide nucleic acid form a ribonucleoprotein in a floral cell. In another aspect, a guided nuclease and a guide nucleic acid form a ribonucleoprotein in a floral tissue. In an aspect, a Cas12a nuclease and a guide nucleic acid form a ribonucleoprotein in a floral cell. In another aspect, a Cas12a nuclease and a guide nucleic acid form a ribonucleoprotein in a floral tissue. In an aspect, a CasX nuclease and a guide nucleic acid form a ribonucleoprotein in a floral cell. In another aspect, a CasX nuclease and a guide nucleic acid form a ribonucleoprotein in a floral tissue. In an aspect, a MAD7® nuclease and a guide nucleic acid form a ribonucleoprotein in a floral cell. In another aspect, a MAD7® nuclease and a guide nucleic acid form a ribonucleoprotein in a floral tissue. In an aspect, a guided nuclease and a guide RNA form a ribonucleoprotein in a floral cell. In another aspect, a guided nuclease and a guide RNA form a ribonucleoprotein in a floral tissue. In an aspect, a Cas12a nuclease and a guide RNA form a ribonucleoprotein in a floral cell. In another aspect, a CasX nuclease and a guide RNA form a ribonucleoprotein in a floral tissue. In an aspect, a guided nuclease and a single-guide RNA form a ribonucleoprotein in a floral cell. In another aspect, a guided nuclease and a single-guide RNA form a ribonucleoprotein in a floral tissue. In another aspect, a CasX nuclease and a single-guide RNA form a ribonucleoprotein in a floral tissue. In another aspect, a MAD7® nuclease and a single-guide RNA form a ribonucleoprotein in a floral tissue.

In an aspect, a ribonucleoprotein generates at least one double-stranded break within a target site in a floral cell. In an aspect, a ribonucleoprotein generates at least one double-stranded break within a target site in floral tissue. In an aspect, a ribonucleoprotein generates at least one single-stranded break within a target site in a floral cell. In an aspect, a ribonucleoprotein generates at least one single-stranded break within a target site in floral tissue.

A prerequisite for cleavage of the target site by a CRISPR ribonucleoprotein is the presence of a conserved Protospacer Adjacent Motif (PAM) near the target site. Depending on the CRISPR nuclease, cleavage can occur within a certain number of nucleotides (e.g., between 18-23 nucleotides for Cas12a) from the PAM site. PAM sites are only required for type I and type II CRISPR associated proteins, and different CRISPR endonucleases recognize different PAM sites. Without being limiting, Cas12a can recognize at least the following PAM sites: TTTN, and YTN; CasX can recognize at least the following PAM sites: TTCN, TTCA, and TTC and MAD7® nuclease recognizes T-rich PAM sequences YTTN and seems to prefer TTTN to CTTN PAMs (where T is thymine; C is cytosine; A is adenine; Y is thymine or cytosine; and N is thymine, cytosine, guanine, or adenine).

Cas12a is an RNA-guided nuclease of a class II, type V CRISPR/Cas system. Cas12a nucleases generate staggered cuts when cleaving a double-stranded DNA molecule. Staggered cuts of double-stranded DNA produce a single-stranded DNA overhang of at least one nucleotide. This is in contrast to a blunt-end cut (such as those generated by Cas9), which does not produce a single-stranded DNA overhang when cutting double-stranded DNA.

In an aspect, a Cas12a nuclease provided herein is a Lachnospiraceae bacterium Cas12a (LbCas12a) nuclease. In another aspect, a Cas12a nuclease provided herein is a Francisella novicida Cas12a (FnCas12a) nuclease. In an aspect, a Cas12a nuclease is selected from the group consisting of LbCas12a and FnCas12a.

In an aspect, a Cas12a nuclease, or a nucleic acid encoding a Cas12a nuclease, is derived from a bacteria genus selected from the group consisting of Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Acidaminococcus, Peregrinibacteria, Butyrivibrio, Parcubacteria, Smithella, Candidatus, Moraxella, and Leptospira.

In an aspect, a Cas12a nuclease is encoded by a polynucleotide comprising a sequence at least 80% identical to a polynucleotide selected from the group consisting of SEQ ID NO: 32 and SEQ ID NO: 36. In another aspect, a Cas12a nuclease is encoded by a polynucleotide comprising a sequence at least 85% identical to a polynucleotide selected from the group consisting of SEQ ID NO: 32 and SEQ ID NO: 36. In another aspect, a Cas12a nuclease is encoded by a polynucleotide comprising a sequence at least 90% identical to a polynucleotide selected from the group consisting of SEQ ID NO: 32 and SEQ ID NO: 36. In another aspect, a Cas12a nuclease is encoded by a polynucleotide comprising a sequence at least 95% identical to a polynucleotide selected from the group consisting of SEQ ID NO: 32 and SEQ ID NO: 36. In another aspect, a Cas12a nuclease is encoded by a polynucleotide comprising a sequence at least 96% identical to a polynucleotide selected from the group consisting of SEQ ID NO: 32 and SEQ ID NO: 36. In another aspect, a Cas12a nuclease is encoded by a polynucleotide comprising a sequence at least 97% identical to a polynucleotide selected from the group consisting of SEQ ID NO: 32 and SEQ ID NO: 36. In another aspect, a Cas12a nuclease is encoded by a polynucleotide comprising a sequence at least 98% identical to a polynucleotide selected from the group consisting of SEQ ID NO: 32 and SEQ ID NO: 36. In another aspect, a Cas12a nuclease is encoded by a polynucleotide comprising a sequence at least 99% identical to a polynucleotide selected from the group consisting of SEQ ID NO: 32 and SEQ ID NO: 36. In another aspect, a Cas12a nuclease is encoded by a polynucleotide comprising a sequence 100% identical to a polynucleotide selected from the group consisting of SEQ ID NO: 32 and SEQ ID NO: 36.

CasX is a type of class II CRISPR-Cas nuclease that has been identified in the bacterial phyla Deltaproteobacteria and Planctomycetes. Similar to Cas12a, CasX nucleases generate staggered cuts when cleaving a double-stranded DNA molecule. However, unlike Cas12a, CasX nucleases require a crRNA and a tracrRNA, or a single-guide RNA, in order to target and cleave a target nucleic acid.

In an aspect, a CasX nuclease provided herein is a CasX nuclease from the phylum Deltaproteobacteria. In another aspect, a CasX nuclease provided herein is a CasX nuclease from the phylum Planctomycetes. Without being limiting, additional suitable CasX nucleases are those set forth in WO 2019/084148, which is incorporated by reference herein in its entirety.

MAD7® (also known as ErCas12a) is an engineered nuclease of the Class 2 type V-A CRISPR-Cas (Cas12a/Cpf1) family with a low level of homology to canonical Cas12a nucleases. MAD7® nucleases generate staggered cuts when cleaving a double-stranded DNA molecule. MAD7® nuclease was initially identified in Eubacterium rectale. It only requires a crRNA like canonical Cas12a. An ErCas12a/MAD7® encoding nucleotide sequence can be found in the supplementary data (sequences S1) provided with Lin et al., Journal of Genetics and Genomics, 48:444-451 (2021).

In an aspect, a guided nuclease capable of generating a staggered cut in a double-stranded DNA molecule is selected from the group consisting of Cas12a; MAD7® and CasX. In an aspect, a guided nuclease is selected from the group consisting of Cas12a, MAD7® and CasX.

In an aspect, a guided nuclease is a RNA-guided nuclease. In another aspect, a guided nuclease is a CRISPR nuclease, In another aspect, a guided nuclease is a Cas12a nuclease. In another aspect, a guided nuclease is a CasX nuclease. In another aspect, a guided nuclease is a MAD7® nuclease.

As used herein, a “nuclear localization signal” (NLS) refers to an amino acid sequence that “tags” a protein for import into the nucleus of a cell. In an aspect, a nucleic acid molecule provided herein encodes a nuclear localization signal. In another aspect, a nucleic acid molecule provided herein encodes two or more nuclear localization signals.

In an aspect, a Cas12a nuclease provided herein comprises a nuclear localization signal. In an aspect, a nuclear localization signal is positioned on the N-terminal end of a Cas12a nuclease. In a further aspect, a nuclear localization signal is positioned on the C-terminal end of a Cas12a nuclease. In yet another aspect, a nuclear localization signal is positioned on both the N-terminal end and the C-terminal end of a Cas12a nuclease.

In an aspect, a CasX nuclease provided herein comprises a nuclear localization signal. In an aspect, a nuclear localization signal is positioned on the N-terminal end of a CasX nuclease. In a further aspect, a nuclear localization signal is positioned on the C-terminal end of a CasX nuclease. In yet another aspect, a nuclear localization signal is positioned on both the N-terminal end and the C-terminal end of a CasX nuclease.

In an aspect, a MAD7® nuclease provided herein comprises a nuclear localization signal. In an aspect, a nuclear localization signal is positioned on the N-terminal end of a MAD7® nuclease. In a further aspect, a nuclear localization signal is positioned on the C-terminal end of a MAD7® nuclease. In yet another aspect, a nuclear localization signal is positioned on both the N-terminal end and the C-terminal end of a MAD7® nuclease

In an aspect, a ribonucleoprotein comprises at least one nuclear localization signal. In another aspect, a ribonucleoprotein comprises at least two nuclear localization signals.

In an aspect, a nuclear localization signal provided herein is encoded by SEQ ID NO: 33 or 34.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www[dot]kazusa[dot]or[dot]jp[forwards slash]codon and these tables can be adapted in a number of ways. See Nakamura et al., 2000, Nucl. Acids Res. 28:292. Computer algorithms for codon optimizing a particular sequence for expression in a particular plant cell are also available, such as Gene Forge (Aptagen; Jacobus, PA), are also available.

As used herein, “codon optimization” refers to a process of modifying a nucleic acid sequence for enhanced expression in a plant cell of interest by replacing at least one codon (e.g., at least 1, 2, 3, 4, 5, 10, 15, 20, 25, 50, or more codons) of a sequence with codons that are more frequently or most frequently used in the genes of the plant cell while maintaining the original amino acid sequence (e.g., introducing silent mutations).

In an aspect, one or more codons (e.g., 1, 2, 3, 4, 5, 10, 15, 20, 25, 50, or more, or all codons) in a sequence encoding a guided nuclease correspond to the most frequently used codon for a particular amino acid. In another aspect, one or more codons (e.g., 1, 2, 3, 4, 5, 10, 15, 20, 25, 50, or more, or all codons) in a sequence encoding a Cas12a nuclease or a CasX nuclease or a MAD7® nuclease correspond to the most frequently used codon for a particular amino acid. As to codon usage in plants, reference is made to Campbell and Gowri, 1990, Plant Physiol., 92: 1-11; and Murray et al., 1989, Nucleic Acids Res., 17:477-98, each of which is incorporated herein by reference in their entireties.

In an aspect, a nucleic acid molecule encodes a guided nuclease that is codon optimized for a plant. In an aspect, a nucleic acid molecule encodes a Cas12a nuclease that is codon optimized for a plant. In an aspect, a nucleic acid molecule encodes a CasX nuclease that is codon optimized for a plant. In an aspect, a nucleic acid molecule encodes a MAD7® nuclease that is codon optimized for a plant

In another aspect, a nucleic acid molecule provided herein encodes a guided nuclease that is codon optimized for a plant cell. In another aspect, a nucleic acid molecule provided herein encodes a guided nuclease that is codon optimized for a monocotyledonous plant species. In another aspect, a nucleic acid molecule provided herein encodes a guided nuclease that is codon optimized for a dicotyledonous plant species. In a further aspect, a nucleic acid molecule provided herein encodes a guided nuclease that is codon optimized for a gymnosperm plant species. In a further aspect, a nucleic acid molecule provided herein encodes a guided nuclease that is codon optimized for an angiosperm plant species. In a further aspect, a nucleic acid molecule provided herein encodes a guided nuclease that is codon optimized for a corn cell. In a further aspect, a nucleic acid molecule provided herein encodes a guided nuclease that is codon optimized for a soybean cell. In a further aspect, a nucleic acid molecule provided herein encodes a guided nuclease that is codon optimized for a rice cell. In a further aspect, a nucleic acid molecule provided herein encodes a guided nuclease that is codon optimized for a wheat cell. In a further aspect, a nucleic acid molecule provided herein encodes a guided nuclease that is codon optimized for a cotton cell. In a further aspect, a nucleic acid molecule provided herein encodes a guided nuclease that is codon optimized for a sorghum cell. In a further aspect, a nucleic acid molecule provided herein encodes a guided nuclease that is codon optimized for an alfalfa cell. In a further aspect, a nucleic acid molecule provided herein encodes a guided nuclease that is codon optimized for a sugarcane cell. In a further aspect, a nucleic acid molecule provided herein encodes a guided nuclease that is codon optimized for an Arabidopsis cell. In a further aspect, a nucleic acid molecule provided herein encodes a guided nuclease that is codon optimized for a tomato cell. In a further aspect, a nucleic acid molecule provided herein encodes a guided nuclease that is codon optimized for a cucumber cell. In a further aspect, a nucleic acid molecule provided herein encodes a guided nuclease that is codon optimized for a potato cell. In a further aspect, a nucleic acid molecule provided herein encodes a guided nuclease that is codon optimized for an onion cell.

In another aspect, a nucleic acid molecule provided herein encodes a Cas12a nuclease that is codon optimized for a plant cell. In another aspect, a nucleic acid molecule provided herein encodes a Cas12a nuclease that is codon optimized for a monocotyledonous plant species. In another aspect, a nucleic acid molecule provided herein encodes a Cas12a nuclease that is codon optimized for a dicotyledonous plant species. In a further aspect, a nucleic acid molecule provided herein encodes a Cas12a nuclease that is codon optimized for a gymnosperm plant species. In a further aspect, a nucleic acid molecule provided herein encodes a Cas12a nuclease that is codon optimized for an angiosperm plant species. In a further aspect, a nucleic acid molecule provided herein encodes a Cas12a nuclease that is codon optimized for a corn cell. In a further aspect, a nucleic acid molecule provided herein encodes a Cas12a nuclease that is codon optimized for a soybean cell. In a further aspect, a nucleic acid molecule provided herein encodes a Cas12a nuclease that is codon optimized for a rice cell. In a further aspect, a nucleic acid molecule provided herein encodes a Cas12a nuclease that is codon optimized for a wheat cell. In a further aspect, a nucleic acid molecule provided herein encodes a Cas12a nuclease that is codon optimized for a cotton cell. In a further aspect, a nucleic acid molecule provided herein encodes a Cas12a nuclease that is codon optimized for a sorghum cell. In a further aspect, a nucleic acid molecule provided herein encodes a Cas12a nuclease that is codon optimized for an alfalfa cell. In a further aspect, a nucleic acid molecule provided herein encodes a Cas12a nuclease that is codon optimized for a sugar cane cell. In a further aspect, a nucleic acid molecule provided herein encodes a Cas12a nuclease that is codon optimized for an Arabidopsis cell. In a further aspect, a nucleic acid molecule provided herein encodes a Cas12a nuclease that is codon optimized for a tomato cell. In a further aspect, a nucleic acid molecule provided herein encodes a Cas12a nuclease that is codon optimized for a cucumber cell. In a further aspect, a nucleic acid molecule provided herein encodes a Cas12a nuclease that is codon optimized for a potato cell. In a further aspect, a nucleic acid molecule provided herein encodes a Cas12a nuclease that is codon optimized for an onion cell. In another aspect, a nucleic acid molecule provided herein encodes a CasX nuclease that is codon optimized for a plant cell. In another aspect, a nucleic acid molecule provided herein encodes a CasX nuclease that is codon optimized for a monocotyledonous plant species. In another aspect, a nucleic acid molecule provided herein encodes a CasX nuclease that is codon optimized for a dicotyledonous plant species. In a further aspect, a nucleic acid molecule provided herein encodes a CasX nuclease that is codon optimized for a gymnosperm plant species. In a further aspect, a nucleic acid molecule provided herein encodes a CasX nuclease that is codon optimized for an angiosperm plant species. In a further aspect, a nucleic acid molecule provided herein encodes a CasX nuclease that is codon optimized for a corn cell. In a further aspect, a nucleic acid molecule provided herein encodes a CasX nuclease that is codon optimized for a soybean cell. In a further aspect, a nucleic acid molecule provided herein encodes a CasX nuclease that is codon optimized for a rice cell. In a further aspect, a nucleic acid molecule provided herein encodes a CasX nuclease that is codon optimized for a wheat cell. In a further aspect, a nucleic acid molecule provided herein encodes a CasX nuclease that is codon optimized for a cotton cell. In a further aspect, a nucleic acid molecule provided herein encodes a CasX nuclease that is codon optimized for a sorghum cell. In a further aspect, a nucleic acid molecule provided herein encodes a CasX nuclease that is codon optimized for an alfalfa cell. In a further aspect, a nucleic acid molecule provided herein encodes a CasX nuclease that is codon optimized for a sugar cane cell. In a further aspect, a nucleic acid molecule provided herein encodes a CasX nuclease that is codon optimized for an Arabidopsis cell. In a further aspect, a nucleic acid molecule provided herein encodes a CasX nuclease that is codon optimized for a tomato cell. In a further aspect, a nucleic acid molecule provided herein encodes a CasX nuclease that is codon optimized for a cucumber cell. In a further aspect, a nucleic acid molecule provided herein encodes a CasX nuclease that is codon optimized for a potato cell. In a further aspect, a nucleic acid molecule provided herein encodes a CasX nuclease that is codon optimized for an onion cell. In another aspect, a nucleic acid molecule provided herein encodes a MAD7® nuclease that is codon optimized for a plant cell. In another aspect, a nucleic acid molecule provided herein encodes a MAD7® nuclease that is codon optimized for a monocotyledonous plant species. In another aspect, a nucleic acid molecule provided herein encodes a MAD7® nuclease that is codon optimized for a dicotyledonous plant species. In a further aspect, a nucleic acid molecule provided herein encodes a MAD7® nuclease that is codon optimized for a gymnosperm plant species. In a further aspect, a nucleic acid molecule provided herein encodes a MAD7® nuclease that is codon optimized for an angiosperm plant species. In a further aspect, a nucleic acid molecule provided herein encodes a MAD7® nuclease that is codon optimized for a corn cell. In a further aspect, a nucleic acid molecule provided herein encodes a MAD7® nuclease that is codon optimized for a soybean cell. In a further aspect, a nucleic acid molecule provided herein encodes a MAD7® nuclease that is codon optimized for a rice cell. In a further aspect, a nucleic acid molecule provided herein encodes a MAD7® nuclease that is codon optimized for a wheat cell. In a further aspect, a nucleic acid molecule provided herein encodes a MAD7® nuclease that is codon optimized for a cotton cell. In a further aspect, a nucleic acid molecule provided herein encodes a MAD7® nuclease that is codon optimized for a sorghum cell. In a further aspect, a nucleic acid molecule provided herein encodes a MAD7® nuclease that is codon optimized for an alfalfa cell. In a further aspect, a nucleic acid molecule provided herein encodes a MAD7® nuclease that is codon optimized for a sugar cane cell. In a further aspect, a nucleic acid molecule provided herein encodes a MAD7® nuclease that is codon optimized for an Arabidopsis cell. In a further aspect, a nucleic acid molecule provided herein encodes a MAD7® nuclease that is codon optimized for a tomato cell. In a further aspect, a nucleic acid molecule provided herein encodes a MAD7® nuclease that is codon optimized for a cucumber cell. In a further aspect, a nucleic acid molecule provided herein encodes a MAD7® nuclease that is codon optimized for a potato cell. In a further aspect, a nucleic acid molecule provided herein encodes a MAD7® nuclease that is codon optimized for an onion cell

### Guide Nucleic Acids

As used herein, a “guide nucleic acid” refers to a nucleic acid that forms a ribonucleoprotein (e.g., a complex) with a guided nuclease (e.g., without being limiting, Cas12a, CasX, MAD7®) and then guides the ribonucleoprotein to a specific sequence in a target nucleic acid molecule, where the guide nucleic acid and the target nucleic acid molecule share complementary sequences. In an aspect, a ribonucleoprotein provided herein comprises at least one guide nucleic acid.

In an aspect, a guide nucleic acid comprises DNA. In another aspect, a guide nucleic acid comprises RNA. In an aspect, a guide nucleic acid comprises DNA, RNA, or a combination thereof. In an aspect, a guide nucleic acid is single-stranded. In another aspect, a guide nucleic acid is at least partially double-stranded.

When a guide nucleic acid comprises RNA, it can be referred to as a “guide RNA.” In another aspect, a guide nucleic acid comprises DNA and RNA. In another aspect, a guide RNA is single-stranded. In another aspect, a guide RNA is double-stranded. In a further aspect, a guide RNA is partially double-stranded.

In an aspect, a guide nucleic acid comprises a guide RNA. In another aspect, a guide nucleic acid comprises at least one guide RNA. In another aspect, a guide nucleic acid comprises at least two guide RNAs. In another aspect, a guide nucleic acid comprises at least three guide RNAs. In another aspect, a guide nucleic acid comprises at least five guide RNAs. In another aspect, a guide nucleic acid comprises at least ten guide RNAs.

In another aspect, a guide nucleic acid comprises at least 10 nucleotides. In another aspect, a guide nucleic acid comprises at least 11 nucleotides. In another aspect, a guide nucleic acid comprises at least 12 nucleotides. In another aspect, a guide nucleic acid comprises at least 13 nucleotides. In another aspect, a guide nucleic acid comprises at least 14 nucleotides. In another aspect, a guide nucleic acid comprises at least 15 nucleotides. In another aspect, a guide nucleic acid comprises at least 16 nucleotides. In another aspect, a guide nucleic acid comprises at least 17 nucleotides. In another aspect, a guide nucleic acid comprises at least 18 nucleotides. In another aspect, a guide nucleic acid comprises at least 19 nucleotides. In another aspect, a guide nucleic acid comprises at least 20 nucleotides. In another aspect, a guide nucleic acid comprises at least 21 nucleotides. In another aspect, a guide nucleic acid comprises at least 22 nucleotides. In another aspect, a guide nucleic acid comprises at least 23 nucleotides. In another aspect, a guide nucleic acid comprises at least 24 nucleotides. In another aspect, a guide nucleic acid comprises at least 25 nucleotides. In another aspect, a guide nucleic acid comprises at least 26 nucleotides. In another aspect, a guide nucleic acid comprises at least 27 nucleotides. In another aspect, a guide nucleic acid comprises at least 28 nucleotides. In another aspect, a guide nucleic acid comprises at least 30 nucleotides. In another aspect, a guide nucleic acid comprises at least 35 nucleotides. In another aspect, a guide nucleic acid comprises at least 40 nucleotides. In another aspect, a guide nucleic acid comprises at least 45 nucleotides. In another aspect, a guide nucleic acid comprises at least 50 nucleotides.

In another aspect, a guide nucleic acid comprises between 10 nucleotides and 50 nucleotides. In another aspect, a guide nucleic acid comprises between 10 nucleotides and 40 nucleotides. In another aspect, a guide nucleic acid comprises between 10 nucleotides and 30 nucleotides. In another aspect, a guide nucleic acid comprises between 10 nucleotides and 20 nucleotides. In another aspect, a guide nucleic acid comprises between 16 nucleotides and 28 nucleotides. In another aspect, a guide nucleic acid comprises between 16 nucleotides and 25 nucleotides. In another aspect, a guide nucleic acid comprises between 16 nucleotides and 20 nucleotides.

In an aspect, a guide nucleic acid comprises at least 70% sequence complementarity to a target site. In an aspect, a guide nucleic acid comprises at least 75% sequence complementarity to a target site. In an aspect, a guide nucleic acid comprises at least 80% sequence complementarity to a target site. In an aspect, a guide nucleic acid comprises at least 85% sequence complementarity to a target site. In an aspect, a guide nucleic acid comprises at least 90% sequence complementarity to a target site. In an aspect, a guide nucleic acid comprises at least 91% sequence complementarity to a target site. In an aspect, a guide nucleic acid comprises at least 92% sequence complementarity to a target site. In an aspect, a guide nucleic acid comprises at least 93% sequence complementarity to a target site. In an aspect, a guide nucleic acid comprises at least 94% sequence complementarity to a target site. In an aspect, a guide nucleic acid comprises at least 95% sequence complementarity to a target site. In an aspect, a guide nucleic acid comprises at least 96% sequence complementarity to a target site. In an aspect, a guide nucleic acid comprises at least 97% sequence complementarity to a target site. In an aspect, a guide nucleic acid comprises at least 98% sequence complementarity to a target site. In an aspect, a guide nucleic acid comprises at least 99% sequence complementarity to a target site. In an aspect, a guide nucleic acid comprises 100% sequence complementarity to a target site. In another aspect, a guide nucleic acid comprises between 70% and 100% sequence complementarity to a target site. In another aspect, a guide nucleic acid comprises between 80% and 100% sequence complementarity to a target site. In another aspect, a guide nucleic acid comprises between 90% and 100% sequence complementarity to a target site.

In an aspect, a guide nucleic acid is capable of hybridizing to a target site.

As noted above, some guided nucleases, such as CasX and Cas9, require another non-coding RNA component, referred to as a trans-activating crRNA (tracrRNA), to have functional activity. Guide nucleic acid molecules provided herein can combine a crRNA and a tracrRNA into one nucleic acid molecule in what is herein referred to as a “single guide RNA” (sgRNA). The gRNA guides the active CasX complex to a target site within a target sequence, where CasX can cleave the target site. In other embodiments, the crRNA and tracrRNA are provided as separate nucleic acid molecules.

In an aspect, a guide nucleic acid comprises a crRNA. In another aspect, a guide nucleic acid comprises a tracrRNA. In a further aspect, a guide nucleic acid comprises a sgRNA.

### Target Sites

As used herein, a “target sequence” refers to a selected sequence or region of a DNA molecule in which a modification (e.g., cleavage, site-directed integration) is desired. A target sequence comprises a target site.

As used herein, a “target site” refers to the portion of a target sequence that is cleaved by a CRISPR nuclease. In contrast to a non-target nucleic acid (e.g., non-target ssDNA) or non-target region, a target site comprises significant complementarity to a guide nucleic acid or a guide RNA.

In an aspect, a target site is 100% complementary to a guide nucleic acid. In another aspect, a target site is 99% complementary to a guide nucleic acid. In another aspect, a target site is 98% complementary to a guide nucleic acid. In another aspect, a target site is 97% complementary to a guide nucleic acid. In another aspect, a target site is 96% complementary to a guide nucleic acid. In another aspect, a target site is 95% complementary to a guide nucleic acid. In another aspect, a target site is 94% complementary to a guide nucleic acid. In another aspect, a target site is 93% complementary to a guide nucleic acid. In another aspect, a target site is 92% complementary to a guide nucleic acid. In another aspect, a target site is 91% complementary to a guide nucleic acid. In another aspect, a target site is 90% complementary to a guide nucleic acid. In another aspect, a target site is 85% complementary to a guide nucleic acid. In another aspect, a target site is 80% complementary to a guide nucleic acid.

In an aspect, a target site comprises at least one PAM site. In an aspect, a target site is adjacent to a nucleic acid sequence that comprises at least one PAM site. In another aspect, a target site is within 5 nucleotides of at least one PAM site. In a further aspect, a target site is within 10 nucleotides of at least one PAM site. In another aspect, a target site is within 15 nucleotides of at least one PAM site. In another aspect, a target site is within 20 nucleotides of at least one PAM site. In another aspect, a target site is within 25 nucleotides of at least one PAM site. In another aspect, a target site is within 30 nucleotides of at least one PAM site.

In an aspect, a target site is positioned within genic DNA. In another aspect, a target site is positioned within a gene. In another aspect, a target site is positioned within a gene of interest. In another aspect, a target site is positioned within an exon of a gene. In another aspect, a target site is positioned within an intron of a gene. In another aspect, a target site is positioned within the promoter of a gene. In another aspect, a target site is positioned within 5′-UTR of a gene. In another aspect, a target site is positioned within a 3′-UTR of a gene. In another aspect, a target site is positioned within intergenic DNA

In an aspect, a target DNA molecule is single-stranded. In another aspect, a target DNA molecule is double-stranded.

In an aspect, a target sequence comprises genomic DNA. In an aspect, a target sequence is positioned within a nuclear genome. In an aspect, a target sequence comprises chromosomal DNA. In an aspect, a target sequence comprises plasmid DNA. In an aspect, a target sequence is positioned within a plasmid. In an aspect, a target sequence comprises mitochondrial DNA. In an aspect, a target sequence is positioned within a mitochondrial genome. In an aspect, a target sequence comprises plastid DNA. In an aspect, a target sequence is positioned within a plastid genome. In an aspect, a target sequence comprises chloroplast DNA. In an aspect, a target sequence is positioned within a chloroplast genome. In an aspect, a target sequence is positioned within a genome selected from the group consisting of a nuclear genome, a mitochondrial genome, and a plastid genome.

In an aspect, a target sequence comprises genic DNA. As used herein, “genic DNA” refers to DNA that encodes one or more genes. In another aspect, a target sequence comprises intergenic DNA. In contrast to genic DNA, “intergenic DNA” comprises noncoding DNA, and lacks DNA encoding a gene. In an aspect, intergenic DNA is positioned between two genes.

In an aspect, a target sequence encodes a gene. As used herein, a “gene” refers to a polynucleotide that can produce a functional unit (e.g., without being limiting, for example, a protein, or a non-coding RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a non-protein-coding RNA molecule or a precursor thereof. In another aspect, a gene encodes a protein. In some embodiments, the target sequence is selected from the group consisting of: a promoter, an enhancer sequence, a leader sequence, a transcriptional start site, a transcriptional stop site, a polyadenylation site, an exon, an intron, a splice site, a 5′-UTR, a 3′-UTR, a protein coding sequence, a non-protein-coding sequence, a miRNA, a pre-miRNA and a miRNA binding site.

Non-limiting examples of a non-protein-coding RNA molecule include a microRNA (miRNA), a miRNA precursor (pre-miRNA), a small interfering RNA (siRNA), a small RNA (18 to 26 nucleotides in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single guide RNA (sgRNA). In an aspect, a non-protein-coding RNA molecule comprises a miRNA. In an aspect, a non-protein-coding RNA molecule comprises a siRNA. In an aspect, a non-protein-coding RNA molecule comprises a ta-siRNA. In an aspect, a non-protein-coding RNA molecule is selected from the group consisting of a miRNA, a siRNA, and a ta-siRNA.

As used herein, a “gene of interest” refers to a polynucleotide sequence encoding a protein or a non-protein-coding RNA molecule that is to be integrated into a target sequence, or, alternatively, an endogenous polynucleotide sequence encoding a protein or a non-protein-coding RNA molecule that is to be edited by a ribonucleoprotein. In an aspect, a gene of interest encodes a protein. In another aspect, a gene of interest encodes a non-protein-coding RNA molecule. In an aspect, a gene of interest is exogenous to a targeted DNA molecule. In an aspect, a gene of interest replaces an endogenous gene in a targeted DNA molecule.

### Mutations

In an aspect, a ribonucleoprotein or method provided herein generates at least one mutation in a target sequence.

In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a floral cell-preferred promoter or a second nucleic acid encoding at least one guide nucleic acid operably linked to a heterologous second promoter. In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a floral tissue-preferred promoter or a second nucleic acid encoding at least one guide nucleic acid operably linked to a heterologous second promoter.

In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a heterologous promoter or a second nucleic acid encoding at least one guide nucleic acid operably linked to a floral cell-preferred promoter. In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a heterologous promoter or a second nucleic acid encoding at least one guide nucleic acid operably linked to a floral tissue-preferred promoter.

As used herein, a “mutation” refers to a non-naturally occurring alteration to a nucleic acid or amino acid sequence as compared to a naturally occurring reference nucleic acid or amino acid sequence from the same organism. It will be appreciated that, when identifying a mutation, the reference sequence should be from the same nucleic acid (e.g, gene, non-coding RNA) or amino acid (e.g, protein). In determining if a difference between two sequences comprises a mutation, it will be appreciated in the art that the comparison should not be made between homologous sequences of two different species or between homologous sequences of two different varieties of a single species. Rather, the comparison should be made between the edited (e.g., mutated) sequence and the endogenous, non-edited (e.g., “wildtype”) sequence of the same organism.

Several types of mutations are known in the art. In an aspect, a mutation comprises an insertion. An “insertion” refers to the addition of one or more nucleotides or amino acids to a given polynucleotide or amino acid sequence, respectively, as compared to an endogenous reference polynucleotide or amino acid sequence. In another aspect, a mutation comprises a deletion. A “deletion” refers to the removal of one or more nucleotides or amino acids to a given polynucleotide or amino acid sequence, respectively, as compared to an endogenous reference polynucleotide or amino acid sequence. In another aspect, a mutation comprises a substitution. A “substitution” refers to the replacement of one or more nucleotides or amino acids to a given polynucleotide or amino acid sequence, respectively, as compared to an endogenous reference polynucleotide or amino acid sequence. In another aspect, a mutation comprises an inversion. An “inversion” refers to when a segment of a polynucleotide or amino acid sequence is reversed end-to-end. In an aspect, a mutation provided herein comprises a mutation selected from the group consisting of an insertion, a deletion, a substitution, and an inversion.

In an aspect, a plant or seed comprises at least one mutation in a gene of interest, where the at least one mutation results in the deletion of one or more amino acids from a protein encoded by the gene of interest as compared to a wildtype protein.

In an aspect, a plant or seed comprises at least one mutation in a gene of interest, where the at least one mutation results in the substitution of one or more amino acids within a protein encoded by the gene of interest as compared to a wildtype protein.

In an aspect, a plant or seed comprises at least one mutation in a gene of interest, where the at least one mutation results in the insertion of one or more amino acids within a protein encoded by the gene of interest as compared to a wildtype protein.

Mutations in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a seed or plant comprises at least one mutation, where the at least one mutation results in the introduction of a premature stop codon in a messenger RNA encoded by the gene of interest as compared to a wildtype messenger RNA.

In an aspect, a mutation provided herein comprises a null mutation. As used herein, a “null mutation” refers to a mutation that confers a complete loss-of-function for a protein encoded by a gene comprising the mutation, or, alternatively, a mutation that confers a complete loss-of-function for a small RNA encoded by a genomic locus. A null mutation can cause lack of mRNA transcript production, a lack of small RNA transcript production, a lack of protein function, or a combination thereof.

A mutation provided herein can be positioned in any part of an endogenous gene. In an aspect, a mutation provided herein is positioned within an exon of an endogenous gene. In another aspect, a mutation provided herein is positioned within an intron of an endogenous gene. In a further aspect, a mutation provided herein is positioned within a 5′-untranslated region of an endogenous gene. In still another aspect, a mutation provided herein is positioned within a 3′-untranslated region of an endogenous gene. In yet another aspect, a mutation provided herein is positioned within a promoter of an endogenous gene.

In an aspect, a mutation is positioned at a splice site within a gene. A mutation at a splice site can interfere with the splicing of exons during mRNA processing. If one or more nucleotides are inserted, deleted, or substituted at a splice site, splicing can be perturbed. Perturbed splicing can result in unspliced introns, missing exons, or both, from a mature mRNA sequence. Typically, although not always, a “GU” sequence is required at the 5′ end of an intron and a “AG” sequence is required at the 3′ end of an intron for proper splicing. If either of these splice sites are mutated, splicing perturbations can occur.

In an aspect, a seed or plant comprises at least one mutation, where the at least one mutation comprises the deletion of one or more splice sites from a gene of interest. In another aspect, a seed or plant comprises at least one mutation, where the at least one mutation is positioned within one or more splice sites from a gene of interest.

In an aspect, a mutation comprises a site-directed integration. In an aspect, a site-directed integration comprises the insertion of all or part of a desired sequence into a target sequence.

As used herein, “site-directed integration” refers to all, or a portion, of a desired sequence (e.g., an exogenous gene, an edited endogenous gene) being inserted or integrated at a desired site or locus within the plant genome (e.g., target sequence). As used herein, a “desired sequence” refers to a DNA molecule comprising a nucleic acid sequence that is to be integrated into a genome of a plant or plant cell. The desired sequence can comprise a transgene or construct. In an aspect, a nucleic acid molecule comprising a desired sequence comprises one or two homology arms flanking the desired sequence to promote the targeted insertion event through homologous recombination and/or homology-directed repair.

In an aspect, a method provided herein comprises site-directed integration of a desired sequence into a target sequence.

Any site or locus within the genome of a plant can be chosen for site-directed integration of a transgene or construct of the present disclosure. In an aspect, a target sequence is positioned within a B, or supernumerary, chromosome.

For site-directed integration, a double-strand break (DSB) or nick may first be made at a target sequence via a guided nuclease or ribonucleoprotein provided herein. In the presence of a desired sequence, the DSB or nick can then be repaired by homologous recombination (HR) between the homology arm(s) of the desired sequence and the target sequence, or by non-homologous end joining (NHEJ), resulting insite-directed integration of all or part of the desired sequence into the target sequence to create the targeted insertion event at the site of the DSB or nick.

In an aspect, site-directed integration comprises the use of NHEJ repair mechanisms endogenous to a cell. In another aspect, site-directed integration comprises the use of HR repair mechanisms endogenous to a cell.

In an aspect, repair of a double-stranded break generates at least one mutation in a gene of interest as compared to a control plant of the same line or variety.

In an aspect, a mutation comprises the integration of at least 5 contiguous nucleotides of a desired sequence into a target sequence. In an aspect, a mutation comprises the integration of at least 10 contiguous nucleotides of a desired sequence molecule into a target sequence. In an aspect, a mutation comprises the integration of at least 15 contiguous nucleotides of a desired sequence into a target sequence. In an aspect, a mutation comprises the integration of at least 20 contiguous nucleotides of a desired sequence into a target sequence. In an aspect, a mutation comprises the integration of at least 25 contiguous nucleotides of a desired sequence into a target sequence. In an aspect, a mutation comprises the integration of at least 50 contiguous nucleotides of a desired sequence into a target sequence. In an aspect, a mutation comprises the integration of at least 100 contiguous nucleotides of a desired sequence into a target sequence. In an aspect, a mutation comprises the integration of at least 250 contiguous nucleotides of a desired sequence into a target sequence. In an aspect, a mutation comprises the integration of at least 500 contiguous nucleotides of a desired sequence into a target sequence. In an aspect, a mutation comprises the integration of at least 1000 contiguous nucleotides of a desired sequence into a target sequence. In an aspect, a mutation comprises the integration of at least 2000 contiguous nucleotides of a desired sequence into a target sequence.

In an aspect, a mutation comprises the integration of between 5 contiguous nucleotides and 3500 contiguous nucleotides of a desired sequence into a target sequence. In an aspect, a mutation comprises the integration of between 5 contiguous nucleotides and 2500 contiguous nucleotides of a desired sequence into a target sequence. In an aspect, a mutation comprises the integration of between 5 contiguous nucleotides and 1500 contiguous nucleotides of a desired sequence into a target sequence. In an aspect, a mutation comprises the integration of between 5 contiguous nucleotides and 750 contiguous nucleotides of a desired sequence into a target sequence. In an aspect, a mutation comprises the integration of between 5 contiguous nucleotides and 500 contiguous nucleotides of a desired sequence into a target sequence. In an aspect, a mutation comprises the integration of between 5 contiguous nucleotides and 250 contiguous nucleotides of a desired sequence into a target sequence. In an aspect, a mutation comprises the integration of between 5 contiguous nucleotides and 150 contiguous nucleotides of a desired sequence into a target sequence. In an aspect, a mutation comprises the integration of between 25 contiguous nucleotides and 2500 contiguous nucleotides of a desired sequence into a target sequence. In an aspect, a mutation comprises the integration of between 25 contiguous nucleotides and 1500 contiguous nucleotides of a desired sequence into a target sequence. In an aspect, a mutation comprises the integration of between 25 contiguous nucleotides and 750 contiguous nucleotides of a desired sequence into a target sequence. In an aspect, a mutation comprises the integration of between 50 contiguous nucleotides and 2500 contiguous nucleotides of a desired sequence into a target sequence. In an aspect, a mutation comprises the integration of between 50 contiguous nucleotides and 1500 contiguous nucleotides of a desired sequence into a target sequence. In an aspect, a mutation comprises the integration of between 50 contiguous nucleotides and 750 contiguous nucleotides of a desired sequence into a target sequence. In an aspect, a mutation comprises the integration of between 100 contiguous nucleotides and 2500 contiguous nucleotides of a desired sequence into a target Sequence. In an aspect, a mutation comprises the integration of between 100 contiguous nucleotides and 1500 contiguous nucleotides of a desired sequence into a target Sequence. In an aspect, a mutation comprises the integration of between 100 contiguous nucleotides and 750 contiguous nucleotides of a desired sequence into a target Sequence.

In an aspect, a method provided herein further comprises detecting an edit or a mutation in a target sequence. The screening and selection of mutagenized or edited plants or plant cells can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the above-referenced techniques are known in the art.

In an aspect, a sequence provided herein encodes at least one ribozyme. In an aspect, a sequence provided herein encodes at least two ribozymes. In an aspect, a ribozyme is a self-cleaving ribozyme. Self-cleaving ribozymes are known in the art. For example, see Jimenez et al., Trends Biochem. Sci., 40:648-661 (2015).

In an aspect, a sequence encoding at least one guide nucleic acid is flanked by self-cleaving ribozymes. In an aspect, a sequence encoding at least one guide nucleic acid is immediately adjacent to a sequence encoding a ribozyme (e.g., the 5′-most nucleotide of the guide nucleic acid abuts the 3′-most nucleotide of the ribozyme or the 3′-most nucleotide of the guide nucleic acid abuts the 5′-most nucleotide of the ribozyme). In an aspect, a sequence encoding at least one guide nucleic acid is separated from a sequence encoding a ribozyme by at least 1, at least 2, at least 3, at least 4, at least 5, at least 10, at least 20, at least 30, at least 40, at least 50, at least 100, at least 250, at least 500, or at least 10000 nucleotides.

### Plants

Any plant or plant cell can be used with the methods and compositions provided herein.

In an aspect, a plant is selected from the group consisting of a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, and a potato plant. In an aspect, a plant is an angiosperm. In an aspect, a plant is a gymnosperm. In an aspect, a plant is a monocotyledonous plant. In an aspect, a plant is a dicotyledonous plant. In an aspect, a plant is a plant of a family selected from the group consisting of Alliaceae, Anacardiaceae, Apiaceae, Arecaceae, Asteraceae, Brassicaceae, Caesalpiniaceae, Cucurbitaceae, Ericaceae, Fabaceae, Juglandaceae, Malvaceae, Mimosaceae, Moraceae, Musaceae, Orchidaceae, Papilionaceae, Pinaceae, Poaceae, Rosaceae, Rutaceae, Rubiaceae, and Solanaceae.

In an aspect, a plant cell is selected from the group consisting of a corn cell, a rice cell, a sorghum cell, a wheat cell, an alfalfa cell, a barley cell, a millet cell, a rye cell, a sugarcane cell, a cotton cell, a soybean cell, a canola cell, a tomato cell, an onion cell, a cucumber cell, an Arabidopsis cell, and a potato cell. In an aspect, a plant cell is an angiosperm plant cell. In an aspect, a plant cell is a gymnosperm plant cell. In an aspect, a plant cell is a monocotyledonous plant cell. In an aspect, a plant cell is a dicotyledonous plant cell. In an aspect, a plant cell is a plant cell of a family selected from the group consisting of Alliaceae, Anacardiaceae, Apiaceae, Arecaceae, Asteraceae, Brassicaceae, Caesalpiniaceae, Cucurbitaceae, Ericaceae, Fabaceae, Juglandaceae, Malvaceae, Mimosaceae, Moraceae, Musaceae, Orchidaceae, Papilionaceae, Pinaceae, Poaceae, Rosaceae, Rutaceae, Rubiaceae, and Solanaceae.

As used herein, a “variety” refers to a group of plants within a species (e.g., without being limiting Zea mays) that share certain genetic traits that separate them from other possible varieties within that species. Varieties can be inbreds or hybrids, though commercial plants are often hybrids to take advantage of hybrid vigor. Individuals within a hybrid cultivar are homogeneous, nearly genetically identical, with most loci in the heterozygous state.

As used herein, the term “inbred” means a line that has been bred for genetic homogeneity. In an aspect, a seed provided herein is an inbred seed. In an aspect, a plant provided herein is an inbred plant.

As used herein, the term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines. In an aspect, a seed provided herein is a hybrid seed. In an aspect, a plant provided herein is a hybrid plant.

### Transformation

Methods can involve transient transformation or stable integration of any nucleic acid molecule into any plant or plant cell provided herein.

As used herein, “stable integration” or “stably integrated” refers to a transfer of DNA into genomic DNA of a targeted cell or plant that allows the targeted cell or plant to pass the transferred DNA to the next generation of the transformed organism. Stable transformation requires the integration of transferred DNA within the reproductive cell(s) of the transformed organism. As used herein, “transiently transformed” or “transient transformation” refers to a transfer of DNA into a cell that is not transferred to the next generation of the transformed organism. In a transient transformation the transformed DNA does not typically integrate into the transformed cell's genomic DNA. In one aspect, a method stably transforms a plant cell or plant with one or more nucleic acid molecules provided herein. In another aspect, a method transiently transforms a plant cell or plant with one or more nucleic acid molecules provided herein.

In an aspect, a nucleic acid molecule encoding a guided nuclease is stably integrated into a genome of a plant. In an aspect, a nucleic acid molecule encoding a Cas12a nuclease is stably integrated into a genome of a plant. In an aspect, a nucleic acid molecule encoding a CasX nuclease is stably integrated into a genome of a plant. In an aspect, a nucleic acid molecule encoding a MAD7® nuclease is stably integrated into a genome of a plant. In an aspect, a nucleic acid molecule encoding a guide nucleic acid is stably integrated into a genome of a plant. In an aspect, a nucleic acid molecule encoding a guide RNA is stably integrated into a genome of a plant. In an aspect, a nucleic acid molecule encoding a single-guide RNA is stably integrated into a genome of a plant.

Numerous methods for transforming cells with a recombinant nucleic acid molecule or construct are known in the art, which can be used according to methods of the present application. Any suitable method or technique for transformation of a cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants.

In an aspect, a method comprises providing a cell with a nucleic acid molecule via Agrobacterium-mediated transformation. In an aspect, a method comprises providing a cell with a nucleic acid molecule via polyethylene glycol-mediated transformation. In an aspect, a method comprises providing a cell with a nucleic acid molecule via biolistic transformation. In an aspect, a method comprises providing a cell with a nucleic acid molecule via liposome-mediated transfection. In an aspect, a method comprises providing a cell with a nucleic acid molecule via viral transduction. In an aspect, a method comprises providing a cell with a nucleic acid molecule via use of one or more delivery particles. In an aspect, a method comprises providing a cell with a nucleic acid molecule via microinjection. In an aspect, a method comprises providing a cell with a nucleic acid molecule via electroporation.

In an aspect, a nucleic acid molecule is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

Delivery vehicles, vectors, particles, nanoparticles, formulations and components thereof for expression of one or more elements of a nucleic acid molecule are as used in WO 2014/093622. In an aspect, a method of providing a nucleic acid molecule or a protein to a cell comprises delivery via a delivery particle. In an aspect, a method of providing a nucleic acid molecule to a plant cell or plant comprises delivery via a delivery vesicle. In an aspect, a delivery vesicle is selected from the group consisting of an exosome and a liposome. In an aspect, a method of providing a nucleic acid molecule to a plant cell or plant comprises delivery via a viral vector. In an aspect, a viral vector is selected from the group consisting of an adenovirus vector, a lentivirus vector, and an adeno-associated viral vector. In another aspect, a method providing a nucleic acid molecule to a plant cell or plant comprises delivery via a nanoparticle. In an aspect, a method providing a nucleic acid molecule to a plant cell or plant comprises microinjection. In an aspect, a method providing a nucleic acid molecule to a plant cell or plant comprises polycations. In an aspect, a method providing a nucleic acid molecule to a plant cell or plant comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of nucleic acids into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning includes (a) surfactants, (b) organic solvents, aqueous solutions, or aqueous mixtures of organic solvents, (c) oxidizing agents, (e) acids, (f) bases, (g) oils, (h) enzymes, or combinations thereof.

Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e. g., plant-sourced oils, crop oils (such as those listed in the 9^{th} Compendium of Herbicide Adjuvants, publicly available on line at www(dot)herbicide(dot)adjuvants(dot)com) can be used, e. g., paraffinic oils, polyol fatty acid esters, or oils with short-chain molecules modified with amides or polyamines such as polyethyleneimine or N-pyrrolidine.

Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e. g., trisiloxane ethoxylate surfactants or a silicone polyether copolymer such as a copolymer of polyalkylene oxide modified heptamethyl trisiloxane and allyloxypolypropylene glycol methylether (commercially available as Silwet® L-77).

Useful physical agents can include (a) abrasives such as carborundum, corundum, sand, calcite, pumice, garnet, and the like, (b) nanoparticles such as carbon nanotubes or (c) a physical force. Carbon nanotubes are disclosed by Kam et al. (2004) J. Am. Chem. Soc, 126 (22):6850-6851, Liu et al. (2009) Nano Lett, 9(3): 1007-1010, and Khodakovskaya et al. (2009) ACS Nano, 3(10):3221-3227. Physical force agents can include heating, chilling, the application of positive pressure, or ultrasound treatment. Embodiments of the method can optionally include an incubation step, a neutralization step (e.g., to neutralize an acid, base, or oxidizing agent, or to inactivate an enzyme), a rinsing step, or combinations thereof. The methods of the invention can further include the application of other agents which will have enhanced effect due to the silencing of certain genes. For example, when a polynucleotide is designed to regulate genes that provide herbicide resistance, the subsequent application of the herbicide can have a dramatic effect on herbicide efficacy.

Agents for laboratory conditioning of a plant cell to permeation by polynucleotides include, e.g., application of a chemical agent, enzymatic treatment, heating or chilling, treatment with positive or negative pressure, or ultrasound treatment. Agents for conditioning plants in a field include chemical agents such as surfactants and salts.

In an aspect, a transformed or transfected cell is a plant cell. Recipient plant cell or explant targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a guard cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

The following non-limiting embodiments are specifically envisioned:

1. A plant comprising:
(a) a first nucleic acid sequence encoding a guided nuclease capable of generating a staggered cut in a double-stranded DNA molecule operably linked to a heterologous floral tissue-preferred promoter or floral cell-preferred promoter; and
(b) a second nucleic acid sequence encoding at least one guide nucleic acid operably linked to a heterologous second promoter, wherein the at least one guide nucleic acid is capable of hybridizing to a target sequence within a genome of the plant; or
(c) a first nucleic acid sequence encoding a guided nuclease capable of generating a staggered cut in a double-stranded DNA molecule operably linked to a first heterologous promoter; and
(d) a second nucleic acid sequence encoding at least one guide nucleic acid operably linked to a heterologous floral tissue-preferred promoter or floral cell-preferred promoter; or
(e) a first nucleic acid sequence encoding a guided nuclease capable of generating a staggered cut in a double-stranded DNA molecule operably linked to a heterologous floral tissue-preferred promoter or floral cell-preferred promoter; and
(f) a second nucleic acid sequence encoding at least one guide nucleic acid operably linked to a heterologous floral tissue-preferred promoter or floral cell-preferred promoter.

2. The plant of embodiment 1, wherein the guided nuclease is selected from the group consisting of Cas12a, MAD7® and CasX.

3. The plant of embodiment 2, wherein the Cas12a is selected from the group consisting of LbCas12a and FnCas12a.

4. The plant of embodiment 2, wherein the first nucleic acid sequence comprises a nucleic acid sequence at least 90% identical to SEQ ID NO: 32 or SEQ ID NO: 36.

5. The plant of any one of embodiments 1-4, wherein the first nucleic acid sequence is codon-optimized for the plant.

6. The plant of any one of embodiments 1-5, wherein the first nucleic acid sequence encodes at least one nuclear localization signal.

7. The plant of embodiment 6, wherein the at least one nuclear localization signal comprises a nucleic acid sequence selected from the group consisting of SEQ ID NOs: 33 and 34.

8. The plant of any one of embodiments 1-7, wherein the floral cell-preferred promoter is a floral cell-specific promoter.

9. The plant of any one of embodiments 1-8, wherein the floral tissue-preferred promoter is a floral tissue-specific promoter.

10. The plant of any one of embodiments 1-9, wherein the floral cell-preferred promoter is selected from the group consisting of an A gene promoter, a B gene promoter, a C gene promoter, a D gene promoter, and an E gene promoter.

11. The plant of any one of embodiments 1-10, wherein the floral cell-preferred promoter or floral tissue-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

12. The plant of any one of embodiments 1-11, wherein the floral cell-preferred promoter or floral tissue-preferred promoter comprises a nucleic acid sequence at least 90% identical to a nucleic acid sequence selected from the group consisting of SEQ ID NOs: 1-30 or selected from the group consisting of SEQ ID NOs: 1-16, 18-19, 21-30, 45-49, or a functional fragment thereof.

13. The plant of any one of embodiments 1-12, wherein the first or second promoter is selected from the group consisting of a tissue-preferred promoter, a tissue-specific promoter, an inducible promoter, and a constitutive promoter.

14. The plant of any one of embodiments 1-13, wherein the first or second promoter is a floral cell-preferred promoter or a floral tissue-preferred promoter.

15. The plant of any one of embodiments 1-14, wherein the first or second promoter is floral cell-specific promoter or a floral tissue-specific promoter.

16. The plant of any one of embodiments 1-15, wherein the first or second promoter is selected from the group consisting of an A gene promoter, a B gene promoter, a C gene promoter, a D gene promoter, and an E gene promoter.

17. The plant of any one of embodiments 1-16, wherein the first or second promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

18. The plant of any one of embodiments 1-17, wherein the first or second promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

19. The plant of embodiment 13, wherein the constitutive promoter is selected from the group consisting of a DaMV promoter, a CaMV 35S promoter, an Actin promoter, a Rab15 promoter, and a Ubiquitin promoter.

20. The plant of any one of embodiments 1-19, wherein the at least one guide nucleic acid comprises at least one guide RNA.

21. The plant of any one of embodiments 1-20, wherein the first nucleic acid sequence, the second nucleic acid sequence, or both, are stably integrated into a genome of the plant.

22. The plant of any one of embodiments 1-21, wherein the guided nuclease and the at least one guide RNA form a ribonucleoprotein in a floral cell.

23. The plant of embodiment 22, wherein the ribonucleoprotein generates at least one double-stranded break within the target site in a floral cell.

24. The plant of any one of embodiments 1-23, wherein the plant is selected from the group consisting of a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, and a potato plant.

25. The plant of any one of embodiments 1-24, wherein the genome is selected from the group consisting of a nuclear genome, a mitochondrial genome, and a plastid genome.

26. A seed produced by the plant of any one of embodiments 1-25, optionally comprising a modification at or near the target sequence in the plant genome.

27. The seed of embodiment 26, wherein the seed comprises at least one mutation in a gene of interest comprising the target sequence as compared to a seed from a control plant of the same variety that lacks the first nucleic acid sequence or second nucleic acid sequence.

28. The seed of embodiment 26, wherein the at least one mutation in the gene of interest results in the deletion of one or more amino acids from a protein encoded by the gene of interest as compared to a wild-type protein.

29. The seed of embodiment 26, wherein the at least one mutation in the gene of interest results in the substitution of one or more amino acids within a protein encoded by the gene of interest as compared to a wild-type protein.

30. The seed of embodiment 26, wherein the at least one mutation in the gene of interest results in the introduction of a premature stop codon in a messenger RNA encoded by the gene of interest as compared to a wildtype messenger RNA.

31. The seed of embodiment 26, wherein the at least one mutation in the gene of interest comprises the deletion of one or more splice sites from the gene of interest.

32. The seed of any one of embodiments 26-31, wherein the seed is a hybrid seed.

33. The seed of any one of embodiments 26-31, wherein the seed is an inbred seed.

34. A method of editing a genome of a plant comprising:
(a) introducing into a plant cell:
   (i) a first nucleic acid sequence encoding a guided nuclease capable of generating a staggered cut in a double-stranded DNA molecule operably linked to a heterologous floral cell-preferred promoter; and
   (ii) a second nucleic acid sequence encoding at least one guide nucleic acid operably linked to a heterologous second promoter, wherein the at least one guide nucleic acid is capable of hybridizing to a target sequence within the genome; and
(b) regenerating at least one plant from the plant cell of step (a), wherein the guided nuclease and at least one guide nucleic acid form a ribonucleoprotein within at least one floral cell of the plant, and wherein the ribonucleoprotein generates at least one double-stranded break within the target sequence in the at least one floral cell.

35. A method of editing a genome of a plant cell comprising:
(a) crossing a first plant with a second plant, wherein the first plant comprises a first nucleic acid sequence encoding a guided nuclease capable of generating a staggered cut in a double-stranded DNA molecule operably linked to a heterologous floral tissue-preferred promoter, and wherein the second plant comprises a second nucleic acid sequence encoding at least one guide nucleic acid operably linked to a heterologous second promoter, wherein the at least one guide nucleic acid is capable of hybridizing to a target sequence within the genome; and
(b) obtaining at least one embryo from the crossing of step (a), wherein the guided nuclease and the at least one guide nucleic acid form a ribonucleoprotein within the at least one embryo, and wherein the ribonucleoprotein generates at least one double-stranded break within the target sequence in the at least one embryo.

36. A method of editing a genome of a plant comprising:
(a) introducing into a plant cell:
   (i) a first nucleic acid sequence encoding a guided nuclease capable of generating a staggered cut in a double-stranded DNA molecule operably linked to a heterologous floral tissue-preferred promoter; and
   (ii) a second nucleic acid sequence encoding at least one guide nucleic acid operably linked to a heterologous second promoter, wherein the at least one guide nucleic acid is capable of hybridizing to a target sequence within the genome;
(b) regenerating at least one plant from the plant cell of step (a); and
(c) fertilizing the at least one plant to create at least one embryo, wherein the guided nuclease and at least one guide nucleic acid form a ribonucleoprotein within the at least one embryo from step (c), and wherein the ribonucleoprotein generates at least one double-stranded break within the target sequence in the at least one embryo.

37. A method of generating a site-directed integration in a plant comprising:
(a) introducing into a plant cell:
   (i) a first nucleic acid sequence encoding a guided nuclease capable of generating a staggered cut in a double-stranded DNA molecule operably linked to a heterologous floral cell-preferred promoter;
   (ii) a second nucleic acid sequence encoding one or more guide nucleic acids operably linked to a heterologous second promoter, wherein the one or more guide nucleic acids are
      (A) capable of hybridizing to a target sequence within a genome of the plant; and
      (B) capable of hybridizing to a first site and a second site flanking a nucleic acid sequence encoding a gene of interest; and
   (iii) a third nucleic acid sequence encoding the gene of interest; and
(b) regenerating at least one plant from the plant cell of step (a);
wherein the guided nuclease and at least one guide RNA form a ribonucleoprotein within at least one floral cell of the plant, wherein the ribonucleoprotein generates a double-stranded break within the target sequence molecule, the first site, and the second site, and wherein the gene of interest is integrated into the target sequence in the at least one floral cell.

38. A method of generating a site-directed integration in a plant comprising:
(a) introducing into a plant cell:
   (i) a first nucleic acid sequence encoding a guided nuclease capable of generating a staggered cut in a double-stranded DNA molecule operably linked to a heterologous floral tissue-preferred promoter;
   (ii) a second nucleic acid sequence encoding one or more guide nucleic acids operably linked to a heterologous second promoter, wherein the one or more guide nucleic acids are
      (A) capable of hybridizing to a target sequence within a genome of the plant; and
      (B) capable of hybridizing to a first site and a second site flanking a nucleic acid sequence encoding a gene of interest;
   (iii) a third nucleic acid sequence encoding the gene of interest;
(b) regenerating at least one plant from the plant cell of step (a); and
(c) fertilizing the at least one plant from step (b) to create at least one embryo; wherein the guided nuclease and at least one guide RNA form a ribonucleoprotein within at least one embryo, wherein the ribonucleoprotein generates a double-stranded break within the target DNA molecule, the first site, and the second site, and wherein the gene of interest is integrated into the target sequence in the at least one embryo.

39. A method of editing a genome of a plant comprising:
(a) introducing into a plant cell:
   (i) a first nucleic acid sequence encoding a guided nuclease capable of generating a staggered cut in a double-stranded DNA molecule operably linked to a heterologous first promoter; and
   (ii) a second nucleic acid sequence encoding at least one guide nucleic acid operably linked to a heterologous floral cell-preferred or floral tissue-preferred promoter, wherein the at least one guide nucleic acid is capable of hybridizing to a target sequence within the genome; and
(b) regenerating at least one plant from the plant cell of step (a), wherein the guided nuclease and at least one guide nucleic acid form a ribonucleoprotein within at least one floral cell of the plant, and wherein the ribonucleoprotein generates at least one double-stranded break within the target sequence in the at least one floral cell.

40. A method of editing a genome of a plant cell comprising:
(a) crossing a first plant with a second plant, wherein the first plant comprises a first nucleic acid sequence encoding a guided nuclease capable of generating a staggered cut in a double-stranded DNA molecule operably linked to a heterologous floral tissue-preferred promoter, and wherein the second plant comprises a second nucleic acid sequence encoding at least one guide nucleic acid operably linked to a heterologous second promoter, wherein the at least one guide nucleic acid is capable of hybridizing to a target sequence within the genome; and
(b) obtaining at least one progeny plant from the crossing of step (a), wherein the guided nuclease and the at least one guide nucleic acid form a ribonucleoprotein within at least one floral cell, and wherein the ribonucleoprotein generates at least one double-stranded break within the target sequence in the at least one floral cell.

41. A method of editing a genome of a plant cell comprising:
(a) crossing a first plant with a second plant, wherein the first plant comprises a first nucleic acid sequence encoding a guided nuclease capable of generating a staggered cut in a double-stranded DNA molecule operably linked to a heterologous floral cell-preferred promoter, and wherein the second plant comprises a second nucleic acid sequence encoding at least one guide nucleic acid operably linked to a heterologous second promoter, wherein the at least one guide nucleic acid is capable of hybridizing to a target sequence within the genome; and
(b) obtaining at least one progeny plant from the crossing of step (a), wherein the guided nuclease and the at least one guide nucleic acid form a ribonucleoprotein within at least one floral cell, and wherein the ribonucleoprotein generates at least one double-stranded break within the target sequence in the at least one floral cell.

42. A method of generating a site-directed integration in a plant comprising:
(a) introducing into a plant cell:
   (i) a first nucleic acid sequence encoding a guided nuclease capable of generating a staggered cut in a double-stranded DNA molecule operably linked to a heterologous first promoter;
   (ii) a second nucleic acid sequence encoding one or more guide nucleic acids operably linked to a heterologous floral cell-preferred or floral tissue-preferred promoter, wherein the one or more guide nucleic acids are
      a. capable of hybridizing to a target sequence within a genome of the plant; and
      b. capable of hybridizing to a first site and a second site flanking a nucleic acid sequence encoding a gene of interest; and
   (iii) a third nucleic acid sequence encoding the gene of interest; and
(b) regenerating at least one plant from the plant cell of step (a); wherein the guided nuclease and at least one guide RNA form a ribonucleoprotein within at least one floral cell of the plant, wherein the ribonucleoprotein generates a double-stranded break within the target sequence molecule, the first site, and the second site, and wherein the gene of interest is integrated into the target sequence in the at least one floral cell.

43. The method of any one of embodiments 34-42, wherein the target sequence comprises genic DNA.

44. The method of any one of embodiments 34-42, wherein the target sequence comprises intergenic DNA.

45. The method of any one of embodiments 34-36 and 39-41, wherein the target sequence is within a gene of interest.

46. The method of embodiment 45, wherein the gene of interest encodes a protein or a non-protein-coding RNA.

47. The method of embodiment 46, wherein the non-protein-coding RNA is selected from the group consisting of a microRNA, a small interfering RNA (siRNA), a trans-acting siRNA, or a precursor thereof.

48. The method of any one of embodiments 34-42, wherein the guided nuclease is selected from the group consisting of Cas12a, MAD7® and CasX.

49. The method of embodiment 48, wherein the Cas12a is selected from the group consisting of LbCas12a and FnCas12a.

50. The method of any one of embodiments 34, 37, or 39-42, wherein the floral cell-preferred promoter is a floral cell-specific promoter.

51. The method of any one of embodiments 35, 36, or 38, wherein the floral tissue-preferred promoter is a floral tissue-specific promoter.

52. The method of any one of embodiments 34, 37, or 39-42, wherein the floral cell-preferred promoter is selected from the group consisting of an A gene promoter, a B gene promoter, a C gene promoter, a D gene promoter, and an E gene promoter.

53. The method of any one of embodiments 34, 37, or 39-42, wherein the floral cell-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

54. The method of any one of embodiments 34, 37, or 39-42, wherein the floral cell-preferred promoter comprises a nucleic acid sequence at least 90% identical to a nucleic acid sequence selected from the group consisting of SEQ ID NOs: 1-30 or selected from the group consisting of SEQ ID NOs: 1-16, 18-19, 21-30, 45-49, or a functional fragment thereof.

55. The method of any one of embodiments 35, 36, or 38, wherein the floral tissue-preferred promoter is selected from the group consisting of an A gene promoter, a B gene promoter, a C gene promoter, a D gene promoter, and an E gene promoter.

56. The method of any one of embodiments 35, 36, or 38, wherein the floral cell-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

57. The method of any one of embodiments 35, 36, or 38, wherein the floral tissue-preferred promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

58. The method of any one of embodiments 34-42, wherein the first or second promoter is selected from the group consisting of a tissue-preferred promoter, a tissue-specific promoter, an inducible promoter, and a constitutive promoter.

59. The method of any one of embodiments 34-42, wherein the first or second promoter is a floral cell-preferred promoter or a floral tissue-preferred promoter.

60. The method of embodiment 59, wherein the floral cell-preferred promoter or floral tissue-preferred promoter is selected from the group consisting of an A gene promoter, a B gene promoter, a C gene promoter, a D gene promoter, and an E gene promoter.

61. The method of embodiment 59 or 60, wherein the floral cell-preferred promoter or floral tissue-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

62. The method of any one of embodiments 59-61, wherein the floral cell-preferred promoter or floral tissue-preferred promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

63. The method of embodiment 58, wherein the constitutive promoter is selected from the group consisting of a CAMV35S promoter, an Actin promoter, a Rab15 promoter, and a Ubiquitin promoter.

64. The method of any one of embodiments 34-42, wherein the one or more guide nucleic acids comprises at least one guide RNA.

65. The method of any one of embodiments 34-42, wherein the first nucleic acid sequence, the second nucleic acid sequence, or both, are stably integrated into a genome of the plant.

66. The method of any one of embodiments 34-42, wherein the plant is selected from the group consisting of corn, rice, sorghum, wheat, alfalfa, barley, millet, rye, sugarcane, cotton, soybean, canola, tomato, and potato.

67. The method of any one of embodiments 34-42, wherein the genome is selected from the group consisting of a nuclear genome, a mitochondrial genome, and a plastid genome.

68. The method of any one of embodiments 34-36 or 39-41, wherein repair of the double-stranded break generates at least one mutation in the target sequence as compared to a control plant of the same line or variety that lacks the first nucleic acid sequence or second nucleic acid sequence, optionally wherein the mutation results in the deletion, insertion or substitution of at least one nucleotide at or near the target sequence.

69. The method of embodiment 68, wherein the at least one mutation in the target sequence results in the deletion of one or more amino acids from a protein encoded by a gene of interest as compared to a wild-type protein.

70. The method of embodiment 68, wherein the at least one mutation in the target sequence results in the substitution of one or more amino acids within a protein encoded by a gene of interest as compared to a wild-type protein.

71. The method of embodiment 68, wherein the at least one mutation in the target sequence results in the introduction of a premature stop codon in a messenger RNA encoded by a gene of interest as compared to a wild-type messenger RNA.

72. The method of embodiment 68, wherein the at least one mutation in the target sequence comprises the deletion of one or more splice sites from a gene of interest.

73. A recombinant DNA construct comprising (a) a first nucleic acid sequence encoding a guided nuclease capable of generating a staggered cut in a double-stranded DNA molecule operably linked to a heterologous floral cell-preferred or floral tissue-preferred promoter; and (b) a second nucleic acid sequence encoding at least one guide nucleic acid operably linked to a heterologous second promoter; or comprising (c) a first nucleic acid sequence encoding a guided nuclease capable of generating a staggered cut in a double-stranded DNA molecule operably linked to a heterologous promoter; and (d) a second nucleic acid sequence encoding at least one guide nucleic acid operably linked to a heterologous promoter floral cell-preferred or floral tissue-preferred promoter wherein the at least one guide nucleic acid is capable of hybridizing to a target sequence within a genome of a plant

74. The recombinant DNA construct of embodiment 73, wherein the sequence encoding the at least one guide nucleic acid is flanked by self-cleaving ribozymes.

75. A method of generating two or more progeny plants with unique edits from a single transformed plant cell, the method comprising:
(a) introducing into the plant cell:
   (i) a first nucleic acid sequence encoding a guided nuclease capable of generating a staggered cut in a double-stranded DNA molecule operably linked to a heterologous floral cell-preferred promoter; and
   (ii) a second nucleic acid sequence encoding at least one guide nucleic acid operably linked to a heterologous second promoter, wherein the at least one guide nucleic acid is capable of hybridizing to a target sequence within the genome; and
(b) regenerating a first plant from the plant cell of step (a), wherein the guided nuclease and at least one guide nucleic acid form a ribonucleoprotein within at least one floral cell of the first plant, and wherein the ribonucleoprotein generates at least one double-stranded break within the target sequence in the at least one floral cell;
(c) pollinating the first plant of step (b); and
(d) germinating two or more seeds produced from step (c) to produce two or more progeny plants with unique edits.

76. A method of generating two or more progeny plants with unique edits from a single transformed plant cell, the method comprising:
(a) introducing into the plant cell:
   (i) a first nucleic acid sequence encoding a guided nuclease capable of generating a staggered cut in a double-stranded DNA molecule operably linked to a heterologous first promoter; and
   (ii) a second nucleic acid sequence encoding at least one guide nucleic acid operably linked to a heterologous floral cell-preferred promoter, wherein the at least one guide nucleic acid is capable of hybridizing to a target sequence within the genome; and
(b) regenerating a first plant from the plant cell of step (a), wherein the guided nuclease and at least one guide nucleic acid form a ribonucleoprotein within at least one floral cell of the first plant, and wherein the ribonucleoprotein generates at least one double-stranded break within the target sequence in the at least one floral cell;
(c) pollinating the first plant of step (b); and
(d) germinating two or more seeds produced from step (c) to produced two or more progeny plants with unique edits.

77. A method of generating two or more progeny plants with unique edits from a single transformed plant cell, the method comprising:
(a) introducing into the plant cell:
   (i) a first nucleic acid sequence encoding a guided nuclease capable of generating a staggered cut in a double-stranded DNA molecule operably linked to a heterologous floral tissue-preferred promoter; and
   (ii) a second nucleic acid sequence encoding at least one guide nucleic acid operably linked to a heterologous second promoter, wherein the at least one guide nucleic acid is capable of hybridizing to a target sequence within the genome; and
(b) regenerating a first plant from the plant cell of step (a);
(c) pollinating the first plant of step (b), wherein the guided nuclease and at least one guide nucleic acid form a ribonucleoprotein within a floral tissue, and wherein the ribonucleoprotein generates at least one double-stranded break within the target sequence in the floral tissue; and
(d) germinating two or more seeds produced from step (c) to produced two or more progeny plants with unique edits.

78. A method of generating two or more progeny plants with unique edits from a single transformed plant cell, the method comprising:
(a) introducing into the plant cell:
   (i) a first nucleic acid sequence encoding a guided nuclease capable of generating a staggered cut in a double-stranded DNA molecule operably linked to a heterologous first promoter; and
   (ii) a second nucleic acid sequence encoding at least one guide nucleic acid operably linked to a heterologous floral tissue-preferred promoter, wherein the at least one guide nucleic acid is capable of hybridizing to a target sequence within the genome; and
(b) regenerating a first plant from the plant cell of step (a);
(c) pollinating the first plant of step (b), wherein the guided nuclease and at least one guide nucleic acid form a ribonucleoprotein within a floral tissue, and wherein the ribonucleoprotein generates at least one double-stranded break within the target sequence in the floral tissue; and
(d) germinating two or more seeds produced from step (c) to produced two or more progeny plants with unique edits.

79. The method of any one of embodiments 75-78, wherein the target sequence comprises genic DNA.

80. The method of any one of embodiments 75-78, wherein the target sequence comprises intergenic DNA.

81. The method of any one of embodiments 75-78, wherein the target sequence is within a gene of interest.

82. The method of embodiment 81, wherein the gene of interest encodes a protein or a non-protein-coding RNA.

83. The method of embodiment 82, wherein the non-protein-coding RNA is selected from the group consisting of a microRNA, a small interfering RNA (siRNA), a trans-acting siRNA, or a precursor thereof.

84. The method of any one of embodiments 75-78, wherein the guided nuclease is selected from the group consisting of Cas12a, MAD7® and CasX.

85. The method of embodiment 84, wherein the Cas12a is selected from the group consisting of LbCas12a and FnCas12a.

86. The method of embodiment 75 or 76, wherein the floral cell-preferred promoter is a floral cell-specific promoter.

87. The method of any one of embodiments 77 or 78, wherein the floral tissue-preferred promoter is a floral tissue-specific promoter.

88. The method of embodiments 75 or 76, wherein the floral cell-preferred promoter is selected from the group consisting of an A gene promoter, a B gene promoter, a C gene promoter, a D gene promoter, and an E gene promoter.

89. The method of embodiments 75 or 76, wherein the floral cell-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

90. The method of embodiments 75 or 76, wherein the floral cell-preferred promoter comprises a nucleic acid sequence at least 90% identical to a nucleic acid sequence selected from the group consisting of SEQ ID NOs:1-30, or selected from the group consisting of SEQ ID NOs: 1-16, 18-19, 21-30, 45-49, or a functional fragment thereof.

91. The method of embodiments 77 or 78, wherein the floral tissue-preferred promoter is selected from the group consisting of an A gene promoter, a B gene promoter, a C gene promoter, a D gene promoter, and an E gene promoter.

92. The method of embodiments 77 or 78, wherein the floral tissue-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

93. The method of embodiments 77 or 78, wherein the floral tissue-preferred promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

94. The method of embodiments 75 or 77, wherein the second promoter is selected from the group consisting of a tissue-preferred promoter, a tissue-specific promoter, an inducible promoter, and a constitutive promoter.

95. The method of embodiments 76 or 79, wherein the first promoter is selected from the group consisting of a tissue-preferred promoter, a tissue-specific promoter, an inducible promoter, and a constitutive promoter.

96. The method of embodiments 94 or 95, wherein the constitutive promoter is selected from the group consisting of a CAMV35S promoter, an Actin promoter, a Rab15 promoter, DAMV promoter and a Ubiquitin promoter.

97. The method of any one of embodiments 75-96, wherein the one or more guide nucleic acids comprises at least one guide RNA.

98. The method of any one of embodiments 75-97, wherein the first nucleic acid sequence, the second nucleic acid sequence, or both, are stably integrated into a genome of the first plant.

99. The method of any one of embodiments 75-98, wherein the plant cell is selected from the group consisting of corn, rice, sorghum, wheat, alfalfa, barley, millet, rye, sugarcane, cotton, soybean, canola, tomato, and potato.

100. The method of any one of embodiments 75-99, wherein the genome is selected from the group consisting of a nuclear genome, a mitochondrial genome, and a plastid genome.

101. The method of any one of embodiments 75-100, wherein repair of the double-stranded break generates at least one mutation in the target sequence as compared to a control plant of the same line or variety that lacks the first nucleic acid sequence or second nucleic acid sequence, optionally wherein the mutation results in the deletion, insertion or substitution of at least one nucleotide at or near the target sequence.

102. The method of embodiment 101, wherein the at least one mutation in the target sequence results in the deletion of one or more amino acids from a protein encoded by a gene of interest as compared to a wild-type protein.

103. The method of embodiment 101, wherein the at least one mutation in the target sequence results in the substitution of one or more amino acids within a protein encoded by a gene of interest as compared to a wild-type protein.

104. The method of embodiment 101, wherein the at least one mutation in the target sequence results in the introduction of a premature stop codon in a messenger RNA encoded by a gene of interest as compared to a wild-type messenger RNA.

105. The method of embodiment 101, wherein the at least one mutation in the target sequence comprises the deletion of one or more splice sites from a gene of interest.

106. The method of embodiments 76 or 78, wherein the sequence encoding the at least one guide nucleic acid is flanked by self-cleaving ribozymes.

107. The method of any one of embodiments 75-106, wherein the first plant is self-pollinated.

Having now generally described the disclosure, the same will be more readily understood through reference to the following examples that are provided by way of illustration, and are not intended to be limiting of the present disclosure, unless specified.

### EXAMPLES

### Example 1: Expression of Cas12a in Floral Cells/Tissue to Generate Germinal Mutations or Targeted Integration of Template DNA

Several Agrobacterium T-DNA vectors are generated to preferentially express Cas12a in corn or soy floral tissue. See Table 1. A control vector is generated to constitutively express Cas12a, where Cas12a is operably linked to a modified promoter comprising a DaMV promoter (SEQ ID NO:31) operably fused to an enhancer region from the Banana Streak Virus Strain Acuminata Vietnam (SEQ ID NO:43).

TABLE 1
Cassettes designed to express Cas12a preferentially,
or solely, in corn or soy meristematic tissue/cells.

| | Promoter for | | Promoter |
|---|---|---|---|
| Construct | LbCas12a | | SEQ |
| number | expression | Expression | ID NO |
| 1 | Corn AG1/ZAG1 | Male/Female spikelet | 1 |
| | (Zm00001d037737) | (C-Class gene) | |
| 2 | Corn ZAG-Like | Male/Female spikelet | 2 |
| | (Zm00001d051465) | (E-Class gene) | |
| 3 | Corn ZMM16 (pistulata) | Female spikelet | 3 |
| | (Zm00001d042618) | (B-Class gene) | |
| 4 | Corn ZAG5 | Male/Female spikelet | 4 |
| | (Zm0001d017614) | (D-Class gene) | |
| 5 | Corn ZAG2 | Female spikelet | 5 |
| | (Zm00001d041781) | (D-Class gene) | |
| 6 | Corn Ramosa 2 | Axillary meristem | 6 |
| | (Zm00001d039694) | | |
| 7 | Corn Ramosa 1 | Axillary meristem | 7 |
| | (Zm00001d020430) | | |
| 8 | Corn AGL16/MADS19 | Anther | 8 |
| | (Zm00001d016957) | (C-Class gene) | |
| 9 | Corn NAC119 | Axillary meristem | 9 |
| | (Zm00001d035266) | | |
| 10 | Corn Roxy/Male Sterile | Axillary meristem | 10 |
| | 22 (Zm0001d018802) | | |
| 11 | Arabidopsis pERL1 | Axillary meristem | 11 |
| | (US20180105819-0044) | | |
| 12 | Soy GmAP1-like- | Reproductive meristem | 12 |
| | 1(Glyma.01g064200) | (A-Class gene) | |
| 13 | Soy GmAP1-like- | Reproductive meristem | 13 |
| | 2(Glyma.02g121600) | (A-Class gene) | |
| 14 | Soy GmOsd2 | Sperm specific | 14 |
| | (Glyma.03g141800) | | |
| 15 | Soy GmAP3-like-1 | Floral meristem | 15 |
| | (Glyma.04g027200) | (B class gene) | |
| 16 | Soy GmERL4 | Axillary meristem | 16 |
| | (Glyma.04g056200) | | |
| 17 | Soy GmAP3-like-2 | Floral meristem | 47 |
| | (Glyma.06g027200) | (B-Class gene) | |
| 18 | Soy_GmERL3 | Axillary meristem | 18 |
| | (Glyma.06g056400) | | |
| 19 | Soy GmAP1-like-3 | Reproductive meristem | 19 |
| | (Glyma.08g269800) | (A -Class gene) | |
| 20 | Soy GmCYC3-1 | Floral meristem | 46 |
| | (Glyma.10g246200) | | |
| 21 | Soy GmPi-like | Floral meristem | 21 |
| | (Glyma.13g034100) | | |
| 22 | Sy GmTDF1 (MS6) | Tapetum specific | 22 |
| | (Glyma.13g066600) | | |
| 23 | Soy GmSUP-like | Floral meristem | 23 |
| | (Glyma.13g269400) | | |
| 24 | Soy GmERL2 | Axillary meristem | 24 |
| | (Glyma.14g098600) | | |
| 25 | Soy GmPi-like | Floral meristem | 25 |
| | (Glyma.14g155100) | | |
| 26 | Soy AP1-like | Reproductive meristem | 26 |
| | (Glyma.16g091300) | (A-Class gene) | |
| 27 | Soy GmERL1 | Axillary meristem | 27 |
| | (Glyma.17g226100) | | |
| 28 | Soy GmOsd1 | Sperm specific | 28 |
| | (Glyma.19g144400) | | |
| 29 | Soy CYC3- | Floral meristem | 29 |
| | 2(Glyma.20g148500) | | |
| 30 | Soy AGL42 | Reproductive meristem | 30 |
| | (Glyma.20g154200) | (D-Class gene) | |
| 31 | DaMV | Constitutive | 31 |

The plant codon optimized LbCas12a sequence (SEQ ID NO: 32) in these cassettes is flanked by NLS (Nuclear localization signal) sequences at the 5′ and 3′ ends and operably linked to a transcription terminator sequence. Each vector also contains an expression cassette encoding a Cas12a gRNA targeting a unique corn genomic site (ZmTS1) or a soy genomic site (GmTS1) and operably linked to a plant Pol III promoter; an expression cassette for a Gene of Interest (GOI) flanked by ZmTS1 or GmTS1 gRNA target sites; and an expression cassette for a selectable marker. Corn or soy embryos are transformed with the vectors described above by Agrobacterium-mediated transformation and R0 plants are regenerated from the transformed cells. DNA is extracted from leaf samples from R0 seedlings generated from each construct. The genomic target site is sequenced and analyzed for the presence, number, and types of mutations observed.

Several R0 lines comprising each transformed construct are grown to maturity and self-pollinated. Several R1 lines are subsequently selected and several seedlings per R1 line are germinated and screened for mutations within the target sites. The number and type of mutations produced using constructs comprising the floral tissue-specific promoters (constructs 1 to 30) are compared to the mutations observed in the transformed corn plants produced using control construct 31. Without being bound by any theory, it is anticipated that co-expression of Cas12a and its cognate gRNA in floral cells generates a double stranded break at the target site and subsequent imperfect DNA repair generates unique mutations in floral tissue. Restricting edits to developing reproductive structures can produce independent editing events in multiple locations within a single plant in those tissues that result in organs or cell types from which gametes are derived. These edited gametes can then pass the mutations onto the next generation. Thus, it is anticipated that a single R0 plant can produce many R1 offspring each with unique target site edits.

### Testing for Site Directed Integration (SDI) of T-DNA in Target Site in Plants Expressing Cas12a.

In addition to the Cas12a and gRNA expression cassette, each vector also contains an expression cassette for a gene of interest (GOI) that is flanked by the ZmTS1 or GmTS1 gRNA target sequences. Without being limited by any theory, expression of Cas12a and gRNA in floral tissues is expected to create double stranded breaks on both sides of the GOI cassette releasing it from the T-DNA. This released DNA can serve as a donor for targeted insertion at the genomic target site. When the CRISPR-Cas12a complex cuts the target site within the genome, the non-homologous end joining (NHEJ) DNA repair pathway could insert the donor GOI cassette into the genomic target site. This form of SDI is also known as trans-fragment targeting (TFT).

To test for SDI by TFT, flank PCR assays similar to those described in WO 2019/084148, which is incorporated herein by reference in its entirety, are used to identify putative targeted insertions. Primers are designed to PCR amplify the expected insertion flanking sequence. Four separate PCRs are performed: a left flank PCR and a right flank PCR for potential inserts that are positioned in the sense orientation, and a left flank PCR and a right flank PCR for inserts that are positioned in the antisense direction. R0 and R1 lines are screened to identify putative flank PCR positive plants which are further sequenced to confirm targeted insertion of the GOI cassette at the GmTS1 or ZmTS1 genomic sites.

### Example 2. Expression of gRNA in Floral Cells/Tissue with a Constitutively-Expressed Cas12a to Generate Germinal Mutations

Several constructs are generated to preferentially express a guide RNA (gRNA) complementary to a target site under the control of a Pol II promoter in floral cells. See Table 2. Following transcription, Pol II products are rapidly modified with a 5′ cap and poly-A tail and exported from the nucleus. These modifications and altered localization could prevent efficient use of gRNA. To optimize the gRNA availability and performance, self-cleaving ribozymes are incorporated into the gRNA cassette design. It has been reported that self-cleaving ribozymes facilitate cleavage/processing of the gRNA transcript from Pol II expressed transcripts to produce the precise guide molecule (see, for example, Wang et. al., J. of Integrative Plant Biol, 60:626-631 (2018)).

TABLE 2
Constructs designed to express a gRNA preferentially, or solely,
in soy or corn floral tissue. For all constructs noted below, the
gRNA cassette configuration is Promoter::ribozyme-gRNA-ribozyme

| Construct | Promoter for gRNA | Promoter |
|---|---|---|
| number | expression | SEQ ID NO |
| 32 | Corn AG1/ZAG1 | 1 |
| | (Zm00001d037737) | |
| 33 | Corn ZAG-Like | 2 |
| | (Zm00001d051465) | |
| 34 | Corn ZMM16 (pistulata) | 3 |
| | (Zm00001d042618) | |
| 35 | Corn ZAG5 | 4 |
| | (Zm0001d017614) | |
| 36 | Corn ZAG2 | 5 |
| | (Zm00001d041781) | |
| 37 | Corn Ramosa 2 | 6 |
| | (Zm00001d039694) | |
| 38 | Corn Ramosa 1 | 7 |
| | (Zm00001d020430) | |
| 39 | Corn AGL16/MADS19 | 8 |
| | (Zm00001d016957) | |
| 40 | Corn NAC119 | 9 |
| | (Zm00001d035266) | |
| 41 | Corn Roxy/Male Sterile | 10 |
| | 22 (Zm0001d018802) | |
| 42 | Arabidopsis pERL1 | 11 |
| | (US20180105819-0044) | |
| 43 | Soy AP1-like | 12 |
| | (Glyma.01g064200) | |
| 44 | Soy AP1-like | 13 |
| | (Glyma.02g121600) | |
| 45 | Soy GmOsd2 | 14 |
| | (Glyma.03g141800) | |
| 46 | Soy GmAP3-like | 15 |
| | (Glyma.04g027200) | |
| 47 | Soy GmERL4 | 16 |
| | (Glyma.04g056200) | |
| 48 | Soy GmAP3-like | 47 |
| | (Glyma.06g027200) | |
| 49 | Soy_GmERL3 | 18 |
| | (Glyma.06g056400) | |
| 50 | Soy AP1-like | 19 |
| | (Glyma.08g269800) | |
| 51 | Soy CYC3 | 46 |
| | (Glyma.10g246200) | |
| 52 | Soy GmPi-like | 21 |
| | (Glyma.13g034100) | |
| 53 | Sy GmTDF1 (MS6) | 22 |
| | (Glyma.13g066600) | |
| 54 | Soy GmSUP-like | 23 |
| | (Glyma.13g269400) | |
| 55 | Soy GmERL2 | 24 |
| | (Glyma.14g098600) | |
| 56 | Soy GmPi-like | 25 |
| | (Glyma.14g155100) | |
| 57 | Soy AP1-like | 26 |
| | (Glyma.16g091300) | |
| 58 | Soy GmERL1 | 27 |
| | (Glyma.17g226100) | |
| 59 | Soy GmOsd1 | 28 |
| | (Glyma.19g144400) | |
| 60 | Soy CYC3 | 29 |
| | (Glyma.20g148500) | |
| 61 | Soy AGL42 | 30 |
| | (Glyma.20g154200) | |
| 62 (control) | DaMV | 31 |

The constructs described in Table 2 are stably introduced into corn or soy cells using transformation methods routinely used in the art. Additionally, a construct (“Cas12a construct”) comprising a plant codon optimized nucleic acid sequence encoding a Cas12a protein flanked by NLS sequences at the 5′ and 3′ ends and under the control of a ubiquitous promoter (e.g.: ZmUbqM1 promoter (SEQ ID NO: 35) or Medicago truncatula Ubq2 promoter (SEQ ID NO:44)) is co-introduced with each construct provided in Table 2. The resulting transformed cells comprise one of constructs 32 to 62, as well as the Cas12a construct. Plants are regenerated from the transformed cells, grown to maturity and pollinated. Seed resulting from the pollination is screened for mutations in the target site, and the number and type of mutations produced using constructs 32-61 are compared to the mutations in transformed plants produced using control construct 62. Selective expression of gRNA is expected to generate one or more unique mutations in floral tissue.

### Example 3. Expression of Cas12a and gRNA as a Single Transcript in Floral Cells

Several constructs are generated to preferentially express LbCas12a and a guide RNA (gRNA) complementary to a target site as a single transcript in. See Table 3.

TABLE 3
Constructs designed to express a LbCas12a and a gRNA flanked
by self-cleaving ribozymes in a single transcript in
corn or soy floral cells/tissue. The cassette configuration
is Promoter::LbCas12a-ribozyme-gRNA-ribozyme

| | Promoter to drive | |
|---|---|---|
| Construct | Cas12a and g RNA | Promoter |
| number | expression | SEQ ID NO |
| 63 | Corn AG1/ZAG1 | 1 |
| | (Zm00001d037737) | |
| 64 | Corn ZAG-Like | 2 |
| | (Zm00001d051465) | |
| 65 | Corn ZMM16 (pistulata) | 3 |
| | (Zm00001d042618) | |
| 66 | Corn ZAG5 | 4 |
| | (Zm0001d017614) | |
| 67 | Corn ZAG2 | 5 |
| | (Zm00001d041781) | |
| 68 | Corn Ramosa 2 | 6 |
| | (Zm00001d039694) | |
| 69 | Corn Ramosa 1 | 7 |
| | (Zm00001d020430) | |
| 70 | Corn AGL16/MADS19 | 8 |
| | (Zm00001d016957) | |
| 71 | Corn NAC119 | 9 |
| | (Zm00001d035266) | |
| 72 | Corn Roxy/Male Sterile | 10 |
| | 22 (Zm0001d018802) | |
| 73 | Arabidopsis pERL1 | 11 |
| | (US20180105819-0044) | |
| 74 | Soy AP1-like | 12 |
| | (Glyma.01g064200) | |
| 75 | Soy AP1-like | 13 |
| | (Glyma.02g121600) | |
| 76 | Soy GmOsd2 | 14 |
| | (Glyma.03g141800) | |
| 77 | Soy GmAP3-like | 15 |
| | (Glyma.04g027200) | |
| 78 | Soy GmERL4 | 16 |
| | (Glyma.04g056200) | |
| 79 | Soy GmAP3-like | 47 |
| | (Glyma.06g027200) | |
| 80 | Soy_GmERL3 | 18 |
| | (Glyma.06g056400) | |
| 81 | Soy AP1-like | 19 |
| | (Glyma.08g269800) | |
| 82 | Soy CYC3 | 46 |
| | (Glyma.10g246200) | |
| 83 | Soy GmPi-like | 21 |
| | (Glyma.13g034100) | |
| 84 | Sy GmTDF1 (MS6) | 22 |
| | (Glyma.13g066600) | |
| 85 | Soy GmSUP-like | 23 |
| | (Glyma.13g269400) | |
| 86 | Soy GmERL2 | 24 |
| | (Glyma.14g098600) | |
| 87 | Soy GmPi-like | 25 |
| | (Glyma.14g155100) | |
| 88 | Soy AP1-like | 26 |
| | (Glyma.16g091300) | |
| 89 | Soy GmERL1 | 27 |
| | (Glyma.17g226100) | |
| 90 | Soy GmOsd1 | 28 |
| | (Glyma.19g144400) | |
| 91 | Soy CYC3 | 29 |
| | (Glyma.20g148500) | |
| 92 | Soy AGL42 | 30 |
| | (Glyma.20g154200) | |
| 93 (control) | DaMV | 31 |

Each construct described in Table 3 is stably introduced into corn or soy cells using biolistic transformation methods or Agrobacterium-mediated transformation methods routinely used in the art. The resulting transformed corn cells comprise one of constructs 63-93. Plants are regenerated from the transformed cells and grown to maturity. The LbCas12a and gRNA are transcribed as part of a single transcript in floral cells where the promoter expresses. Subsequently, ribozyme mediated cleavage occurs releasing the gRNA segments. LbCas12a protein transcribed from the transcript forms ribonucleoproteins (RNPs) with the gRNAs. The RNPs generate a double-stranded break at the target site and subsequent repair will generate one or more unique mutations in each floral cell. Mature plants are pollinated and seeds resulting from the pollination are screened for mutations in the target site and the number and type of mutations produced using constructs 63-92 is compared to the transformed corn plants produced using control construct 93.

### Example 4. Generating Mutations Via Crossing

Transgenic corn or soy plants comprising one of the LbCas12a cassettes described in Table 1 are generated and grown to flowering stage. An additional transgenic corn or soy plant comprising the gRNA cassette described in Example 1 is also generated and grown to flowering stage. The LbCas12a comprising plants are crossed with the plant comprising the gRNA construct, generating progeny plants comprising Cas12a and the gRNA being expressed in the floral tissue.

Alternatively, transgenic corn or soy plants comprising one of Constructs (see Example 2, Table 2) are generated and grown to flowering stage. An additional transgenic corn or soy plant comprising the Cas12a construct of Example 2 is also generated and grown to flowering stage. The transgenic plants comprising one of Constructs are crossed with the plant comprising the Cas12a construct, generating progeny plants comprising Cas12a and the gRNA being expressed in the floral tissue.

Without being limited by any theory, co-expression of Cas12a and the gRNA in floral tissue will generate a double-stranded break within the target site, thereby generating a unique mutation in each cell where both components of the CRISPR system are expressed. The plants are crossed or self-pollinated and the resulting progeny are screened to identify mutations in the target site.

### Example 5: TALE Induced Tissue/Cell Specific Expression of LbCas12a

A potential downside of tissue/cell preferred promoters is that they tend to not be robustly expressed. This example describes constructs that have been generated to overcome this limitation and induce robust expression of a transcribable polynucleotide, such as Cas12a, in a tissue/cell preferred manner.

Several constructs are generated for robust, TALE-induced Cas12a expression preferentially in floral cells/tissue. Transcription Activator-Like Effectors (TALEs) are transcription factors that comprise a C terminal activation domain and can activate/increase the expression of an operably linked transcribable polynucleotide once TALEs bind to the TALE binding site at or near the promoter. It has previously been shown that TALE proteins can induce high expression of a gene operably linked to a TALE binding site, and that expression can be modulated depending on how many of the TALE binding sites are present in the regulatory region. Constructs are generated comprising a plant codon optimized LbCas12a coding sequence flanked by NLS sequences at the 5′ and 3′ ends and operably linked to a transcription terminator sequence and a minimal 35S(-46) promoter with one, three, or six TALE binding sites. Expression constructs are also generated comprising a TALE coding sequence operably linked to a promoter that preferentially or solely expresses in floral tissue/cells. Non-limiting examples of promoters and regulatory sequences to drive preferential cell expression are provided in Table 1. An expression cassette comprising a TALE coding sequence operably linked to a constitutive Ubiquitin promoter is generated as a control. Corn or soy embryos are transformed with a vector(s) comprising the expression cassettes as described above and an expression cassette encoding a Cas12a gRNA complementary to a corn genomic target site (ZmTS1) or Soy genomic target site (GmTS1) under the control of a plant Pol III promoter and an expression cassette for a selectable marker by agrobacterium-mediated transformation and R0 plants are generated from the transformed cells. Several R0 lines from each transformed construct are grown to maturity and are pollinated. Several R1 lines are selected, seedlings are germinated and screened for LbCas12a induced edits in the target site and the editing rates are calculated. It is anticipated that when plants comprising the Cas12a, gRNA and TALE expression vectors described above reach the reproductive stage, TALE expressed preferentially in the floral tissue/cells will bind to TALE protein binding sites upstream of the 35S(-46)::Lb.Cas12a and induce robust expression of the nuclease preferentially in floral cells. Expression of LbCas12a and gRNA is expected to lead to mutations within the target site. The R1 plants generated from the transformed R0 lines are expected to exhibit a significant number of unique mutations at the target site.

### Example 6. Preferential Expression of Cas12a in Floral Tissue/Cell to Generate Germinal Mutations

This example describes the use of Arabidopsis thaliana meristematic tissue-preferred promoter AtERL1 to drive the expression of Cas12a expression so as to generate diverse mutations at R0 generation and beyond. As shown in Table 4, two Agrobacterium T-DNA constructs were generated. Each construct comprised an LbCas12a nuclease cassette, a gRNA array cassette, and a selectable marker cassette. The vectors are similar in design except that in Construct 94 the LbCas12a cassette is driven by Arabidopsis ERL1 (SEQ ID NO: 11) a meristematic tissue-preferred promoter while in the control construct 95, LbCas12a is driven by strong constitutive promoter DaMV.

TABLE 4
Cassettes designed for constitutive expression and preferential
expression of Cas12a, in soy axillary meristematic tissue.

| | Promoter::LbCas12a::Mt | | Promoter |
|---|---|---|---|
| Construct | term | Expression | SEQ ID NO. |
| 94 | AtERL1::LbCas12a | Axillary | 11 |
| | | meristem | |
| 95 | DaMV::LbCas12a | Constitutive | 31 |

The plant codon optimized LbCas12a sequence (SEQ ID NO:36) in these cassettes is flanked by NLS sequences at the 5′ and 3′ ends (SEQ ID: 33 and SEQ ID: 34) and operably linked to a transcription terminator sequence from a Medicago truncatula gene (SEQ ID NO: 37). The gRNA array expression cassette comprises a Pol III promoter operably linked to four guide RNAs (see Table 5), each targeting a 27 nucleotide sequence within an 864 nucleotide E1 genic sequence in the Glycine maxgenome (SEQ ID NO: 38). The T-DNA vector also comprises an expression cassette for a selectable marker conferring resistance to the antibiotic spectinomycin. Soy A3555 cultivar embryos were transformed with the vectors described above by Agrobacterium-mediated transformation and R0 plants were regenerated from the transformed soy cells.

TABLE 5
gRNA target site coordinates on 864 nucleotide GmE1 genic site

| | Target site | | |
|---|---|---|---|
| | coordinates on | | |
| Guide | GmE1 | Target site sequence. | |
| RNA | (SEQ ID NO 38) | PAM shown in Upper case | SEQ ID NO |
| gRNA1 | 275..301 | TTTAggacatcaaggagaagattctgc | 39 |
| gRNA2 | 342..368 | TTTCaacaacactgaagctttacgatg | 40 |
| gRNA3 | 406..432 | TTTGggaatcctaagtagactcttgct | 41 |
| gRNA4 | 522..548 | TTTGggacatggacaccaaatccatgc | 41 |

DNA is extracted from leaf samples from 20 R0 seedlings for each of Construct 94 and Construct 95. The GmE1 site is sequenced and analyzed for the presence of targeted mutations. Co-expression of Cas12a and its cognate gRNA is expected to generate a double stranded break at the target sites and subsequent imperfect DNA repair generates unique mutations. Six target site edits were identified in plants carrying the AtEr11:LbCas12a (Construct 94) and 15 target site edits were observed in plants transformed with the DaMV:LbCas12a construct (Construct 95) (see table 6). A low mutation rate from Construct 94 in the newly transformed (or R0) plants is expected since AtERL1 is predicted to be a weak promoter that is preferentially expressed in axillary meristematic tissue.

TABLE 6
Cutting efficacy of LbCas12a driven by
different promoters in the R0 plants

| | | Total | Edits/ |
|---|---|---|---|
| | R0 plants | number | sequenced |
| Construct | sequenced | of edits | plants |
| (AtErl1:LbCas12a) | 20 | 6 | 0.30 |
| (DaMV:LbCas12a) | 23 | 15 | 0.65 |

Several R0 lines from each transformed construct were grown to maturity and at least one ear from each transformed soyplant was self-pollinated. Several hundred R1 lines were selected, germinated and screened for mutations. As shown in Table 7, R1 progenies derived from AtERL1:LbCas12a transgenic events carried many more new mutations when compared to the events expressing Cas12a from the constitutive promoter DaMV.

TABLE 7
Number of newly derived edits identified in R1 progenies

| | | Total | | Total new | New edits |
|---|---|---|---|---|---|
| | R1 | edits in | Edits/ | edits | identified |
| | plants | R1 | R1 | identified | in R1 gen/ |
| Construct | sequenced | plants | plant | in R1 | R1 plant |
| AtERL1:LbCas12a | 647 | 421 | 0.65 | 353 | 0.55 |
| DaMV:LbCas12a | 418 | 341 | 0.82 | 17 | 0.04 |

Sequence analysis of the edits revealed that the newly generated mutations detected in R1 plants of AtERL1:LbCas12a transgenic events were very diverse. A broad spectrum of mutations identified from about 40 R1 plants of each event (see Table 8). Furthermore, several edits were identified in R1 plants where the parental R0 plant was not edited.

TABLE 8
Edits in R0 parent and R1 progeny. Number of newly derived edits,
differed from R0 edits, identified in R1 progenies. Each edit is
annotated based on the location of the edit relative to the target
GmES1, the type of edit and the number of base pairs edited. Δ
indicates deletion, ‘s’ indicates substitution, ‘I’
indicates insertion. The number of R1 plants having the specific
mutation are indicated in parenthesis. For example, 525Δ24 (2)
refers to a 24 nucleotide deletion starting at position 525 within
the GmES1 sequence, and this mutation was detected in two R1 plants.

| | | | |
|---|---|---|---|
| | | R1 | |
| | R0 | plant | New edits identified |
| Pedigree | edit | # | in R1 plants |

DaMV::LbCas12a

| | | | |
|---|---|---|---|
| GM_S22596775 | 368Δ190 | 38 | None |
| GM_S22596804 | 290Δ97 | 40 | None |
| GM_S22596809 | 428Δ2 | 35 | None |
| GM_S22596831 | 450Δ100 | 37 | None |
| GM_S22596838 | 388Δ161 | 38 | None |
| GM_S22596840 | 540Δ9 | 39 | None |
| GM_S22596846 | 545Δ5 | 40 | 363Δ198 (1); |
| | | | 507Δ44, (1); |
| | | | 519Δ34, (2); |
| | | | 541Δ6, (1); |
| | | | 542Δ13, (1); |
| | | | 542Δ9, (1); |
| | | | 543Δ6, (1); |
| | | | 547Δ4, (1); |
| GM_S22596850 | 543Δ8 | 37 | None |
| GM_S22596855 | 541Δ10 | 39 | 535Δ13, (1); |
| | | | 542Δ13, (1); |
| | | | 543Δ8, (1); |
| GM_S22596863 | 547Δ4 | 37 | 471Δ77, (1); |
| | | | 497Δ57, (1); |
| | | | 535Δ76, (1); |
| | | | 542Δ13, (1); |
| GM_S22596873 | 295Δ9 | 35 | 545Δ1; (1) |

AtERL1::LbCas12a

| | | | |
|---|---|---|---|
| GM_S22596891 | NA | 40 | 323Δ235, (1); |
| | | | 480Δ67, (1); |
| | | | 507Δ77, (1); |
| | | | 508Δ47, (1); |
| | | | 515Δ31, (1); |
| | | | 532s2/534Δ15*, (1); |
| | | | 538Δ10, (1); |
| | | | 539s1/540Δ8*, (1); |
| | | | 541Δ10, (3); |
| | | | 541Δ6, (2); |
| | | | 541Δ8, (1); |
| | | | 542Δ6, (1); |
| | | | 542Δ7, (1); |
| | | | 543Δ8, (2); |
| | | | 544Δ4, (1); |
| | | | 547Δ4, (1); |
| GM_S22596892 | NA | 39 | 548Δ2(1) |
| GM_S22596893 | 541Δ10 | 9 | None |
| GM_S22596895 | 541Δ10 | 24 | 293Δ9, (1); |
| | | | 295Δ7, (1); |
| | | | 303Δ247, (1); |
| | | | 379Δ170, (1); |
| | | | 390Δ4, (1); |
| | | | 392Δ6, (1); |
| | | | 472Δ78, (1); |
| | | | 478Δ73, (1); |
| | | | 541Δ4, (2); |
| | | | 541Δ6, (1); |
| | | | 542Δ7, (2); |
| | | | 543Δ6, (2); |
| | | | 543Δ8, (1); |
| | | | 544Δ4, (1); |
| | | | 556Δ55, (1); |
| GM_S22596896 | NA | 37 | 428Δ2, (1); |
| | | | 451Δ112, (1); |
| | | | 456Δ95, (1); |
| | | | 520Δ84, (1); |
| | | | 529Δ14, (1); |
| | | | 536Δ28, (1); |
| | | | 541Δ10, (1); |
| | | | 541Δ4, (1); |
| | | | 542s1/543Δ15*, (1); |
| | | | 542Δ9, (2); |
| | | | 543Δ6, (1); |
| | | | 543Δ7, (1); |
| | | | 543Δ8, (1); |
| | | | 544Δ4, (1); |
| GM_S22596898 | NA | 40 | 538Δ9, (1); |
| | | | 541Δ7, (1); |
| | | | 542Δ13, (1); |
| GM_S22596899 | NA | 40 | 283Δ88, (1); |
| | | | 302Δ248, (1); |
| | | | 359Δ13, (1); |
| | | | 433Δ116, (1); |
| | | | 537Δ12, (1); |
| | | | 541Δ10, (3); |
| | | | 541Δ7, (1); |
| | | | 541Δ8, (1); |
| | | | 542Δ9, (1); |
| | | | 545Δ1, (1); |
| | | | 547Δ4, (1); |
| GM_S22596900 | NA | 38 | 290Δ12, (1); |
| | | | 291Δ8, (1); |
| | | | 369Δ179, (1); |
| | | | 373Δ176, (1); |
| | | | 405Δ26, (1); |
| | | | 428Δ2, (2); |
| | | | 469Δ80, (1); |
| | | | 528s1/529Δ16*, (1); |
| | | | 541Δ10, (4); |
| | | | 541Δ4, (1); |
| | | | 541Δ6, (1); |
| | | | 541Δ8, (1); |
| | | | 542Δ1, (1); |
| | | | 542Δ13, (5); |
| | | | 542Δ9, (5); |
| | | | 543i1, (1); |
| | | | 543Δ8, (4); |
| | | | 544Δ4, (1); |
| | | | 545Δ5, (2); |
| GM_S22596901 | NA | 39 | 541Δ10, (1); |
| | | | 542Δ7, (1); |
| GM_S22596902 | NA | 39 | 353Δ199, (1); |
| | | | 541Δ10, (2); |
| | | | 542Δ1, (1); |
| | | | 542Δ9, (2); |
| | | | 542Δ13, (2); |
| GM_S22596904 | 542Δ9 | 69 | 360Δ10, (1); |
| | | | 428Δ2, (1); |
| | | | 539Δ9, (1); |
| | | | 541Δ10, (3); |
| | | | 541Δ4, (1); |
| | | | 543Δ13, (1); |
| | | | 543Δ8, (1); |
| | | | 544Δ4, (1); |
| GM_S22596905 | NA | 76 | 364Δ5, (1); |
| | | | 393Δ158, (1); |
| | | | 425Δ6, (1); |
| | | | 428Δ2, (1); |
| | | | 480Δ67, 10; |
| | | | 497Δ49, (1); |
| | | | 541Δ10, (2); |
| | | | 541Δ6, (1); |
| | | | 542Δ9, (4); |
| | | | 543Δ8, (1); |
| | | | 544Δ4, (2); |
| | | | 547Δ4, (1); |
| GM_S22596907 | NA | 40 | 348Δ199, (1); |
| | | | 428Δ2, (1); |
| | | | 541Δ4, (1); |
| | | | 541Δ6, (2); |
| | | | 542Δ9, (3); |
| | | | 543Δ1, (1); |
| | | | 543Δ12, (1); |
| | | | 543Δ6, (1); |
| | | | 543Δ8, (1); |
| | | | 545Δ5, (1); |
| | | | 547Δ4, (2); |
| | | | 548Δ45, (1); |
| | | | 549i1, (1); |
| GM_S22596910 | 369Δ3 | 78 | 285Δ19, (1); |
| | | | 296Δ6, (1); |
| | | | 351Δ196, (1); |
| | | | 362Δ186, (1); |
| | | | 367Δ69, (1); |
| | | | 416Δ17, (1); |
| | | | 423Δ10, (1); |
| | | | 424s1/425Δ9*, (2); |
| | | | 428Δ2, (4); |
| | | | 428Δ6, (3); |
| | | | 479Δ71, (1); |
| | | | 509Δ39, (1); |
| | | | 509Δ49, (1); |
| | | | 515Δ31, (1); |
| | | | 517Δ32, (1); |
| | | | 525Δ24, (1); |
| | | | 528s1, (1); |
| | | | 537Δ11, (1); |
| | | | 538Δ15, (2); |
| | | | 541Δ10, (4); |
| | | | 541Δ4, (5); |
| | | | 541Δ6, (4); |
| | | | 541Δ7, (1); |
| | | | 542s1/543Δ7*, (1); |
| | | | 542Δ1, (1); |
| | | | 542Δ10, (1); |
| | | | 542Δ13, (2); |
| | | | 542Δ7, (1); |
| | | | 542Δ9, (8); |
| | | | 543Δ12, (1); |
| | | | 543Δ8, (4); |
| | | | 544Δ4, (4); |
| | | | 545Δ1, (1); |
| | | | 545Δ5, (2); |
| | | | 547Δ4, (5); |

*indicates biallelic mutations.
NA—no edits were detected.

Closer analysis of the segregation patter of edits/mutations reveals that unlike what occurred in the R1 progenies of DAMV:LbCas12a, the segregation pattern of edits identified in R0 events of AtERL1:LbCas12a may or may not follow the classic Mendelian pattern in the R1 progenies depending on when the mutation was introduced to the R0 plant (Table 9). For events with atypical segregation patterns, without being limited by any theory, mutations are most likely arising from meristematic tissues after regenerated R0 plant. Thus, the R1 seeds is a mixture of seeds from a chimeric R0 plant.

TABLE 9
R1 segregation pattern of edits detected in R0 plants

| | | Segregation in R1 | | | | | |
|---|---|---|---|---|---|---|---|
| | Edit | progenies | | | | Expected | χ² |
| Event | in R0 | Total | Homo | Het | Null | segregation | (P0.05 = 5.99) |

DAMV:LbCas12a

| | | | | | | | |
|---|---|---|---|---|---|---|---|
| GM_S22596804 | 290Δ97 | 40 | 8 | 19 | 13 | 1:2:1 | 1.35 |
| GM_S22596809 | 428Δ2 | 35 | 8 | 18 | 9 | 1:2:1 | 0.09 |
| GM_S22596831 | 450Δ100 | 37 | 11 | 14 | 12 | 1:2:1 | 2.24 |
| GM_S22596838 | 388Δ161 | 38 | 8 | 16 | 14 | 1:2:1 | 2.84 |
| GM_S22596840 | 540Δ9 | 39 | 11 | 17 | 11 | 1:2:1 | 0.64 |
| GM_S22596846 | 545Δ5 | 40 | 3 | 26 | 11 | 1:2:1 | 6.80 |
| GM_S22596850 | 543Δ8 | 37 | 12 | 15 | 10 | 1:2:1 | 1.54 |
| GM_S22596855 | 541Δ10 | 39 | 14 | 19 | 6 | 1:2:1 | 3.31 |
| GM_S22596863 | 547Δ4 | 37 | 9 | 13 | 12 | 1:2:1 | 2.46 |

AtErl1:LbCas12a

| | | | | | | | |
|---|---|---|---|---|---|---|---|
| GM_S22596891 | NA | 40 | — | — | — | — | — |
| GM_S22596892 | NA | 39 | — | — | — | — | — |
| GM_S22596893 | 541Δ10 | 9 | 0 | 2 | 7 | 1:2:1 | 13.67 |
| GM_S22596895 | 541Δ10 | 24 | 9 | 11 | 3 | 1:2:1 | 3.08 |
| GM_S22596896 | NA | 37 | — | — | — | — | — |
| GM_S22596898 | NA | 40 | — | — | — | — | — |
| GM_S22596899 | NA | 40 | — | — | — | — | — |
| GM_S22596900 | NA | 38 | — | — | — | — | — |
| GM_S22596901 | NA | 39 | — | — | — | — | — |
| GM_S22596902 | NA | 39 | — | — | — | — | — |
| GM_S22596904 | 542Δ9 | 69 | 13 | 38 | 18 | 1:2:1 | 1.43 |
| GM_S22596905 | NA | 76 | — | — | — | — | — |
| GM_S22596907 | NA | 40 | — | — | — | — | — |
| GM_S22596910 | 369Δ3 | 78 | 12 | 20 | 46 | 1:2:1 | 48.15 |

Taken together, the data shows that reproductive editing can be achieved when LbCas12a is expressed under the control of the axillary meristematic promoter AtERL1. Additionally, the data demonstrates that a single R0 plant can produce many R1 offspring each with unique target site edits. This suggests that this promoter can be used to drive the expression of nucleases so as to increase the frequency of unique edits produced per transformed plants.

### Example 7: Preferential Expression of Cas12a in Floral Tissue/Cell to Generate Germinal Mutations

This example describes the use of Glycine max meristematic tissue-preferred promoters from the GmAP1-like, GmCYC3-1, GmAP3-like-1 and GmERL1-like genes to drive the expression of Cas12a expression so as to generate diverse mutations at the R0 generation and beyond. GmERL1-like is the soy homolog of the AtERL1 (SEQ ID NO:11) gene. As shown in Table 10, 5 Agrobacterium T-DNA constructs were generated. Each construct comprised an LbCas12a nuclease cassette, a gRNA array cassette, and a selectable marker cassette. The vectors are similar in design except that in construct 91 the LbCas12a cassette is driven by the strong constitutive promoter derived from Medicago truncatula Ubq2 gene (SEQ ID NO:44), while in the others LbCas12a expression is driven by various meristem-preferred promoters derived from soy (Glycine max).

TABLE 10
Cassettes designed for constitutive expression and tissue-preferred
expression of Cas12a in soybean meristematic tissue.

| | Promoter::LbCas12a::Mt | | Promoter |
|---|---|---|---|
| Construct | term. | Expression | seq used |
| 96 | MtUbq2::LbCas12a | Constitutive | 44 |
| 97 | GmAP1-like-1- | Reproductive | 45 |
| | var::LbCas12a | meristems | |
| 98 | GmCYC3-1::LbCas12a | Floral meristems | 46 |
| 99 | GmAP3-like-1::LbCas12a | Floral meristems | 47 |
| 100 | GmERL1-var::LbCas12a | Axillary | 48 |
| | | meristem | |

Var = variant.

In Construct 97, the LbCas12a expression is driven by a variant of the soy GmAP1-like-1 promoter (SEQ ID NO: 12) and is disclosed as GmAP1-like-1-var (SEQ ID NO: 45). GmAP1-like-1-var (SEQ ID NO: 45) comprises a 7 nucleotide 5′ extension and 371 nucleotide 3′ extension as compared to GmAP1-like-1 (SEQ ID NO: 12). In Construct 100, the LbCas12a expression is driven by a variant of the soy GmERL1promoter (SEQ ID NO: 27) and is disclosed as GmERL1-var (SEQ ID NO: 48). GmERL1-var (SEQ ID NO: 48) comprises a 261nucleotide 5′ deletion and 972 nucleotide 3′ extension as compared to GmERL1 (SEQ ID NO: 27).

The plant codon optimized LbCas12a sequence (SEQ ID NO: 32) in these cassettes is flanked by NLS sequences at the 5′ and 3′ ends (SEQ ID: 33 and SEQ ID: 34) and operably linked to a transcription terminator sequence from a Medicago truncatula gene (SEQ ID NO:50). The gRNA expression cassette comprises a Pol III promoter operably linked to one guide RNA (see Table 11), targeting a 27-nucleotide sequence within the 1542 nucleotide Tawny coding sequence in the Glycine max genome (SEQ ID NO: 51). The T-DNA vector also comprises an expression cassette for a selectable marker conferring resistance to antibiotics spectinomycin and streptomycin. Soy A3555 cultivar embryos were transformed with the vectors described above by Agrobacterium-mediated transformation and R0 plants were regenerated from the transformed soy cells.

TABLE 11
gRNA target site coordinates on 1542 nucleotide GmTawny coding sequence

| | Target site | | |
|---|---|---|---|
| | coordinates on | | |
| Guide | GmTawny CDS | Target site sequence. | |
| RNA | (SEQ ID NO 50) | PAM shown in Upper case | SEQ ID NO |
| gRNA1 | 1055..1077 | TTTCcttgataacagcttgtaagtatg | 52 |

DNA is extracted from leaf samples from 20 R0 seedlings for each of Construct 96-100. The GmTawny site is sequenced and analyzed for the presence of targeted mutations. Co-expression of Cas12a and its cognate gRNA is expected to generate a double stranded break at the target site and subsequent imperfect DNA repair generates unique mutations. Ten target site edits were identified in plants carrying the MtUbq2::LbCas12a construct (Construct 1) (see table 12). Total edits in transformations with tissue specific expression vary but are in the same magnitude as those for the constitutive transformation. Mutation rates are governed both by tissue specificity and differences in expression magnitude.

TABLE 12
Cutting efficacy of LbCas12a driven by
different promoters in the R0 plants

| | | R0 | Total | Edits/ |
|---|---|---|---|---|
| Con- | Promoter::LbCas12a::Mt | plants | number | sequenced |
| struct | term | sequenced | of edits | plants |
| 96 | MtUbq2::LbCas12a | 11 | 10 | 0.91 |
| 97 | GmAP1-like-1-var::LbCas12a | 13 | 12 | 0.92 |
| 98 | GmCYC3-1::LbCas12a | 12 | 9 | 0.75 |
| 99 | GmAP3-like-1::LbCas12a | 10 | 5 | 0.50 |
| 100 | GmERL1-var::LbCas12a | 11 | 11 | 1.00 |

Several R0 lines from each transformed construct were grown to maturity and seed was harvested. Several hundred R1 progeny seed were selected, germinated and screened for mutations. As shown in Table 13, R1 progenies derived from GmAP3like:LbCas12a and GmERL1like:LbCas12a transgenic events carried a higher frequency of new mutations when compared to the events expressing Cas12a from the constitutive promoter Ubq2.

TABLE 13
Number of newly derived edits identified in R1 progenies edits

| | | | Total | | Total | New edits |
|---|---|---|---|---|---|---|
| | | | edits | | new edits | identified |
| | | R1 plants | in R1 | Edits/R1 | identified | in R1 gen/R1 |
| Construct | Promoter::LbCas12a::Mt term | sequenced | plants | plant | in R1 | plant |
| 96 | MtUbq2::LbCas12a | 433 | 345 | 0.80 | 14 | 0.03 |
| 97 | GmAP1-like-1-var::LbCas12a | 504 | 461 | 0.92 | 8 | 0.02 |
| 98 | GmCYC3-1::LbCas12a | 341 | 192 | 0.56 | 2 | 0.01 |
| 99 | GmAP3-like-1::LbCas12a | 99 | 60 | 0.61 | 18 | 0.18 |
| 100 | GmERL1-var::LbCas12a | 262 | 236 | 0.90 | 22 | 0.08 |

Sequence analysis of the edits revealed that the newly generated mutations detected in R1 plants of GmAP3-like-1::LbCas12a and GmERL1-var::LbCas12a transgenic events were very diverse. A broad spectrum of mutations identified from about 40 R1 plants of each event (see Table 14). Furthermore, several edits were identified in R1 plants where the parental R0 plant was not edited.

Table 14. Edits in R0 parent and R1 progeny. Number of newly derived edits, differing from R0 edits identified in R1 progenies. Each edit is annotated based on the location (S) of the edit relative to the gRNA target sequence (SEQ ID NO: 50), the type of edit and the number of base pairs edited. Δ indicates deletion, ‘s’ indicates substitution, ‘I’ indicates insertion. The number of R1 plants having the specific mutation are indicated in parenthesis. For example, S6Δ4 (2) refers to a 4 nucleotide deletion starting at position 6 within the gRNA target site, and this mutation was detected in two R1 plants. S8s1 refers to a single basepair substitution at position 8 within the gRNA target site. S-4Δ13 refers to a 13 nucleotide deletion starting 4 nucleotides upstream of the target site. NA—no edits were detected.

TABLE 14
Edits in R0 parent and R1 progeny. Number of newly derived edits, differing from R0 edits
identified in R1 progenies. Each edit is annotated based on the location (S) of the edit
relative to the gRNA target sequence (SEQ ID NO: 50), the type of edit and the number
of base pairs edited. Δ indicates deletion, ‘s’ indicates substitution, ‘I’
indicates insertion. The number of R1 plants having the specific mutation are indicated
in parenthesis. For example, S6Δ4 (2) refers to a 4 nucleotide deletion starting at
position 6 within the gRNA target site, and this mutation was detected in two R1 plants.
S8s1 refers to a single basepair substitution at position 8 within the gRNA target site.
S-4Δ13 refers to a 13 nucleotide deletion starting 4 nucleotides upstream of the target site.

| | | | R1 | Type and number of | |
|---|---|---|---|---|---|
| | | | plant | new edits identified | Unique |
| Expression:Nuclease | Event | R0 edit | # | in R1 plants | R1 edits |
| MtUbq2::LbCas12a | GM_S22873169 | S-4Δ13; | 50 | None | 0 |
| | | S8s1 | | | |
| | GM_S22873171 | NA | 47 | None | 0 |
| | GM_S22873173 | S-1Δ15; | 27 | S2Δ7 (1) | 1 |
| | | S6Δ4 | | | |
| | GM_S22873177 | S6Δ4 | 46 | S2Δ7 (27) | 1 |
| | GM_S22873179 | S6Δ4 | 44 | None | 0 |
| | GM_S22873186 | S6Δ4 | 44 | S5Δ3 (8), | 8 |
| | | | | S-2Δ8 (1), | |
| | | | | S1Δ9 (1), | |
| | | | | S2Δ4 (2), | |
| | | | | S-1Δ8 (1), | |
| | | | | S3Δ8 (2), | |
| | | | | S3Δ19 (1), | |
| | | | | S4Δ5 (1) | |
| | GM_S22873196 | S-3Δ15; | 35 | None | 0 |
| | | S6Δ4 | | | |
| | GM_S22873199 | S6Δ4; | 34 | None | 0 |
| | | S5Δ6 | | | |
| | GM_S22873228 | S6Δ4; | 43 | S5Δ6 (29) | 1 |
| | | S3Δ7 | | | |
| | GM_S22873231 | S8s1; | 40 | S4Δ8 (1), | 8 |
| | | S6Δ4 | | S5Δ5 (1), | |
| | | | | S5Δ6 (2), | |
| | | | | S2Δ4 (1), | |
| | | | | S3Δ6 (1), | |
| | | | | S3Δ8 (1), | |
| | | | | S5Δ14 (1), | |
| | | | | S3Δ9 (1) | |
| | GM_S22873233 | S6Δ4 | 51 | None | 0 |
| GmAP1-like-1- | GM_S22873453 | S6Δ4 | 30 | None | 0 |
| var::LbCas 12a | GM_S22873467 | S6Δ4 | 49 | None | 0 |
| | GM_S22873469 | S1Δ9; | 44 | None | 0 |
| | | S6Δ4 | | | |
| | GM_S22873470 | S-22Δ35; | 51 | None | 0 |
| | | S6Δ4 | | | |
| | GM_S22873477 | S6Δ4 | 75 | S2Δ4 (56), | 3 |
| | | | | Sls1S2Δ5 (1), | |
| | | | | S-2Δ49 (1) | |
| | GM_S22873488 | S6Δ4 | 41 | S3Δ6 (1) | 1 |
| | GM_S22873502 | S6Δ4 | 59 | None | 0 |
| | GM_S22873520 | S3Δ9; | 49 | None | 0 |
| | | S6Δ4 | | | |
| | GM_S22873522 | S6Δ4 | 55 | None | 0 |
| | GM_S22873529 | S6Δ4 | 47 | S4Δ3 (2), | 6 |
| | | | | S3Δ7 (2), | |
| | | | | S-1Δ11 (1), | |
| | | | | S1Δ13 (1), | |
| | | | | S1Δ9 (1), | |
| | | | | S2Δ4 (1) | |
| GmCYC3- | GM_S22873256 | S6Δ4 | 28 | S5Δ6 (1) | 1 |
| 1::LbCas12a | GM_S22873152 | S6Δ4 | 34 | None | 0 |
| | GM_S22873157 | S1Δ13 | 30 | None | 0 |
| | GM_S22873164 | NA | 37 | S6Δ4 (23) | 1 |
| | GM_S22873263 | S6Δ4 | 38 | None | 0 |
| | GM_S22873166 | S6Δ4 | 15 | None | 0 |
| | GM_S22873161 | S6Δ4; S3Δ8 | 30 | S-1Δ10 (1) | 1 |
| | GM_S22873158 | S6Δ4 | 35 | S5Δ6 (1) | 1 |
| | GM_S22873155 | NA | 46 | S6Δ4 (38), | 3 |
| | | | | S3Δ8 (1), | |
| | | | | S5Δ6 (1) | |
| | GM_S22873149 | S6Δ4 | 48 | S3Δ8 (1) | 1 |
| GmAP3-like- | GM_S22873035 | S6Δ4 | 10 | S1Δ13 (1), | 3 |
| 1::LbCas12a | | | | S-3Δ23 (1), | |
| | | | | wt/S3Δ108 (1) | |
| | GM_S22873036 | NA | 7 | S6Δ4 (5) | 1 |
| | GM_S22873037 | S6Δ4 | 11 | S3Δ6 (1), | 2 |
| | | | | S7Δ8 (1) | |
| | | | | S-9Δ16 (1), | |
| | | | | S6Δ96 (1), | |
| | | | | S-2Δ85 (3), | |
| | | | | S-3Δ25 (1), | |
| | GM_S22873043 | NA | 19 | S5Δ6 (1), | 9 |
| | | | | S6Δ4 (4), | |
| | | | | S1Δ9 (1), | |
| | | | | S-4Δ13 (1), | |
| | | | | S-3Δ12 (1) | |
| | GM_S22873050 | NA | 11 | None | 0 |
| | GM_S22873056 | S6Δ4 | 4 | S-2Δ11 (1) | 1 |
| | GM_S22873059 | NA | 8 | S-104Δ117 (1), | 2 |
| | | | | S6Δ4 (8) | |
| | GM_S22873062 | NA | 8 | S3Δ8 (1), | 5 |
| | | | | S-2Δ11 (1), | |
| | | | | S-4Δ10 (1), | |
| | | | | S1Δ58 (1), | |
| | | | | S6Δ4 (4) | |
| | GM_S22873080 | S6Δ4 | 5 | S-1Δ8 (1) | 1 |
| | GM_S22873098 | S-2Δ16; | 6 | S6Δ4 (2) | 1 |
| | | S-3Δ15 | | | |
| GmERL1- | GM_S22873348 | S6Δ4; | 10 | S4Δ3 (1), | 2 |
| var::LbCas12a | | S5Δ7 | | S5Δ6 (1) | |
| | GM_S22873366 | S6Δ4 | 29 | S4Δ3 (10), | 8 |
| | | | | S-3Δ17 (1), | |
| | | | | S-2Δ18 (1), | |
| | | | | S-1Δ30 (1), | |
| | | | | S5Δ5 (1), | |
| | | | | S3Δ9 (1), | |
| | | | | S1Δ9 (1), | |
| | | | | S4Δ8 (1) | |
| | GM_S22873390 | S6Δ4 | 22 | S1Δ29 (1), | 8 |
| | | | | S-1Δ10 (1), | |
| | | | | S3Δ5 (1), | |
| | | | | S3Δ9 (1), | |
| | | | | S5Δ6 (1), | |
| | | | | S7Δ31 (1), | |
| | | | | S-1Δ14 (1), | |
| | | | | S5Δ5 (1) | |
| | GM_S22873403 | S6Δ4 | 4 | None | 0 |
| | GM_S22873392 | S6Δ4 | 21 | S5Δ5 (5) | 1 |
| | GM_S22873384 | S6Δ4; | 27 | S4Δ3 (1), | 4 |
| | | S3Δ8 | | S1Δ14 (1), | |
| | | | | S2Δ4 (1), | |
| | | | | S5Δ5 (2) | |
| | GM_S22873358 | S6Δ4 | 23 | S5Δ5 (2) | 1 |
| | GM_S22873351 | S6Δ4; | 55 | S4Δ3 (13), | 10 |
| | | S-1Δ30; | | S1Δ11 (1), | |
| | | S5Δ5 | | S2Δ4 (1), | |
| | | | | S3Δ8 (3), | |
| | | | | S3Δ7 (2), | |
| | | | | S3Δ6 (1), | |
| | | | | S1Δ9 (2), | |
| | | | | S6s1S7Δ70 (1), | |
| | | | | S1s6S8s3S13s1S17i4 | |
| | | | | (1),* | |
| | | | | S-4Δ13 (1) | |
| | GM_S22873338 | S6Δ4; | 41 | S7Δ8 (1), | 6 |
| | | S4Δ3; | | S3Δ5 (1), | |
| | | S3Δ6; | | S-2Δ61 (1), | |
| | | S5Δ6 | | S3Δ8 (1), | |
| | | | | S4Δ8 (1), | |
| | | | | S6Δ15 (1) | |
| | GM_S22873388 | S6Δ4 | 29 | S4Δ5 (1), | 8 |
| | | | | S3Δ8 (1), | |
| | | | | S5Δ5 (5), | |
| | | | | S1Δ9 (1), | |
| | | | | S5Δ7 (1), | |
| | | | | S-2Δ16 (2), | |
| | | | | S3Δ18 (1), | |
| | | | | S5Δ6 (1) | |

NA—no edits were detected.
*This edit has a 20 bp substitution in place of the first 16 bp of the target site probably arising from multiple repair events

### Example 8. Preferential Expression of Cas12a in Meristematic Tissue Using a Promoter Variant of AtERl1

This example describes the use of a variant of the Arabidopsis thaliana meristematic tissue-preferred promoter AtERl1 to drive the expression of Cas12a expression so as to generate diverse mutations at R0 generation and beyond.

The AtERL1 promoter sequence disclosed as SEQ ID NO:11 comprises a string of 34 Ts starting from nucleotide position 2021 to 2054. Long stretches of a same nucleotide can create issues while sequencing DNA. To overcome this potential problem, a variant of promoter AtERL1 (AtERL1-var) is generated. AtERL1-var (SEQ ID NO: 49) comprises a T to C substitution at positions 2031 and 2043. The substitutions are not predicted to significantly alter the expression activity of the AtERL1 promoter.

An Agrobacterium T-DNA construct 101 is generated. It is similar to Construct 94 except that the LbCas12a cassette is driven by AtERL1-var promoter (SEQ ID NO: 49).

TABLE 15
Table 2. Constructs designed to express a gRNA preferentially,
or solely, in soy or corn floral tissue

| | Promoter::LbCas12a::Mt | | Promoter |
|---|---|---|---|
| Construct | term. | Expression | seq used |
| 94 | AtERL1::LbCas12a | Axillary | 11 |
| | | meristem | |
| 101 | AtERL1-var::LbCas12a | Axillary | 49 |
| | | meristem | |

Soy A3555 cultivar embryos are transformed with the vectors described above by Agrobacterium-mediated transformation and R0 plants are regenerated. DNA is extracted from leaf samples of R0 seedlings for each of Construct 94 and Construct 101. The GmE1 site is sequenced and analyzed for the presence of targeted mutations. Co-expression of Cas12a and its cognate gRNA is expected to generate a double stranded break at the target sites and subsequent imperfect DNA repair generates unique mutations. Several R0 lines from each transformed construct are grown to maturity and at least one ear from each transformed soy plant is self-pollinated. Several hundred R1 lines are selected, germinated and screened for mutations. Edits in R1 progenies derived from Construct 94 and Construct 95 transgenic events are compared.

### Example 9. Use of the Promoter Variant of AtERL1 does not Affect Specificity Nor Strength of Expression of a Reporter Gene

The AtERL1 promoter variant described in Example 8 (SEQ ID NO: 49) was operably linked to the β-glucuronidase reporter gene (GUS) and an Agrobacterium T-DNA construct was generated. As a control, the unmodified AtERL1 promoter (SEQ ID NO: 11) was operably linked to the β-glucuronidase reporter gene (GUS) and used to generate an Agrobacterium T-DNA construct. Soy A3555 cultivar embryos were transformed with the vectors described above by Agrobacterium-mediated transformation and transgenic plants were regenerated.

Quantitative GUS analysis was performed on various vegetative tissue (V5 roots, leaves, petioles) and reproductive tissues (flowers, pollen, immature seeds, pods, seed embryo, seed cotyledons). The spatial expression pattern and strength of expression using the variant AtERL1 promoter was similar to that obtained with the unmodified AtERL1 promoter.

Various tissues from the transgenic soybean plants (different events) were subjected to histochemical expression analysis and expression evidenced by the development of the blue colored spots was mainly restricted to meristematic tissue, also in the soybean plants transformed by the GUS gene operably linked to the variant AtERL1 promoter. Some residual blue staining was observed in restricted flower tissues, obtained from transgenic plants comprising the control construct or the construct with the variant promoter.

The modification in the variant AtERL1 promoter did not affect tissue-specificity nor strength of expression of the promoter.

1. A plant comprising:
(a) a first nucleic acid sequence encoding a guided nuclease capable of generating a staggered cut in a double-stranded DNA molecule operably linked to a heterologous floral tissue-preferred promoter or floral cell-preferred promoter; and
(b) a second nucleic acid sequence encoding at least one guide nucleic acid operably linked to a heterologous second promoter, wherein the at least one guide nucleic acid is capable of hybridizing to a target sequence within a genome of the plant; or
(c) a first nucleic acid sequence encoding a guided nuclease capable of generating a staggered cut in a double-stranded DNA molecule operably linked to a first heterologous promoter; and
(d) a second nucleic acid sequence encoding at least one guide nucleic acid operably linked to a heterologous floral tissue-preferred promoter or floral cell-preferred promoter.

2. The plant of claim 1, wherein the guided nuclease is selected from the group consisting of Cas12a, MAD7® and CasX.

3. The plant of claim 2, wherein the Cas12a is selected from the group consisting of LbCas12a and FnCas12a.

4. The plant of claim 2, wherein the first nucleic acid sequence comprises a nucleic acid sequence at least 90% identical to SEQ ID NO: 32 or SEQ ID NO: 36.

5. The plant of claim 1, wherein the first nucleic acid sequence is codon-optimized for the plant.

6. The plant of claim 1, wherein the first nucleic acid sequence encodes at least one nuclear localization signal.

7. The plant of claim 6, wherein the at least one nuclear localization signal comprises a nucleic acid sequence selected from the group consisting of SEQ ID NOs: 33 and 34.

8. The plant of claim 1, wherein the floral cell-preferred promoter is a floral cell-specific promoter.

9. The plant of claim 1, wherein the floral tissue-preferred promoter is a floral tissue-specific promoter.

10. The plant of claim 1, wherein the floral cell-preferred promoter is selected from the group consisting of an A gene promoter, a B gene promoter, a C gene promoter, a D gene promoter, and an E gene promoter.

11. The plant of claim 1, wherein the floral cell-preferred promoter or floral tissue-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

12. The plant of claim 1, wherein the floral cell-preferred promoter or floral tissue-preferred promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49 or a functional fragment thereof.

13. The plant of claim 1, wherein the first or second promoter is selected from the group consisting of a tissue-preferred promoter, a tissue-specific promoter, an inducible promoter, and a constitutive promoter.

14. The plant of claim 1, wherein the first or second promoter is a floral cell-preferred promoter or a floral tissue-preferred promoter.

15. The plant of claim 1, wherein the first or second promoter is floral cell-specific promoter or a floral tissue-specific promoter.

16. The plant of claim 1, wherein the first or second promoter is selected from the group consisting of an A gene promoter, a B gene promoter, a C gene promoter, a D gene promoter, and an E gene promoter.

17. The plant of claim 1, wherein the first or second promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

18. The plant of claim 1, wherein the first or second promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

19. The plant of claim 13, wherein the constitutive promoter is selected from the group consisting of a CaMV 35S promoter, an Actin promoter, a Rab15 promoter, and a Ubiquitin promoter.

20. The plant of claim 1, wherein the at least one guide nucleic acid comprises at least one guide RNA.

21. The plant of claim 1, wherein the first nucleic acid sequence, the second nucleic acid sequence, or both, are stably integrated into a genome of the plant.

22. The plant of claim 1, wherein the guided nuclease and the at least one guide RNA form a ribonucleoprotein in a floral cell.

23. The plant of claim 22, wherein the ribonucleoprotein generates at least one double-stranded break within the target site in a floral cell.

24. The plant of claim 1, wherein the plant is selected from the group consisting of a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, and a potato plant.

25. The plant of claim 1, wherein the genome is selected from the group consisting of a nuclear genome, a mitochondrial genome, and a plastid genome.

26. A seed produced by the plant of claim 1.

27. The seed of claim 26, wherein the seed comprises at least one mutation in a gene of interest comprising the target sequence as compared to a seed from a control plant of the same variety that lacks the first nucleic acid sequence or second nucleic acid sequence.

28. The seed of claim 26, wherein the at least one mutation in the gene of interest results in the deletion of one or more amino acids from a protein encoded by the gene of interest as compared to a wild-type protein.

29. The seed of claim 26, wherein the at least one mutation in the gene of interest results in the substitution of one or more amino acids within a protein encoded by the gene of interest as compared to a wild-type protein.

30. The seed of claim 26, wherein the at least one mutation in the gene of interest results in the introduction of a premature stop codon in a messenger RNA encoded by the gene of interest as compared to a wild-type messenger RNA.

31. The seed of claim 26, wherein the at least one mutation in the gene of interest comprises the deletion of one or more splice sites from the gene of interest.

32. The seed of claim 26, wherein the seed is a hybrid seed.

33. The seed of claim 26, wherein the seed is an inbred seed.

34. A method of editing a genome of a plant comprising:
(a) introducing into a plant cell:
(i) a first nucleic acid sequence encoding a guided nuclease capable of generating a staggered cut in a double-stranded DNA molecule operably linked to a heterologous floral cell-preferred promoter; and
(ii) a second nucleic acid sequence encoding at least one guide nucleic acid operably linked to a heterologous second promoter, wherein the at least one guide nucleic acid is capable of hybridizing to a target sequence within the genome; and
(b) regenerating at least one plant from the plant cell of step (a), wherein the guided nuclease and at least one guide nucleic acid form a ribonucleoprotein within at least one floral cell of the plant, and wherein the ribonucleoprotein generates at least one double-stranded break within the target sequence in the at least one floral cell.

35. A method of editing a genome of a plant cell comprising:
(a) crossing a first plant with a second plant, wherein the first plant comprises a first nucleic acid sequence encoding a guided nuclease capable of generating a staggered cut in a double-stranded DNA molecule operably linked to a heterologous floral tissue-preferred promoter, and wherein the second plant comprises a second nucleic acid sequence encoding at least one guide nucleic acid operably linked to a heterologous second promoter, wherein the at least one guide nucleic acid is capable of hybridizing to a target sequence within the genome; and
(b) obtaining at least one embryo from the crossing of step (a), wherein the guided nuclease and the at least one guide nucleic acid form a ribonucleoprotein within the at least one embryo, and wherein the ribonucleoprotein generates at least one double-stranded break within the target sequence in the at least one embryo.

36. A method of editing a genome of a plant comprising:
(a) introducing into a plant cell:
(i) a first nucleic acid sequence encoding a guided nuclease capable of generating a staggered cut in a double-stranded DNA molecule operably linked to a heterologous floral tissue-preferred promoter; and
(ii) a second nucleic acid sequence encoding at least one guide nucleic acid operably linked to a heterologous second promoter, wherein the at least one guide nucleic acid is capable of hybridizing to a target sequence within the genome;
(b) regenerating at least one plant from the plant cell of step (a); and
(c) fertilizing the at least one plant to create at least one embryo, wherein the guided nuclease and at least one guide nucleic acid form a ribonucleoprotein within the at least one embryo from step (c), and wherein the ribonucleoprotein generates at least one double-stranded break within the target sequence in the at least one embryo.

37. A method of generating a site-directed integration in a plant comprising:
(a) introducing into a plant cell:
(i) a first nucleic acid sequence encoding a guided nuclease capable of generating a staggered cut in a double-stranded DNA molecule operably linked to a heterologous floral cell-preferred promoter;
(ii) a second nucleic acid sequence encoding one or more guide nucleic acids operably linked to a heterologous second promoter, wherein the one or more guide nucleic acids are
(A) capable of hybridizing to a target sequence within a genome of the plant; and
(B) capable of hybridizing to a first site and a second site flanking a nucleic acid sequence encoding a gene of interest; and
(iii) a third nucleic acid sequence encoding the gene of interest; and
(b) regenerating at least one plant from the plant cell of step (a);
wherein the guided nuclease and at least one guide RNA form a ribonucleoprotein within at least one floral cell of the plant, wherein the ribonucleoprotein generates a double-stranded break within the target sequence molecule, the first site, and the second site, and wherein the gene of interest is integrated into the target sequence in the at least one floral cell.

38. A method of generating a site-directed integration in a plant comprising:
(a) introducing into a plant cell:
(i) a first nucleic acid sequence encoding a guided nuclease capable of generating a staggered cut in a double-stranded DNA molecule operably linked to a heterologous floral tissue-preferred promoter;
(ii) a second nucleic acid sequence encoding one or more guide nucleic acids operably linked to a heterologous second promoter, wherein the one or more guide nucleic acids are
(A) capable of hybridizing to a target sequence within a genome of the plant; and
(B) capable of hybridizing to a first site and a second site flanking a nucleic acid sequence encoding a gene of interest;
(iii) a third nucleic acid sequence encoding the gene of interest;
(b) regenerating at least one plant from the plant cell of step (a); and
(c) fertilizing the at least one plant from step (b) to create at least one embryo;
wherein the guided nuclease and at least one guide RNA form a ribonucleoprotein within at least one embryo, wherein the ribonucleoprotein generates a double-stranded break within the target DNA molecule, the first site, and the second site, and wherein the gene of interest is integrated into the target sequence in the at least one embryo.

39. A method of editing a genome of a plant comprising:
(a) introducing into a plant cell:
(i) a first nucleic acid sequence encoding a guided nuclease capable of generating a staggered cut in a double-stranded DNA molecule operably linked to a heterologous first promoter; and
(ii) a second nucleic acid sequence encoding at least one guide nucleic acid operably linked to a heterologous floral cell-preferred or floral tissue-preferred promoter, wherein the at least one guide nucleic acid is capable of hybridizing to a target sequence within the genome; and
(b) regenerating at least one plant from the plant cell of step (a), wherein the guided nuclease and at least one guide nucleic acid form a ribonucleoprotein within at least one floral cell of the plant, and wherein the ribonucleoprotein generates at least one double-stranded break within the target sequence in the at least one floral cell.

40. A method of editing a genome of a plant cell comprising:
(a) crossing a first plant with a second plant, wherein the first plant comprises a first nucleic acid sequence encoding a guided nuclease capable of generating a staggered cut in a double-stranded DNA molecule operably linked to a heterologous floral tissue-preferred promoter, and wherein the second plant comprises a second nucleic acid sequence encoding at least one guide nucleic acid operably linked to a heterologous second promoter, wherein the at least one guide nucleic acid is capable of hybridizing to a target sequence within the genome; and
(b) obtaining at least one progeny plant from the crossing of step (a), wherein the guided nuclease and the at least one guide nucleic acid form a ribonucleoprotein within at least one floral cell, and wherein the ribonucleoprotein generates at least one double-stranded break within the target sequence in the at least one floral cell.

41. A method of editing a genome of a plant cell comprising:
(a) crossing a first plant with a second plant, wherein the first plant comprises a first nucleic acid sequence encoding a guided nuclease capable of generating a staggered cut in a double-stranded DNA molecule operably linked to a heterologous floral cell-preferred promoter, and wherein the second plant comprises a second nucleic acid sequence encoding at least one guide nucleic acid operably linked to a heterologous second promoter, wherein the at least one guide nucleic acid is capable of hybridizing to a target sequence within the genome; and
(b) obtaining at least one progeny plant from the crossing of step (a), wherein the guided nuclease and the at least one guide nucleic acid form a ribonucleoprotein within at least one floral cell, and wherein the ribonucleoprotein generates at least one double-stranded break within the target sequence in the at least one floral cell.

42. A method of generating a site-directed integration in a plant comprising:
(a) introducing into a plant cell:
(i) a first nucleic acid sequence encoding a guided nuclease capable of generating a staggered cut in a double-stranded DNA molecule operably linked to a heterologous first promoter;
(ii) a second nucleic acid sequence encoding one or more guide nucleic acids operably linked to a heterologous floral cell-preferred or floral-tissue preferred promoter, wherein the one or more guide nucleic acids are
a. capable of hybridizing to a target sequence within a genome of the plant; and
b. capable of hybridizing to a first site and a second site flanking a nucleic acid sequence encoding a gene of interest; and
(iii) a third nucleic acid sequence encoding the gene of interest; and
(b) regenerating at least one plant from the plant cell of step (a); wherein the guided nuclease and at least one guide RNA form a ribonucleoprotein within at least one floral cell of the plant, wherein the ribonucleoprotein generates a double-stranded break within the target sequence molecule, the first site, and the second site, and wherein the gene of interest is integrated into the target sequence in the at least one floral cell.

43. The method of any one of claims 34-42, wherein the target sequence comprises genic DNA.

44. The method of any one of claims 34-42, wherein the target sequence comprises intergenic DNA.

45. The method of any one of claims 34-36 and 39-41, wherein the target sequence is within a gene of interest.

46. The method of claim 45, wherein the gene of interest encodes a protein or a non-protein-coding RNA.

47. The method of claim 46, wherein the non-protein-coding RNA is selected from the group consisting of a microRNA, a small interfering RNA (siRNA), a trans-acting siRNA, or a precursor thereof.

48. The method of any one of claims 34-42, wherein the guided nuclease is selected from the group consisting of Cas12a, MAD7® and CasX.

49. The method of claim 48, wherein the Cas12a is selected from the group consisting of LbCas12a and FnCas12a.

50. The method of claim 34, 37, or 39-42, wherein the floral cell-preferred promoter is a floral cell-specific promoter.

51. The method of any one of claims 35, 36, or 38, wherein the floral tissue-preferred promoter is a floral tissue-specific promoter.

52. The method of any one of claims 34, 37, or 39-42, wherein the floral cell-preferred promoter is selected from the group consisting of an A gene promoter, a B gene promoter, a C gene promoter, a D gene promoter, and an E gene promoter.

53. The method of any one of claims 34, 37, or 39-42, wherein the floral cell-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

54. The method of any one of claims 34, 37, or 39-42, wherein the floral cell-preferred promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

55. The method of any one of claims 35, 36, or 38, wherein the floral tissue-preferred promoter is selected from the group consisting of an A gene promoter, a B gene promoter, a C gene promoter, a D gene promoter, and an E gene promoter.

56. The method of any one of claims 35, 36, or 38, wherein the floral cell-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

57. The method of any one of claims 35, 36, or 38, wherein the floral tissue-preferred promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

58. The method of any one of claims 34-42, wherein the first or second promoter is selected from the group consisting of a tissue-preferred promoter, a tissue-specific promoter, an inducible promoter, and a constitutive promoter.

59. The method of any one of claims 34-42, wherein the first or second promoter is a floral cell-preferred promoter or a floral tissue-preferred promoter.

60. The method of claim 59, wherein the floral cell-preferred promoter or floral tissue-preferred promoter is selected from the group consisting of an A gene promoter, a B gene promoter, a C gene promoter, a D gene promoter, and an E gene promoter.

61. The method of claim 59, wherein the floral cell-preferred promoter or floral tissue-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

62. The method of claim 59, wherein the floral cell-preferred promoter or floral tissue-preferred promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

63. The method of claim 58, wherein the constitutive promoter is selected from the group consisting of a CAMV35S promoter, an Actin promoter, a Rab15 promoter, and a Ubiquitin promoter.

64. The method of any one of claims 34-42, wherein the one or more guide nucleic acids comprises at least one guide RNA.

65. The method of any one of claims 34-42, wherein the first nucleic acid sequence, the second nucleic acid sequence, or both, are stably integrated into a genome of the plant.

66. The method of any one of claims 34-42, wherein the plant is selected from the group consisting of corn, rice, sorghum, wheat, alfalfa, barley, millet, rye, sugarcane, cotton, soybean, canola, tomato, and potato.

67. The method of any one of claims 34-42, wherein the genome is selected from the group consisting of a nuclear genome, a mitochondrial genome, and a plastid genome.

68. The method of any one of claims 34-36 or 39-41, wherein repair of the double-stranded break generates at least one mutation in the target sequence as compared to a control plant of the same line or variety that lacks the first nucleic acid sequence or second nucleic acid sequence.

69. The method of claim 68, wherein the at least one mutation in the target sequence results in the deletion of one or more amino acids from a protein encoded by a gene of interest as compared to a wildtype protein.

70. The method of claim 68, wherein the at least one mutation in the target sequence results in the substitution of one or more amino acids within a protein encoded by a gene of interest as compared to a wildtype protein.

71. The method of claim 68, wherein the at least one mutation in the target sequence results in the introduction of a premature stop codon in a messenger RNA encoded by a gene of interest as compared to a wildtype messenger RNA.

72. The method of claim 68, wherein the at least one mutation in the target sequence comprises the deletion of one or more splice sites from a gene of interest.

73. A recombinant DNA construct comprising (a) a first nucleic acid sequence encoding a guided nuclease capable of generating a staggered cut in a double-stranded DNA molecule operably linked to a heterologous floral cell-preferred or floral tissue-preferred promoter; and (b) a second nucleic acid sequence encoding at least one guide nucleic acid operably linked to a heterologous second promoter, or comprising (c) a first nucleic acid sequence encoding a guided nuclease capable of generating a staggered cut in a double-stranded DNA molecule operably linked to a heterologous promoter; and (d) a second nucleic acid sequence encoding at least one guide nucleic acid operably linked to a heterologous promoter floral cell-preferred or floral tissue-preferred promoter, wherein the at least one guide nucleic acid is capable of hybridizing to a target sequence within a genome of a plant.

74. The recombinant DNA construct of claim 73, wherein the sequence encoding the at least one guide nucleic acid is flanked by self-cleaving ribozymes.

75. A method of generating two or more progeny plants with unique edits from a single transformed plant cell, the method comprising:
(a) introducing into the plant cell:
(i) a first nucleic acid sequence encoding a guided nuclease capable of generating a staggered cut in a double-stranded DNA molecule operably linked to a heterologous floral cell-preferred promoter; and
(ii) a second nucleic acid sequence encoding at least one guide nucleic acid operably linked to a heterologous second promoter, wherein the at least one guide nucleic acid is capable of hybridizing to a target sequence within the genome; and
(b) regenerating a first plant from the plant cell of step (a), wherein the guided nuclease and at least one guide nucleic acid form a ribonucleoprotein within at least one floral cell of the first plant, and wherein the ribonucleoprotein generates at least one double-stranded break within the target sequence in the at least one floral cell;
(c) pollinating the first plant of step (b); and
(d) germinating two or more seeds produced from step (c) to produce two or more progeny plants with unique edits.

76. A method of generating two or more progeny plants with unique edits from a single transformed plant cell, the method comprising:
(a) introducing to the plant cell:
(i) a first nucleic acid sequence encoding a guided nuclease capable of generating a staggered cut in a double-stranded DNA molecule operably linked to a heterologous first promoter; and
(ii) a second nucleic acid sequence encoding at least one guide nucleic acid operably linked to a heterologous floral cell-preferred promoter, wherein the at least one guide nucleic acid is capable of hybridizing to a target sequence within the genome; and
(b) regenerating a first plant from the plant cell of step (a), wherein the guided nuclease and at least one guide nucleic acid form a ribonucleoprotein within at least one floral cell of the first plant, and wherein the ribonucleoprotein generates at least one double-stranded break within the target sequence in the at least one floral cell;
(c) pollinating the first plant of step (b); and
(d) germinating two or more seeds produced from step (c) to produced two or more progeny plants with unique edits.

77. A method of generating two or more progeny plants with unique edits from a single transformed plant cell, the method comprising:
(a) introducing to the plant cell:
(i) a first nucleic acid sequence encoding a guided nuclease capable of generating a staggered cut in a double-stranded DNA molecule operably linked to a heterologous floral tissue-preferred promoter; and
(ii) a second nucleic acid sequence encoding at least one guide nucleic acid operably linked to a heterologous second promoter, wherein the at least one guide nucleic acid is capable of hybridizing to a target sequence within the genome; and
(b) regenerating a first plant from the plant cell of step (a);
(c) pollinating the first plant of step (b), wherein the guided nuclease and at least one guide nucleic acid form a ribonucleoprotein within a floral tissue, and wherein the ribonucleoprotein generates at least one double-stranded break within the target sequence in the floral tissue; and
(d) germinating two or more seeds produced from step (c) to produced two or more progeny plants with unique edits.

78. A method of generating two or more progeny plants with unique edits from a single transformed plant cell, the method comprising:
(a) introducing to the plant cell:
(i) a first nucleic acid sequence encoding a guided nuclease capable of generating a staggered cut in a double-stranded DNA molecule operably linked to a heterologous first promoter; and
(ii) a second nucleic acid sequence encoding at least one guide nucleic acid operably linked to a heterologous floral tissue-preferred promoter, wherein the at least one guide nucleic acid is capable of hybridizing to a target sequence within the genome; and
(b) regenerating a first plant from the plant cell of step (a);
(c) pollinating the first plant of step (b), wherein the guided nuclease and at least one guide nucleic acid form a ribonucleoprotein within a floral tissue, and wherein the ribonucleoprotein generates at least one double-stranded break within the target sequence in the floral tissue; and
(d) germinating two or more seeds produced from step (c) to produced two or more progeny plants with unique edits.

79. The method of any one of claims 75-78, wherein the target sequence comprises genic DNA.

80. The method of any one of claims 75-78, wherein the target sequence comprises intergenic DNA.

81. The method of any one of claims 75-78, wherein the target sequence is within a gene of interest.

82. The method of claim 81, wherein the gene of interest encodes a protein or a non-protein-coding RNA.

83. The method of claim 82, wherein the non-protein-coding RNA is selected from the group consisting of a microRNA, a small interfering RNA (siRNA), a trans-acting siRNA, or a precursor thereof.

84. The method of any one of claims 75-78, wherein the guided nuclease is selected from the group consisting of Cas12a, MAD7® and CasX.

85. The method of claim 84, wherein the Cas12a is selected from the group consisting of LbCas12a and FnCas12a.

86. The method of claim 75 or 76, wherein the floral cell-preferred promoter is a floral cell-specific promoter.

87. The method of any one of claims 77 or 78, wherein the floral tissue-preferred promoter is a floral tissue-specific promoter.

88. The method of claim 75 or 76, wherein the floral cell-preferred promoter is selected from the group consisting of an A gene promoter, a B gene promoter, a C gene promoter, a D gene promoter, and an E gene promoter.

89. The method of claim 75 or 76, wherein the floral cell-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

90. The method of claim 75 or 76, wherein the floral cell-preferred promoter comprises a nucleic acid sequence at least 90% identical to a nucleic acid sequence selected from the group consisting of SEQ ID NOs:1-30, or selected from the group consisting of SEQ ID NOs: 1-16, 18-19, 21-30, 45-49, or a functional fragment thereof.

91. The method of claim 77 or 78, wherein the floral tissue-preferred promoter is selected from the group consisting of an A gene promoter, a B gene promoter, a C gene promoter, a D gene promoter, and an E gene promoter.

92. The method of claim 77 or 78, wherein the floral tissue-preferred promoter is selected from the group consisting of an AP1 promoter, an AP2 promoter, a ZAP1 promoter, an AP3 promoter, a PI promoter, a ZMM16 promoter, a ZMM18 promoter, an AG promoter, a ZAG1 promoter, a ZMM2 promoter, a ZMM23 promoter, an AGL11/STK promoter, an AGL1/SHP1 promoter, an AGL5/SHP2 promoter, a ZAG2 promoter, a ZMM1 promoter, a SEP1 promoter, a SEP2 promoter, a SEP3 promoter, a SEP4 promoter, a ZAG3 promoter, and a ZMM7/SEP-like promoter.

93. The method of claim 77 or 78, wherein the floral tissue-preferred promoter comprises a nucleic acid sequence at least 90% identical to a nucleic acid sequence selected from the group consisting of SEQ ID NOs: 1-30, or selected from the group consisting of SEQ ID NOs: 1-16, 18-19, 21-30, 45-49, or a functional fragment thereof.

94. The method of claim 75 or 77, wherein the second promoter is selected from the group consisting of a tissue-preferred promoter, a tissue-specific promoter, an inducible promoter, and a constitutive promoter.

95. The method of claim 76 or 79, wherein the first promoter is selected from the group consisting of a tissue-preferred promoter, a tissue-specific promoter, an inducible promoter, and a constitutive promoter.

96. The method of claim 94 or 95, wherein the constitutive promoter is selected from the group consisting of a CAMV35S promoter, an Actin promoter, a Rab15 promoter, DAMV promoter and a Ubiquitin promoter.

97. The method of any one of claims 75-78, wherein the one or more guide nucleic acids comprises at least one guide RNA.

98. The method of any one of claims 75-78, wherein the first nucleic acid sequence, the second nucleic acid sequence, or both, are stably integrated into a genome of the first plant.

99. The method of any one of claims 75-78, wherein the plant cell is selected from the group consisting of corn, rice, sorghum, wheat, alfalfa, barley, millet, rye, sugarcane, cotton, soybean, canola, tomato, and potato.

100. The method of any one of claims 75-78, wherein the genome is selected from the group consisting of a nuclear genome, a mitochondrial genome, and a plastid genome.

101. The method of any one of claims 75-78, wherein repair of the double-stranded break generates at least one mutation in the target sequence as compared to a control plant of the same line or variety that lacks the first nucleic acid sequence or second nucleic acid sequence.

102. The method of claim 101, wherein the at least one mutation in the target sequence results in the deletion of one or more amino acids from a protein encoded by a gene of interest as compared to a wildtype protein.

103. The method of claim 101, wherein the at least one mutation in the target sequence results in the substitution of one or more amino acids within a protein encoded by a gene of interest as compared to a wildtype protein.

104. The method of claim 101, wherein the at least one mutation in the target sequence results in the introduction of a premature stop codon in a messenger RNA encoded by a gene of interest as compared to a wildtype messenger RNA.

105. The method of claim 101, wherein the at least one mutation in the target sequence comprises the deletion of one or more splice sites from a gene of interest.

106. The method of claim 76 or 78, wherein the sequence encoding the at least one guide nucleic acid is flanked by self-cleaving ribozymes.

107. The method of any one of claims 75-78, wherein the first plant is self-pollinated.
